SEQUENCE LISTING (32 sequences)

SEQ ID NO: 1 - protein, 6 aa, Artificial Sequence
  Synthetic Construct
SSYYMA

SEQ ID NO: 2 - protein, 5 aa, Artificial Sequence
  Synthetic Construct
DSYMS

SEQ ID NO: 3 - protein, 17 aa, Artificial Sequence
  Synthetic Construct
DMYPDNGDSSYNQKFRE

SEQ ID NO: 4 - protein, 8 aa, Artificial Sequence
  Synthetic Construct
APRWYFSV

SEQ ID NO: 5 - protein, 11 aa, Artificial Sequence
  Synthetic Construct
RASQDISNYLN

SEQ ID NO: 6 - protein, 7 aa, Artificial Sequence
  Synthetic Construct
YTSRLRS

SEQ ID NO: 7 - protein, 9 aa, Artificial Sequence
  Synthetic Construct
QQGHTLPPT

SEQ ID NO: 8 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 9 - protein, 107 aa, Artificial Sequence
  Synthetic Construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 10 - protein, 5 aa, Artificial Sequence
  Synthetic Construct
NYLIE

SEQ ID NO: 11 - protein, 17 aa, Artificial Sequence
  Synthetic Construct
VINPGSGDTYYSEKFKG

SEQ ID NO: 12 - protein, 5 aa, Artificial Sequence
  Synthetic Construct
DRLDY

SEQ ID NO: 13 - protein, 11 aa, Artificial Sequence
  Synthetic Construct
HASQDISSYIV

SEQ ID NO: 14 - protein, 7 aa, Artificial Sequence
  Synthetic Construct
HGTNLED

SEQ ID NO: 15 - protein, 9 aa, Artificial Sequence
  Synthetic Construct
VHYAQFPYT

SEQ ID NO: 16 - protein, 114 aa, Artificial Sequence
  Synthetic Construct
EVQLVQSGAEVKKPGASVKVSCKASGYAFTNYLIEWVRQAPGQGLEWIGVINPGSGDTYYSEKFKGRVTITRDTSTSTAYLELSSLRSEDTAVYYCARDRLDYWGQGTLVTVSS

SEQ ID NO: 17 - protein, 107 aa, Artificial Sequence
  Synthetic Construct
DIQMTQSPSSLSASVGDRVTITCHASQDISSYIVWYQQKPGKAPKLLIYHGTNLEDGVPSRFSGSGSGTDFTLTISSLQPEDFATYYCVHYAQFPYTFGQGTKVEIK

SEQ ID NO: 18 - protein, 215 aa, Artificial Sequence
  Synthetic Construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 214 aa, Artificial Sequence
  Synthetic Construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 20 - protein, 694 aa, Artificial Sequence
  Synthetic Construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 451 aa, Artificial Sequence
  Synthetic Construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 453 aa, Artificial Sequence
  Synthetic Construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNAYTQKSLSLSPGK

SEQ ID NO: 23 - protein, 463 aa, Artificial Sequence
  Synthetic Construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPGK

SEQ ID NO: 24 - protein, 214 aa, Artificial Sequence
  Synthetic Construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 213 aa, Artificial Sequence
  Synthetic Construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 26 - protein, 17 aa, Artificial Sequence
  Synthetic Construct
AIFTGSGAEYKAEWAKG

SEQ ID NO: 27 - protein, 13 aa, Artificial Sequence
  Synthetic Construct
DAGYDYPTHAMHY

SEQ ID NO: 28 - protein, 11 aa, Artificial Sequence
  Synthetic Construct
RASQGISSSLA

SEQ ID NO: 29 - protein, 7 aa, Artificial Sequence
  Synthetic Construct
GASETES

SEQ ID NO: 30 - protein, 12 aa, Artificial Sequence
  Synthetic Construct
QNTKVGSSYGNT

SEQ ID NO: 31 - protein, 123 aa, Artificial Sequence [sequence omitted]

SEQ ID NO: 32 - protein, 110 aa, Artificial Sequence [sequence omitted]

(19) United States
(12) Patent Application Publication
(10) Pub. No.: US 2019/0169667 A1
(43) Pub. Date: Jun. 6, 2019

(54) METHODS OF DECREASING TRISULFIDE BONDS DURING RECOMBINANT PRODUCTION OF POLYPEPTIDES

(71) Applicant: GENENTECH, INC., South San Francisco, CA (US)

(72) Inventors: Martin GAWLITZEK, Redwood City, CA (US); Sven MARKERT, Sindelsdorf (DE); Oliver POPP, Penzberg (DE); Masaru Ken SHIRATORI, Burlingame, CA (US); Thomas TROEBS, Muenchen (DE); Jessica WUU, South San Francisco, CA (US)

(73) Assignee: GENENTECH, INC., South San Francisco, CA (US)

(21) Appl. No.: 16/185,369

(22) Filed: Nov. 9, 2018

Related U.S. Application Data
(63) Continuation of application No. PCT/US2017/031832, filed on May 9, 2017.
(60) Provisional application No. 62/334,433, filed on May 10, 2016.

(51) Int. Cl. C12P 21/00; C12N 5/00; C12N 5/071

(52) U.S. Cl. CPC C12P 21/00; C12N 5/0018; C12N 2500/32; C12N 2500/38; C12N 5/0682

(57) ABSTRACT

Provided herein are cell culture media and methods culturing host cells expressing polypeptides to reduce the level of trisulfide bonds in polypeptides produced by the host cells.

### CROSS REFERENCE TO RELATED APPLICATIONS

This application is a continuation of International Application No. PCT/US2017/031832 filed internationally on May 9, 2017, which claims priority to and the benefit of U.S. Provisional Application Serial No. 62/334,433, filed May 10, 2016, each of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392036501SEQLIST.txt, date recorded: Nov. 7, 2018, size: 35 KB).

### FIELD OF THE INVENTION

The present disclosure relates to cell culture media and methods for decreasing trisulfide bonds in polypeptides produced recombinantly.

### BACKGROUND OF THE INVENTION

Trisulfide bonds are generated by the insertion of an additional sulfur atom into a disulfide bond, thereby resulting in the covalent bonding of three consecutive sulfur atoms. Trisulfide bond formation is a source of heterogeneity in recombinantly produced therapeutic polypeptides. Such heterogeneity is undesirable, as therapeutic products must undergo extensive characterization and meet acceptable standards that ensure product quality and consistency. As such, a demand exists for methods for decreasing the levels of trisulfide bonds during the manufacture of therapeutic polypeptides. There is also a need in the art for minimizing the variability of trisulfide bond levels in therapeutic polypeptides during manufacture. The present disclosure is directed to this and other needs.

### BRIEF SUMMARY OF THE INVENTION

Provided is a method for decreasing trisulfide bond levels in a polypeptide comprising: (a) contacting a host cell comprising a nucleic acid encoding the polypeptide with a basal medium, wherein the basal medium comprises one or more of the following components: i) between about 2 μM to about 35 μM iron, ii) between about 0.11 μM to about 2 μM riboflavin (vitamin B2), iii) between about 4.5 μM to about 80 μM pyridoxine or pyridoxal (vitamin B6),iv) between about 3.4 μM to about 23 μM folic acid (vitamin B9), v) between about 0.2 μM to about 2.5 μM cyanocobalamin (vitamin B12), vi) between about 9 mM and about 10 mM hypotaurine; and vii) between about 0 and about 1.58 mM methionine; (b) culturing the host cell to produce the polypeptide; and (c) harvesting the polypeptide produced by the host cell. In a related aspect, provided is a method for producing a polypeptide, comprising contacting a host cell comprising a nucleic acid encoding the polypeptide with a basal medium, wherein the basal medium comprises one or more of the following components: between about 2 μM to about 35 μM iron, between about 0.11 μM to about 2 μM riboflavin (vitamin B2), between about 4.5 μM to about 80 μM pyridoxine or pyridoxal (vitamin B6), between about 3.4 μM to about 23 μM folic acid (vitamin B9), between about 0.2 μM to about 2.5 μM cyanocobalamin (vitamin B12), between about 9 mM and about 10 mM hypotaurine; and between about 0 and about 1.58 mM methionine; (b) culturing the host cell to produce the polypeptide; and (c)harvesting the polypeptide produced by the host cell.

In certain embodiments according to (or as applied to) any of the embodiments above, the harvested polypeptide has a trisulfide bond level less than a polypeptide produced under identical conditions, except that the concentration of the one or more components differs from the concentration specified in (a). In certain embodiments according to (or as applied to) any of the embodiments above, the basal medium lacks cystine. In certain embodiments according to (or as applied to) any of the embodiments above, the basal medium comprises between about 1.4 mM to 3 mM cysteine or cystine. In certain embodiments according to (or as applied to) any of the embodiments above, the basal medium comprises between about 0 mM to about 1.58 mM methionine and between about 0 mM to about 3 mM cysteine. In certain embodiments according to (or as applied to) any of the embodiments above, the basal medium comprises about 6 mM cysteine.

Also provided is a method for decreasing trisulfide bond levels in a polypeptide comprising: (a) culturing a host cell comprising a nucleic acid encoding the polypeptide in a cell culture medium, wherein the cell culture medium comprises one or more of the following components: i) between about 2 μM to about 35 μM iron, ii) between about 0.11 μM to about 2 μM riboflavin (vitamin B2), iii) between about 4.5 μM to about 80 μM pyridoxine or pyridoxal (vitamin B6), iv) between about 3.4 μM to about 23 μM folate/folic acid (vitamin B9), v) between about 0.2 μM to about 2.5 μM cyanocobalamin (vitamin B12), vi) between about 9 mM and about 10 mM hypotaurine; and vii) between about 0 and about 4.5 mM methionine; (b) producing the polypeptide; (c) and harvesting the polypeptide produced by the host cell. In certain embodiments according to (or as applied to) any of the embodiments above, the concentration of one or more of the components in the cell culture medium is the cumulative concentration of one or more additions after inoculation.

Provided is a method for decreasing trisulfide bond levels in a polypeptide selected from the group consisting of: a CEA-IL2v immunocytokine, a FAP-IL2v immunocytokine, an anti-CEA/anti-CD3 bispecific antibody, an anti-VEGF/anti-angiopoietin bispecific antibody, an anti-Ang2/anti-VEGF bispecific antibody, an anti-C5 antibody, and an anti-CD40 antibody, the method comprising: (a) culturing a host cell comprising a nucleic acid encoding the polypeptide in a cell culture medium, wherein the cell culture medium comprises one or more of the following components: i) between about 2 μM to about 35 μM iron, ii) between about 0.11 μM to about 2 μM riboflavin (vitamin B2), iii) between about 4.5 μM to about 80 μM pyridoxine or pyridoxal (vitamin B6), iv) between about 3.4 μM to about 23 μM folate/folic acid (vitamin B9), v) between about 0.2 μM to about 2.5 μM cyanocobalamin (vitamin B12), vi) between about 9 mM and about 10 mM hypotaurine; and vii) between about 0 and about 4.5 mM methionine; (b) producing the polypeptide; (c) and harvesting the polypeptide produced by the host cell.

Also provided is a method for decreasing trisulfide bond levels in a polypeptide selected from the group consisting of: a CEA-IL2v immunocytokine, a FAP-IL2v immunocytokine, an anti-CEA/anti-CD3 bispecific antibody, an anti-VEGF/anti-angiopoietin bispecific antibody, an anti-Ang2/anti-VEGF bispecific antibody, an anti-C5 antibody and an anti-CD40 antibody, the method comprising: (a) culturing a host cell comprising a nucleic acid encoding the polypeptide in a cell culture medium, wherein the cell culture medium comprises one or more of the following components: i) between about 2 μM to about 35 μM iron, and ii) between about 0 and about 4.5 mM methionine; (b) producing the polypeptide; and (c) harvesting the polypeptide produced by the host cell.

In certain embodiments according to (or as applied to) any of the embodiments above, the method further comprises at least one feed, and wherein the feed medium lacks one or more of the following: iron, riboflavin, pyridoxine, pyridoxal, folic acid, and cyanocobalamin. In certain embodiments according to (or as applied to) any of the embodiments above, the feed is a batch feed. In certain embodiments according to (or as applied to) any of the embodiments above, the batch feed medium lacks cystine. In certain embodiments according to (or as applied to) any of the embodiments above, the batch feed medium lacks cysteine. In certain embodiments according to (or as applied to) any of the embodiments above, the batch feed medium lacks methionine. In certain embodiments according to (or as applied to) any of the embodiments above, the iron is ferric iron (Fe³⁺) or ferrous iron (Fe²⁺).

In certain embodiments according to (or as applied to) any of the embodiments above, the method further comprises: (I) supplementing the culture of said host cell with a chelating agent and a reducing agent prior to harvest; (II) supplementing a pre-harvest cell culture fluid (PHCCF) of said host cell with a chelating agent and a reducing agent; or (III) supplementing a harvested cell culture fluid (HCCF) of said host cell with a chelating agent and a reducing agent following harvest.

Also provided is a method for decreasing level of trisulfide bonds in a polypeptide produced by a host cell comprising: (i) supplementing a culture of said host cell with a reducing agent and a chelating agent prior to harvest; (ii) supplementing a pre-harvest cell culture fluid (PHCCF) of said host cell with a chelating agent and a reducing agent; or (iii) supplementing a harvested cell culture fluid (HCCF) of said host cell with a reducing agent and a chelating agent.

In certain embodiments according to (or as applied to) any of the embodiments above, the culture, the PHCCF, or the HCCF of said host cell is supplemented with the chelating agent prior to being supplemented with the reducing agent. In certain embodiments according to (or as applied to) any of the embodiments above, the PHCCF, or the HCCF of said host cell is supplemented with the chelating agent between about 60 minutes to about 30 minutes prior to being supplemented with the reducing agent. In certain embodiments according to (or as applied to) any of the embodiments above, the chelating agent and the reducing agent are maintained in the culture, the PHCCF, or the HCCF of said host cell for about 30 minutes to about 4 days. In certain embodiments according to (or as applied to) any of the embodiments above, the culture, the PHCCF, or the HCCF of said host cell is maintained at a temperature between about 15° C. and about 37° C. In certain embodiments according to (or as applied to) any of the embodiments above, the culture, the PHCCF, or the HCCF of said host cell is maintained pH between about 6.5 to about 7.5. In certain embodiments according to (or as applied to) any of the embodiments above, the amount of dissolved oxygen (DO) in the culture, the PHCCF, or the HCCF of said host cell is at least about 15%. In certain embodiments according to (or as applied to) any of the embodiments above, the culture, the PHCCF, or the HCCF of said host cell is maintained at a temperature between about 15° C. and about 37° C. and at a pH between about 6.5 to about 7.5, and wherein the amount of dissolved oxygen (DO) in the culture or HCCF of said host cell is at least about 15%.

In certain embodiments according to (or as applied to) any of the embodiments above, the reducing agent is selected from the group consisting of: glutathione (GSH), L-glutathione (L-GSH), cysteine, L-cysteine, tris(2-carboxyethyl)phosphine hydrochloride (TCEP), 2,3-tert-butyl-4-hydroxyanisole, 2,6-di-tert-butyl-4-methylphenol, 3-aminopropane-1-sulfonic acid, adenosylhomocysteine, anserine, B-alanine, B-carotene, butylated hydroxyanisole, butylated hydroxytoluene, carnosine, carvedilol, curcumin, cysteamine, cysteamine hydrochloride, dexamethasone, diallyldisulfide, DL-lanthionine, DL-thiorphan, ethoxyquin, gallic acid, gentisic acid sodium salt hydrate, glutathione disulfide, glutathione reduced ethyl ester, glycine, hydrocortisone, hypotaurine, isethionic acid ammonium salt, L-cysteine-glutathione Disulfide, L-cysteinesulfinic acid monohydrate, lipoic acid, reduced lipoic acid, mercaptopropionyl glycine, methionine, methylenebis(3-thiopropionic acid), oxalic acid, quercitrin hydrate, resveratrol, retinoic acid, S-carboxymethyl-L-cysteine, selenium, selenomethionine, silver diethyldithiocarbamate, taurine, thiolactic acid, tricine, vitamin C, vitamin E, vitamin B1, vitamin B2, vitamin B3, vitamin B4, vitamin B5, vitamin B6, and vitamin B11. In certain embodiments according to (or as applied to) any of the embodiments above, the reducing agent is selected from the group consisting of: cysteine and L-cysteine. In certain embodiments according to (or as applied to) any of the embodiments above, the reducing agent is L-cysteine, and wherein the L-cysteine is added to the culture or HCCF of said host cell to achieve a final concentration between about 3 mM and about 6 mM.

In certain embodiments according to (or as applied to) any of the embodiments above, the chelating agent is selected from the group consisting of: ethylenediaminetetraacetic acid (EDTA), nitrilotriacetic acid (NTA), ethylenediamine-N,N′-disuccinic acid (EDDS), citrate, oxalate, tartrate, ethylene-bis(oxyethylenenitrilo)tetraacetic acid (EGTA), diethylenetriaminepentaacetic acid (DTPA), 5-sulfosalicylic acid, N,N-dimethyldodecylamine N-oxide, dithiooxamide, ethylenediamine, salicylaldoxime, N-(2′-hydroxyethyl)iminodiacetic acid (HIMDA), oxine quinolinol, and sulphoxine. In certain embodiments according to (or as applied to) any of the embodiments above, the chelating agent is selected from the group consisting of: ethylenediaminetetraacetic acid (EDTA), nitrilotriacetic acid (NTA), ethylenediamine-N,N′-disuccinic acid (EDDS), and citrate. In certain embodiments according to (or as applied to) any of the embodiments above, the chelating agent is added to the culture or the HCCF of said host cell to achieve a final concentration of 20 mM.

In certain embodiments according to (or as applied to) any of the embodiments above, the polypeptide is secreted into the cell culture medium. In certain embodiments according to (or as applied to) any of the embodiments above, the method further comprises a step of purifying the harvested polypeptide. In certain embodiments according to (or as applied to) any of the embodiments above, the host cell is a recombinant host cell. In certain embodiments according to (or as applied to) any of the embodiments above, the host cell is a mammalian cell. In certain embodiments according to (or as applied to) any of the embodiments above, the mammalian cell is a CHO cell. In certain embodiments according to (or as applied to) any of the embodiments above, the method further comprises measuring the level of trisulfide bonds in the polypeptide. In certain embodiments according to (or as applied to) any of the embodiments above, the average % trisulfide bonds in the polypeptide is less than about 20%, less than about 10% less than about 5%, less than about 1%, less than about 0.5%, or less than about 0.1%.

In certain embodiments according to (or as applied to) any of the embodiments above, the polypeptide is an antibody or fragment thereof. In certain embodiments according to (or as applied to) any of the embodiments above, the polypeptide is an antibody fragment, and wherein the antibody fragment is selected from the group consisting of: a Fab, a Fab′, an F(ab′)₂, an scFv, an (scFv)₂, a dAb, a complementarity determining region (CDR) fragment, a linear antibody, a single-chain antibody molecule, a minibody, a diabody, and multispecific antibody formed from antibody fragments. In certain embodiments according to (or as applied to) any of the embodiments above, the antibody or fragment thereof binds to an antigen selected from the group consisting of: BMPR1B, E16, STEAP1, 0772P, MPF, Napi3b, Sema 5b, PSCA hlg, ETBR, MSG783, STEAP2, TrpM4, C5, CRIPTO, CD21, CD79b, FcRH2, HER2, NCA, MDP, IL20Rα, Brevican, EphB2R, ASLG659, PSCA, GEDA, BAFF-R, CD22, CD79a, CXCRS, HLA-DOB, P2X5, CD72, LY64, FcRH1, IRTA2, TENB2, PMEL17, TMEFF1, GDNF-Ra1, Ly6E, TMEM46, Ly6G6D, LGRS, RET, LY6K, GPR19, GPR54, ASPHD1, Tyrosinase, TMEM118, GPR172A, CD33, CLL-1, OX40, α4β7 and αEβ7 integrin heterodimers, IL-13, CD-20, FGFR, influenza A, influenza B, amyloid beta, HER3, complement factor D, IL-22c, PD-L1, PD-L2, PD-1, VEGF, Angiopoietin 2, CD3, FAP, CEA, and IL-6. In certain embodiments according to (or as applied to) any of the embodiments above, the polypeptide is an antibody, and wherein the antibody is a bispecific antibody. In certain embodiments according to (or as applied to) any of the embodiments above, the bispecific antibody is an anti-VEGF/anti-angiopoietin bispecific antibody, an anti-CEA/anti-CD3 bispecific antibody, or an anti-Ang2/anti-VEGF bispecific antibody. In certain embodiments according to (or as applied to) any of the embodiments above, the polypeptide is an immunocytokine. In certain embodiments according to (or as applied to) any of the embodiments above, the immunocytokine is CEA-IL2v or FAP-IL2v.

Also provided is the use of between about 0 and about 4.5 μM methionine in a cell culture medium for decreasing trisulfide bond levels in a polypeptide selected from the group consisting of: a CEA-IL2v immuocytokine, a FAP-IL2v immunocytokine, an anti-CEA/anti-CD3 bispecific antibody, an anti-VEGF/anti-angiopoietin bispecific antibody, an anti-Ang2/anti-VEGF bispecific antibody, an anti-C5 antibody, and an anti-CD40 antibody.

It is to be understood that one, some, or all of the properties of the various embodiments described herein may be combined to form other embodiments of the present invention. These and other aspects of the invention will become apparent to one of skill in the art.

All publications, patents, and patent applications cited herein are incorporated by reference in their entirety for all purposes.

### BRIEF DESCRIPTION OF THE DRAWINGS

FIG. 1 provides the results of experiments performed to assess the % trisulfide bond in anti-FluB incubated in a cell-free system containing Medium 1+cysteine (Cys); Medium 1+Cys+Fe (iron); Medium 1+cystine (Cys-Cys); Medium 1+Cys-Cys+Fe; Medium 2+Cys; Medium 2+Cys+Fe; Medium 2+Cys-Cys; or Medium 1+Cys-Cys+Fe.

FIG. 2A provides the results of experiments performed to assess the % trisulfide bond in anti-FluB incubated in Medium 1 that was supplemented with one or more of the following components: (a) Cys-Cys, (b) Fe, and (c) B vitamins (riboflavin, pyridoxine, folic acid, and cyanocobalamin).

FIG. 2B provides the results of experiments performed to assess the % trisulfide bond in an anti-OX40 antibody incubated in Medium 1 that was supplemented with one or more of the following components: (a) Cys-Cys, (b) Fe, and (c) B vitamins (riboflavin, pyridoxine, folic acid, and cyanocobalamin).

FIG. 3 provides the results of experiments performed to assess the effects of added Cys or added Cys-Cys on trisulfide bond levels in an anti-OX40 antibody produced by a CHO cell culture.

FIG. 4A provides the results of experiments performed to assess the effects of different concentrations of Cys in the basal medium on trisulfide bond levels in an anti-OX40 antibody produced by a CHO cell culture.

FIG. 4B provides the yield of anti-OX40 antibody produced by each cell culture run depicted in FIG. 4A.

FIG. 5A provides the results of experiments performed to assess the effects of providing different concentrations of Cys and Fe in the basal medium as well as different concentrations of B vitamins in the batch feed medium on trisulfide bond levels in an anti-OX40 antibody produced by a CHO cell culture.

FIG. 5B provides the yield of anti-OX40 antibody produced by each cell culture run depicted in FIG. 5A.

FIG. 5C shows residual concentrations of cystine (Cys-Cys) in the medium at the end of each cell culture run in FIG. 5A.

FIG. 6 provides the results of experiments performed to assess the effects of different concentrations Fe in the basal medium and B vitamins in the batch feed medium on trisulfide bond levels in an anti-OX40 antibody produced by a CHO cell culture.

FIG. 7A provides the results of experiments performed to assess the effects of adding cysteine or cysteine+EDTA to the harvested cell culture fluid of an anti-OX40 antibody on trisulfide bond levels in the antibody.

FIG. 7B shows the CE-SDS results of the experiments described in FIG. 7A assessing the amount of disulfide bond reduction in the anti-OX40 antibody maintained under each of the conditions tested in FIG. 7A.

FIG. 8A provides the results of experiments performed to assess the effects of adding cysteine, cysteine+EDTA, cysteine+NTA, cysteine+EDDS, or cysteine+citrate to the cell culture fluid of an anti-OX40 antibody on trisulfide bond levels in the antibody from 0 to 5 hours following addition.

FIG. 8B provides the results of experiments performed to assess the effects of adding cysteine, cysteine+EDTA, cysteine+NTA, cysteine+EDDS, or cysteine+citrate to the cell culture fluid of an anti-OX40 antibody on trisulfide bond levels in the antibody from 0 to 96 hours following addition.

FIG. 9 provides the results of experiments that were conducted to assess the effects of hypotaurine on trisulfide bond formation in an anti-OX40 antibody during cell culture.

FIG. 10 provides a prediction profiler showing a significant impact of lowering methionine concentration on trisulfide bond reduction

FIG. 11 provides the results of experiments that were conducted to assess the effects of providing different concentrations of cysteine and methionine in the basal medium on trisulfide bond levels in a bispecific antibody produced by a CHO cell culture.

FIG. 12 provides the results of experiments that were conducted to assess the effect of omitting B vitamins from the batch feed medium on trisulfide levels in an antibody produced by a CHO cell culture. Two separate runs were performed.

### DETAILED DESCRIPTION

The present invention pertains to methods of preventing, eliminating and/or decreasing the level of trisulfide bonds in polypeptides (such as antibodies and bispecific antibodies) produced in cell culture. In certain aspects, the methods provided herein comprise contacting a host cell comprising a nucleic acid encoding the polypeptide with a basal medium, wherein the basal medium comprises one or more of the following components: i) between about 2 μM to about 35 μM iron, ii) between about 0.11 μM to about 2 μM riboflavin (vitamin B2), iii) between about 4.5 μM to about 80 μM pyridoxine or pyridoxal (vitamin B6), iv) between about 3.4 μM to about 23 μM folate/folic acid (vitamin B9), v) between about 0.2 μM to about 2.5 μM cyanocobalamin (vitamin B12), vi) between about 9 mM and about 10 mM hypotaurine; and vii) between about 0 and about 1.58 mM methionine; culturing the host cell to produce the polypeptide; and harvesting the polypeptide produced by the host cell, whereby a level of trisulfide bonds in the polypeptide is decreased.

In a related aspect, provided are methods for decreasing trisulfide bond levels in a polypeptide selected from the group consisting of: a CEA-IL2v immunocytokine, a FAP-IL2v immunocytokine, an anti-CEA/anti-CD3 bispecific antibody, an anti-VEGF/anti-angiopoietin bispecific antibody, an anti-Ang2/anti-VEGF bispecific antibody, an anti-C5 antibody and an anti-CD40 antibody, which methods comprise: culturing a host cell comprising a nucleic acid encoding the polypeptide in a cell culture medium, wherein the cell culture medium comprises one or more of the following: i) between about 2 μM to about 35 μM iron, ii) between about 0.11 μM to about 2 μM riboflavin (vitamin B2), iii) between about 4.5 μM to about 80 μM pyridoxine or pyridoxal (vitamin B6), iv) between about 3.4 μM to about 23 μM folate/folic acid (vitamin B9), v) between about 0.2 μM to about 2.5 μM cyanocobalamin (vitamin B12), vi) between about 9 mM and about 10 mM hypotaurine; and vii) between about 0 and about 1.58 mM methionine; producing the polypeptide; and harvesting the polypeptide produced by the host cell, whereby a level of trisulfide bonds in the polypeptide is decreased.

Additionally or alternatively, the methods comprise supplementing the culture or cell culture fluid of said host cell, the pre-harvest cell culture fluid (PHCCF) of said host cell, or a harvested cell culture fluid (HCCF) of said host cell with a chelating agent and a reducing agent.

The following description sets forth exemplary methods, parameters, and the like. It should be recognized, however, that such description is not intended as a limitation on the scope of the present disclosure but is instead provided as a description of exemplary embodiments.

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.); Antibodies, A Laboratory Manual, and Animal Cell Culture (R. I. Freshney, ed. (1987)); Methods in Molecular Biology, Humana Press; Cell Biology: Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); and The Antibodies (M. Zanetti and J. D. Capra, eds., Harwood Academic Publishers, 1995).

### A. Definitions

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of aspects and embodiments.

The terms “medium” and “cell culture medium” refer to a nutrient source used for growing or maintaining cells. As is understood by a person of skill in the art, the nutrient source may contain components required by the cell for growth and/or survival and/or product generation, or may contain components that aid in cell growth and/or survival and/or product generation. Vitamins, essential or non-essential amino acids, trace elements, and surfactants (e.g., poloxamers) are examples of medium components.

For example, a “chemically defined cell culture medium” or “CDM” refers to a medium with a specified composition that is free of animal-derived products such as animal serum and peptone. The term also encompass a medium with a specified composition that is free of undefined or partially defined components, for example, components such as animal serum, an animal peptone (hydrolysate), a plant peptone (hydrolysate), and a yeast peptone (hydrolysate). As would be understood by a person of skill in the art, a CDM may be used in a process of polypeptide production whereby a cell is in contact with, and secretes a polypeptide into, the CDM. Thus, it is understood that a composition may contain a CDM and a polypeptide product and that the presence of the polypeptide product does not render the CDM chemically undefined.

A “chemically undefined cell culture medium” refers to a medium whose chemical composition cannot be specified and which may contain one or more animal-derived products such as serum and peptone. As would be understood by a person of skill in the art, a chemically undefined cell culture medium may contain an animal-derived product as a nutrient source. The term can also encompass a cell culture medium comprising undefined or partially defined components, for example, components such as an animal serum, an animal peptone (hydrolysate), a plant peptone (hydrolysate), or a yeast peptone (hydrolysate).

As used herein, “basal medium” refers to cell culture medium containing cell culture nutrients supplied to a culturing vessel at the start of a culturing process. The basal medium can be the medium that cells are inoculated into before a cell culture cycle. The basal cell culture medium can be supplied prior to a cell culture cycle for a batch or fed-batch cell culture. A basal cell culture medium may also be supplied as a feed medium, continuously or in discreet increments, to the cell culture during the culturing process, with or without period cell and/or product harvest before termination of the culture (i.e., fed-batch cell culture).

As used herein, “feed medium” refers to cell culture medium containing cell culture nutrients supplied to a culturing vessel as a feed medium, continuously or in discreet increments, to the cell culture during the culturing process, with or without period cell and/or product harvest before termination of the culture (i.e., fed-batch cell culture).

“Culturing” a cell refers to contacting a cell with a cell culture medium under conditions suitable to the viability and/or growth and/or proliferation of the cell.

“Batch culture” refers to a culture in which all components for cell culturing (including the cells and all culture nutrients and components) are supplied to the culturing vessel at the start of the culturing process.

“Perfusion culture” is a culture by which the cells are restrained in the culture by, e.g., filtration, encapsulation, anchoring to microcarriers, etc., and the culture medium is continuously or intermittently introduced and removed from the culturing vessel.

The phrase “fed batch cell culture,” as used herein refers to a batch culture wherein the cells and culture medium are supplied to the culturing vessel initially, and additional culture nutrients are fed, continuously or in discrete increments, to the culture during the culturing process, with or without periodic cell and/or product harvest before termination of culture.

“Culturing vessel” refers to a container used for culturing a cell. The culturing vessel can be of any size so long as it is useful for the culturing of cells.

The term “trace metals” refers to metals needed by cells in small amounts for growth, survival, and/or product generation. Examples of trace metals encompassed within the definition herein include but are not limited to iron (including ferrous iron (also referred to as Fe (II) or Fe²⁺) and ferric iron (also referred to as Fe (III) or Fe³⁺), magnesium, lithium, silicon, zinc, copper, chromium, nickel, cobalt, manganese, aluminum, vanadium, selenium, tin, cadmium, molybdenum, and titanium.

The term “antioxidant” refers to a molecule that slows the rate of oxidation of other molecules. Examples of antioxidants encompassed within the definition herein include but are not limited to 2,3-tert-butyl-4-hydroxyanisole, 2,6-di-tert-butyl-4-methylphenol, 3-aminopropane-1-sulfonic acid, adenosylhomocysteine, Anserine, B-Alanine, B-carotene, Butylated hydroxyanisole, Butylated hydroxytoluene, Carnosine, Carvedilol, Curcumin, Cysteamine, Cysteamine hydrochloride, Cysteine, Dexamethasone, Diallyldisulfide, DL-Lanthionine, DL-Thiorphan, Ethoxyquin, Gallic acid, Gentisic acid sodium salt hydrate, Glutathione (GSH), Glutathione disulfide, Glutathione reduced ethyl ester, Glycine, Hydrocortisone, Hypotaurine, Isethionic acid ammonium salt, L-Cysteine-glutathione Disulfide, L-Cysteinesulfinic acid monohydrate, Lipoic Acid, Lipoic acid reduced, Mercaptopropionyl glycine, Methionine, Methylenebis(3-thiopropionic acid), Oxalic acid, Quercetrin hydrate, Resveratrol, Retinoic acid, S-Carboxymethyl-L-cysteine, Selenium, Selenomethionine, Silver diethyldithiocarbamate, Taurine, Thiolactic acid, Tricine, Vitamin C, Vitamin E, Vitamin B1, Vitamin B2, Vitamin B3, Vitamin B4, Vitamin B5, Vitamin B6, and Vitamin B11.

A “nucleic acid” refers to polymers of nucleotides of any length, and includes DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer.

An “isolated nucleic acid” means and encompasses a non-naturally occurring, recombinant or a naturally occurring sequence outside of or separated from its usual context. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the protein where, for example, the nucleic acid molecule is in a chromosomal location different from that of natural cells.

An “isolated” protein (e.g., an isolated antibody) is one which has been identified and separated and/or recovered from a component of its natural environment. Contaminant components of its natural environment are materials which would interfere with research, diagnostic or therapeutic uses for the protein, and may include enzymes, hormones, and other proteinaceous or nonproteinaceous solutes. Isolated protein includes the protein in situ within recombinant cells since at least one component of the protein's natural environment will not be present. Ordinarily, however, isolated protein will be prepared by at least one purification step.

A “purified” protein (e.g., antibody) means that the protein has been increased in purity, such that it exists in a form that is more pure than it exists in its natural environment and/or when initially produced and/or synthesized and/or amplified under laboratory conditions. Purity is a relative term and does not necessarily mean absolute purity.

“Contaminants” refer to materials that are different from the desired protein product (e.g., different from an antibody product). A contaminant may include, without limitation: host cell materials, such as CHOP; nucleic acid; a variant, fragment, aggregate or derivative of the desired protein; another polypeptide; endotoxin; viral contaminant; cell culture media components, etc.

The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Examples of polypeptides encompassed within the definition herein include mammalian proteins, such as, e.g., renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4,-5, or -6 (NT-3, NT-4, NT-5, or NT-6), or a nerve growth factor such as NGF-b; platelet-derived growth factor (PDGF); fibroblast growth factor such as αFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins (IGFBPs); CD proteins such as CD3, CD4, CD8, CD19 and CD20; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as 0772P (CA125, MUC16) (i.e., ovarian cancer antigen) or HER2, HER3 or HER4 receptor; immunoadhesins; and fragments and/or variants of any of the above-listed proteins as well as antibodies, including antibody fragments, binding to a protein, including, for example, any of the above-listed proteins.

The term “titer” as used herein refers to the total amount of an expressed protein product produced by a cell culture divided by a given amount of medium volume. Titer can be expressed or assessed in terms of a relative measurement, such as a percentage increase in titer as compared obtaining the protein product under different culture conditions.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. An antibody can be human, humanized and/or affinity matured.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody, preferably comprising the antigen binding region (the term “antigen-binding fragment” may be used interchangeably) thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, in The Pharmacology of Monoclonal Antibodies, vol. 113, Rosenburg and Moore eds., Springer-Verlag, New York, pp. 269-315, 1994.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boemer et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U .S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat Numbering) |
| H1 | H31-H35 | H26-H35 | H26-H32 | H30-H35 |
| | | | | (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile.

“Pharmaceutically acceptable” carriers, excipients, or stabilizers are ones which are nontoxic to the cell or mammal being exposed thereto at the dosages and concentrations employed (Remington's Pharmaceutical Sciences (20^{th} edition), ed. A. Gennaro, 2000, Lippincott, Williams & Wilkins, Philadelphia, Pa.). Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone, amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterfoils such as sodium; and/or nonionic surfactants such as Tween™, polyethylene glycol (PEG), and Pluronics™.

A “sterile” formulation is aseptic or free or essentially free from all living microorganisms and their spores.

The term “pre-harvest cell culture fluid” refers to the fluid present at the end of cell culture, after cell culture, or just before cell harvest. A pre-harvest cell culture fluid includes, but is not limited to, cell culture medium to which one or more agents of the invention are optionally added. A pre-harvest cell culture fluid includes, but is not limited to, cell culture medium from which cells, the contents of cells, and/or cellular debris has not been removed. The cell culture media and/or pre-harvest cell culture fluid may contain proteins or antibodies that are released (e.g., secreted) into the media or solution by the cells during culturing. The conditions for cell culture fluid are optimized for cell growth, whereas the pre-harvest and harvest cell culture fluids may be pre-treated to optimize for cell separation and purification of a polypeptide (such as a recombinant polypeptide, e.g., an antibody) secreted by the host cell. For example, the pre-harvest step may include preparation of the culture for harvest by reducing temperature, changing the pH (usually lowering to a pH of about 5 or to a pH of less than about 7), and flocculation. The pre-harvest step can be optional as the cell culture fluid can be pumped directly from the bioreactor where the cells are being cultured to the centrifuge or filter for the harvesting step. In cases where no pre-treatment is applied prior to harvest, pre-harvest cell culture fluid and cell culture fluid are indistinguishable.

“Harvested cell culture fluid” refers to the fluid present during the cell separation process and after separation of the cells from the cell culture media via methods, such as centrifugation or filtration. A harvested cell culture fluid typically includes polypeptide (such as recombinant polypeptides, e.g., antibodies) secreted by the cells during cell culture.

### B. Cell Culture and Methods of the Invention

### Methods of Decreasing the Level of Trisulfide Bonds in a Polypeptide

Trisulfide bonds are generated by the insertion of an additional sulfur atom into a disulfide bond, thereby resulting in the covalent bonding of three consecutive sulfur atoms. Trisulfide bonds can form between cysteine residues in polypeptides and can form intramolecularly (i.e., between two cysteines in the same polypeptide) or intermolecularly (i.e. between two cysteines in separate polypeptides). Provided herein are methods for decreasing the level of trisulfide bonds in polypeptides during cell culture. Also provided herein are methods for decreasing the level of trisulfide bonds in polypeptides during processing following cell culture. The methods herein can advantageously be used for large scale production of disulfide bond containing polypeptides (e.g., antibodies), such as at a manufacturing scale.

In certain embodiments, a host cell is combined (contacted) with any of the cell culture media (such as basal cell culture media) described herein under conditions that promote, e.g., cell growth, and/or polypeptide production. In certain embodiments, the term “inoculum” refers to a volume of host cells from a seed train for addition to a basal medium. In certain embodiments, the inoculum comprises additional components, e.g., seed train medium. In certain embodiments, the term “initial cell culture medium” refers to the cell culture medium after the inoculum and the basal medium are mixed. In certain embodiments, the inoculum and the basal medium are mixed at a ratio of about any one of 1:5, 1:4.5, 1:4, 1:3.5, or 1:3, including any ratio in between. In certain embodiments, additional components are provided to the culture, either continuously or at one or more discrete intervals, at some time subsequent to the inoculum and basal medium are mixed. In certain embodiments, the term “cumulative” refers to the total amount of a particular component or components added during cell culture, including components added at the beginning of the cell culture and subsequently added components, without considering consumption or generation by the cells.

In certain embodiments, provided is a method for decreasing trisulfide bond levels in a polypeptide comprising: contacting a host cell comprising a nucleic acid encoding the polypeptide with a basal medium, wherein the basal medium comprises one or more of the following components:
i) between about 2 μM to about 35 μM iron,
ii) between about 0.11 μM to about 2 μM riboflavin (vitamin B2),
iii) between about 4.5 μM to about 80 μM pyridoxine or pyridoxal (vitamin B6),
iv) between about 3.4 μM to about 23 μM folate/folic acid (vitamin B9),
v) between about 0.2 μM to about 2.5 μM cyanocobalamin (vitamin B12),
vi) between about 9 mM and about 10 mM hypotaurine; and
vii) between about 0 and about 1.58 mM methionine;
   culturing the host cell to produce the polypeptide; and harvesting the polypeptide produced by the host cell, whereby a level of trisulfide bonds in the polypeptide is decreased. In certain embodiments, the harvested polypeptide has a trisulfide bond level that is less than the trisulfide bond level of a polypeptide produced under identical conditions, except that the concentration of one or more components differs from the concentration(s) specified above. In certain embodiments, the average % trisulfide bond in the harvested polypeptide is less than about any one of 20%, 19%, 18%, 17%, 16%, 15%, 14%, 13%, 12%, 10%, 9%, 8%, 7%, 6%, 5%, 4%, 3%, 2%, 1%, 0.5%, or 0.1% (mol trisulfide/mol polypeptide). In certain embodiments, the average trisulfide in the harvested polypeptide is reduced by about any one of 10%, 20%, 25%, 30%, 35%, 40%, 45%, 50%, 55%, 65%, 70%, 75%, 80%, 85%, 90%, 91%, 92%, 93%, 94%, 95%, 96%, 97%, 98%, 99%, or more than 99% relative to the trisulfide bond level of a polypeptide produced under identical conditions, except that the concentration of one or more components differs from the concentration(s) specified above.

In certain embodiments, provided is a method for producing a polypeptide, comprising: contacting a host cell comprising a nucleic acid encoding the polypeptide with a basal medium, wherein the basal medium comprises one or more of the following components:
i) between about 2 μM to about 35 μM iron,
ii) between about 0.11 μM to about 2 μM riboflavin (vitamin B2),
iii) between about 4.5 μM to about 80 μM pyridoxine or pyridoxal (vitamin B6),
iv) between about 3.4 μM to about 23 μM folate/folic acid (vitamin B9),
v) between about 0.2 μM to about 2.5 μM cyanocobalamin (vitamin B12),
vi) between about 9 mM and about 10 mM hypotaurine; and
vii) between about 0 and about 1.58 mM methionine;
   culturing the host cell to produce the polypeptide; and harvesting the polypeptide produced by the host cell, whereby a level of trisulfide bonds in the polypeptide is decreased. In certain embodiments, the harvested polypeptide has a trisulfide bond level that is less than the trisulfide bond level of a polypeptide produced under identical conditions, except that the concentration of one or more components differs from the concentration(s) specified above. In certain embodiments, the average % trisulfide in the harvested polypeptide is less than about any one of 20%, 19%, 18%, 17%, 16%, 15%, 14%, 13%, 12%, 10%, 9%, 8%, 7%, 6%, 5%, 4%, 3%, 2%, 1%, 0.5%, or 0.1% (mol trisulfide/mol polypeptide). In certain embodiments, the average trisulfide in the harvested polypeptide is reduced by about any one of 10%, 20%, 25%, 30%, 35%, 40%, 45%, 50%, 55%, 65%, 70%, 75%, 80%, 85%, 90%, 91%, 92%, 93%, 94%, 95%, 96%, 97%, 98%, 99%, or more than 99% relative to the trisulfide bond level of a polypeptide produced under identical conditions, except that the concentration of one or more components differs from the concentration(s) specified above.

In certain embodiments, provided is a method for decreasing trisulfide bond levels in a polypeptide comprising: culturing a host cell comprising a nucleic acid encoding the polypeptide in a cell culture medium, wherein the cell culture medium comprises one or more of the following components:

i) between about 2 μM to about 35 μM iron,

ii) between about 0.11 μM to about 2 μM riboflavin (vitamin B2),

iii) between about 4.5 μM to about 80 μM pyridoxine or pyridoxal (vitamin B6),

iv) between about 3.4 μM to about 23 μM folate/folic acid (vitamin B9),

v) between about 0.2 μM to about 2.5 μM cyanocobalamin (vitamin B12),

vi) between about 9 mM and about 10 mM hypotaurine; and

vii) between about 0 and about 4.5 mM methionine;

producing the polypeptide; and harvesting the polypeptide produced by the host cell. In certain embodiments, the concentration of one or more of the components in the cell culture medium is the cumulative concentration of one or more additions after inoculation.

In certain embodiments, provided is a method for decreasing trisulfide bond levels in a polypeptide selected from the group consisting of: a CEA-IL2v immunocytokine, a FAP-IL2v immunocytokine, an anti-CEA/anti-CD3 bispecific antibody, an anti-VEGF/anti-angiopoietin bispecific antibody, an anti-Ang2/anti-VEGF bispecific antibody, an anti-C5 antibody, and an anti-CD40 antibody , the method comprising: culturing a host cell comprising a nucleic acid encoding the polypeptide in a cell culture medium, wherein the cell culture medium comprises one or more of the following components:
i) between about 2 μM to about 35 μM iron,
ii) between about 0.11 μM to about 2 μM riboflavin (vitamin B2),
iii) between about 4.5 μM to about 80 μM pyridoxine or pyridoxal (vitamin B6),
iv) between about 3.4 μM to about 23 μM folate/folic acid (vitamin B9),
v) between about 0.2 μM to about 2.5 μM cyanocobalamin (vitamin B12),
vi) between about 9 mM and about 10 mM hypotaurine; and
vii) between about 0 and about 4.5 mM methionine;
   producing the polypeptide; and harvesting the polypeptide produced by the host cell, whereby a level of trisulfide bonds in the polypeptide is decreased. In certain embodiments, the CEA-IL2v immuocytokine is RG7813. In certain embodiments, the FAP-IL2v immunocytokine is RG7461. In certain embodiments, the anti-CEA/anti-CD3 bispecific antibody is RG7802. In certain embodiments, the anti-VEGF/anti-angiopoietin bispecific antibody is RG7716. In certain embodiments, the anti-Ang2/anti-VEGF bispecific antibody is RG7221. In certain embodiments, the anti-Ang2/anti-VEGF bispecific antibody is CAS Number 1448221-05-3. In certain embodiments, the anti-CD40 antibody is RG7876. In certain embodiments, the cell culture medium is an initial cell culture medium. In certain embodiments, the harvested polypeptide has a trisulfide bond level that is less than the trisulfide bond level of a polypeptide produced under identical conditions, except that the concentration of one or more components differs from the concentration(s) specified above. In certain embodiments, the average % trisulfide in the harvested polypeptide is less than about any one of 20%, 19%, 18%, 17%, 16%, 15%, 14%, 13%, 12%, 10%, 9%, 8%, 7%, 6%, 5%, 4%, 3%, 2%, 1%, 0.5%, or 0.1% (mol trisulfide/mol polypeptide). In certain embodiments, the average trisulfide in the harvested polypeptide is reduced by about any one of 10%, 20%, 25%, 30%, 35%, 40%, 45%, 50%, 55%, 65%, 70%, 75%, 80%, 85%, 90%, 91%, 92%, 93%, 94%, 95%, 96%, 97%, 98%, 99%, or more than 99% relative to the trisulfide bond level of a polypeptide produced under identical conditions, except that the concentration of one or more components differs from the concentration(s) specified above.

In certain embodiments, provided is a method for decreasing trisulfide bond levels in a polypeptide selected from the group consisting of: a CEA-IL2v immuocytokine, a FAP-IL2v immunocytokine, an anti-CEA/anti-CD3 bispecific antibody, an anti-VEGF/anti-angiopoietin bispecific antibody, an anti-Ang2/anti-VEGF bispecific antibody, an anti-C5 antibody, and an anti-CD40 antibody , the method comprising: culturing a host cell comprising a nucleic acid encoding the polypeptide in a cell culture medium, wherein the cell culture medium comprises one or more of the following components:
i) between about 2 μM to about 35 μM iron, and
ii) between about 0 and about 4.5 mM methionine;
   producing the polypeptide; and harvesting the polypeptide produced by the host cell, whereby a level of trisulfide bonds in the polypeptide is decreased. In certain embodiments, the CEA-IL2v immuocytokine is RG7813. In certain embodiments, the FAP-IL2v immunocytokine is RG7461. In certain embodiments, the anti-CEA/anti-CD3 bispecific antibody is RG7802. In certain embodiments, the anti-VEGF/anti-angiopoietin bispecific antibody is RG7716. In certain embodiments, the anti-Ang2/anti-VEGF bispecific antibody is RG7221. In certain embodiments, the anti-Ang2/anti-VEGF bispecific antibody is CAS Number 1448221-05-3. In certain embodiments, the anti-CD40 antibody is RG7876. In certain embodiments, the cell culture medium is an initial cell culture medium. In certain embodiments, the harvested polypeptide has a trisulfide bond level that is less than the trisulfide bond level of a polypeptide produced under identical conditions, except that the concentration of one or more components differs from the concentration(s) specified above. In certain embodiments, the average % trisulfide in the harvested polypeptide is less than about any one of 20%, 19%, 18%, 17%, 16%, 15%, 14%, 13%, 12%, 10%, 9%, 8%, 7%, 6%, 5%, 4%, 3%, 2%, 1%, 0.5%, or 0.1% (mol trisulfide/mol polypeptide). In certain embodiments, the average trisulfide in the harvested polypeptide is reduced by about any one of 10%, 20%, 25%, 30%, 35%, 40%, 45%, 50%, 55%, 65%, 70%, 75%, 80%, 85%, 90%, 91%, 92%, 93%, 94%, 95%, 96%, 97%, 98%, 99%, or more than 99% relative to the trisulfide bond level of a polypeptide produced under identical conditions, except that the concentration of one or more components differs from the concentration(s) specified above.

In certain embodiments, provided is the use of between about 0 and about 4.5 μM methionine in a cell culture medium for decreasing trisulfide bond levels in a polypeptide selected from the group consisting of: a CEA-IL2v immuocytokine, a FAP-IL2v immunocytokine, an anti-CEA/anti-CD3 bispecific antibody, an anti-VEGF/anti-angiopoietin bispecific antibody, an anti-Ang2/anti-VEGF bispecific antibody, an anti-C5 antibody, and an anti-CD40 antibody. In certain embodiments, the CEA-IL2v immuocytokine is RG7813. In certain embodiments, the FAP-IL2v immunocytokine is RG7461. In certain embodiments, the anti-CEA/anti-CD3 bispecific antibody is RG7802. In certain embodiments, the anti-VEGF/anti-angiopoietin bispecific antibody is RG7716. In certain embodiments, the anti-Ang2/anti-VEGF bispecific antibody is RG7221. In certain embodiments, the anti-Ang2/anti-VEGF bispecific antibody is CAS Number 1448221-05-3. In certain embodiments, the anti-CD40 antibody is RG7876.

In certain embodiments, the basal medium comprises between any one of about 5 μM to about 30 μM, about 10 μM to about 25 μM, or about 15 μM to about 20 μM iron, including any range in between these values. In certain embodiments, the basal medium comprises any one of about 2 μM, 4 μM, 6 μM, 10 μM, 12 μM, 14 μM, 16 μM, 18 μM, 20 μM, 22 μM, 24 μM, 26 μM, 28 μM, 30 μM, 35 μM iron, including any value in between. In certain embodiments, the iron source in the basal medium is any one or combination of the following: iron (II) sulfate, iron (III) sulfate, iron (II) citrate, iron (III) citrate, ammonium iron (II) sulfate hexahydrate, iron (III) sulfate hydrate, ammonium iron (III) sulfate dodecahydrate, iron (II) sulfate heptahydrate, iron (III) nitrate nonahydrate, ammonium iron (III) citrate, iron (III) tartrate, iron (II) lactate hydrate, iron (III) oxalate hexahydrate, iron (II) oxalate dihydrate, iron (III) trifluoroacetylacetonate, iron (II) fumarate, ammonium iron (III) oxalate trihydrate, iron (II) gluconate hydrate, iron (II) D-gluconate dihydrate, (+)-Iron (II) L-ascorbate.

In certain embodiments, the cell culture medium comprises between any one of about 5 μM to about 30 μM, about 10 μM to about 25 μM, or about 15 μM to about 20 μM iron, including any range in between these values. In certain embodiments, the cell culture medium comprises any one of about 2 μM, 4 μM, 6 μM, 10 μM, 12 μM, 14 μM, 16 μM, 18 μM, 20 μM, 22 μM, 24 μM, 26 μM, 28 μM, 30 μM, 35 μM iron, including any value in between. In certain embodiments, the iron source in the cell culture medium is any one or combination of the following: iron (II) sulfate, iron (III) sulfate, iron (II) citrate, iron (III) citrate, ammonium iron (II) sulfate hexahydrate, iron (III) sulfate hydrate, ammonium iron (III) sulfate dodecahydrate, iron (II) sulfate heptahydrate, iron (III) nitrate nonahydrate, ammonium iron (III) citrate, iron (III) tartrate, iron (II) lactate hydrate, iron (III) oxalate hexahydrate, iron (II) oxalate dihydrate, iron (III) trifluoroacetylacetonate, iron (II) fumarate, ammonium iron (III) oxalate trihydrate, iron (II) gluconate hydrate, iron (II) D-gluconate dihydrate, (+)-Iron (II) L-ascorbate. In certain embodiments, the cell culture medium comprises basal medium. In certain embodiments, the cell culture medium comprises basal medium and feed medium (such as batch feed medium). In certain embodiments, the cell culture medium comprises feed medium (such as batch feed medium).

In certain embodiments, the basal medium comprises between any one of about 0.15 μM to about 1.5 μM, about 0.3 μM to about 1.0 μM, or about 0.3 μM to about 0.75 μM vitamin B2, including any range in between these values. In certain embodiments, the basal medium comprises any one of about 0.11 μM, 0.2 μM, 0.4 μM, 0.6 μM, 0.8 μM, 1.0 μM, 1.2 μM, 1.4 μM, 1.6 μM, 1.8 μM, or 2 μM riboflavin (vitamin B2), including any value in between. In certain embodiments, the vitamin B2 source in the basal medium is any one or combination of the following: riboflavin powder (9, D-ribitol 6,7 dimethyl isoalloxazine), riboflavin 5′-monophosphate, or a sodium salt form of a riboflavin 5′-monophosphate.

In certain embodiments, the cell culture medium comprises between any one of about 0.15 μM to about 1.5 μM, about 0.3 μM to about 1.0 μM, or about 0.3 μM to about 0.75 μM vitamin B2, including any range in between these values. In certain embodiments, the cell culture medium comprises any one of about 0.11 μM, 0.2 μM, 0.4 μM, 0.6 μM, 0.8 μM, 1.0 μM, 1.2 μM, 1.4 μM, 1.6 μM, 1.8 μM, or 2 μM riboflavin (vitamin B2), including any value in between. In certain embodiments, the vitamin B2 source in the basal medium is any one or combination of the following: riboflavin powder (9, D-ribitol 6,7 dimethyl isoalloxazine), riboflavin 5′-monophosphate, or a sodium salt form of a riboflavin 5′-monophosphate. In certain embodiments, the cell culture medium comprises basal medium. In certain embodiments, the cell culture medium comprises basal medium and feed medium (such as batch feed medium). In certain embodiments, the cell culture medium comprises feed medium (such as batch feed medium).

In certain embodiments, the basal medium comprises between any one of about 1.5 μM to about 75 μM, about 5 μM to about 50 μM, or about 25 μM to about 40 μM vitamin B6, including any range in between these values. In certain embodiments, the basal medium comprises any one of about 4.5 μM, 5 μM, 10 μM, 15 μM, 20 μM, 25 μM, 30 μM, 35 μM, 40 μM, 45 μM, 50 μM, 55 μM, 60 μM, 65 μM, 70 μM, 75 μM or 80 μM vitamin B6, including any value in between. In certain embodiments, the vitamin B6 source in the basal medium is any one or combination of the following: pyridoxine, pyridoxine monohydrochloride, pyridoxal, pyridoxal monohydrochloride, pyridoxal 5′-phosphate, pyridoxamine, pyridoxamine dihydrochloride, pyridoxamine 5-phosphate, pyritinol, 4-pyridoxic acid.

In certain embodiments, the cell culture medium comprises between any one of about 1.5 μM to about 75 μM, about 5 μM to about 50 μM, or about 25 μM to about 40 μM vitamin B6, including any range in between these values. In certain embodiments, the cell culture medium comprises any one of about 4.5 μM, 5 μM, 10 μM, 15 μM, 20 μM, 25 μM, 30 μM, 35 μM, 40 μM, 45 μM, 50 μM, 55 μM, 60 μM, 65 μM, 70 μM, 75 μM or 80 μM vitamin B6, including any value in between. In certain embodiments, the vitamin B6 source in the cell culture medium is any one or combination of the following: pyridoxine, pyridoxine monohydrochloride, pyridoxal, pyridoxal monohydrochloride, pyridoxal 5′-phosphate, pyridoxamine, pyridoxamine dihydrochloride, pyridoxamine 5-phosphate, pyritinol, 4-pyridoxic acid. In certain embodiments, the cell culture medium comprises basal medium. In certain embodiments, the cell culture medium comprises basal medium and feed medium (such as batch feed medium). In certain embodiments, the cell culture medium comprises feed medium (such as batch feed medium).

In certain embodiments, the basal medium comprises between any one of about 5 μM to about 20 μM, about 7 μM to about 15 μM, or about 10 μM to about 12 μM vitamin B9, including any range in between these values. In certain embodiments, the basal medium comprises any one of about 3.4 μM, 5 μM, 10 μM, 15 μM, 20 μM, or 23 μM vitamin B9, including any value in between. In certain embodiments, the vitamin B9 source in the basal medium is any one or combination of the following: folic acid, folic acid powder, folinic acid calcium salt, tetrahydrofolate, or 4-aminobenzoic acid or para-aminobenzoic acid (PABA).

In certain embodiments, the cell culture medium comprises between any one of about 5 μM to about 20 μM, about 7 μM to about 15 μM, or about 10 μM to about 12 μM vitamin B9, including any range in between these values. In certain embodiments, the cell culture medium comprises any one of about 3.4 μM, 5 μM, 10 μM, 15 μM, 20 μM, or 23 μM vitamin B9, including any value in between. In certain embodiments, the vitamin B9 source in the cell culture medium is any one or combination of the following: folic acid, folinic acid calcium salt, tetrahydrofolate, or 4-aminobenzoic acid or para-aminobenzoic acid (PABA). In certain embodiments, the cell culture medium comprises basal medium. In certain embodiments, the cell culture medium comprises basal medium and feed medium (such as batch feed medium). In certain embodiments, the cell culture medium comprises feed medium (such as batch feed medium).

In certain embodiments, the basal medium comprises between any one of about 0.5 μM to about 2.0 μM, about 1 μM to about 1.7 μM, or about 1.2 μM to about 1.5 μM vitamin B12, including any range in between these values. In certain embodiments, the basal medium comprises any one of about 0.2 μM, 0.4 μM, 0.6 μM, 0.8 μM, 1.0 μM, 1.2 μM, 1.4 μM, 1.6 μM, 1.8 μM, 2.0 μM, 2.2 μM, 2.4 μM, and 2.5 μM vitamin B12, including any value in between. In certain embodiments, the vitamin B12 source in the basal medium is any one or combination of the following: cyanocobalamin and hydroxocobalamin.

In certain embodiments, the cell culture medium comprises between any one of about 0.5 μM to about 2.0 μM, about 1 μM to about 1.7 μM, or about 1.2 μM to about 1.5 μM vitamin B12, including any range in between these values. In certain embodiments, the cell culture medium comprises any one of about 0.2 μM, 0.4 μM, 0.6 μM, 0.8 μM, 1.0 μM, 1.2 μM, 1.4 μM, 1.6 μM, 1.8 μM, 2.0 μM, 2.2 μM, 2.4 μM, and 2.5 μM vitamin B12, including any value in between. In certain embodiments, the vitamin B12 source in the cell culture medium is any one or combination of the following: cyanocobalamin and hydroxocobalamin. In certain embodiments, the cell culture medium comprises basal medium. In certain embodiments, the cell culture medium comprises basal medium and feed medium (such as batch feed medium). In certain embodiments, the cell culture medium comprises feed medium (such as batch feed medium).

In certain embodiments, the basal medium comprises between any one of about 2.0 mM to about 40 mM, about 5 mM to about 30 mM, about 7 mM to about 20 mM, about 8 mM to about 15 mM, about 9.2 mM to about 9.8 mM, about 9.4 mM to about 9.6 mM, or about 9.5 mM hypotaurine, including any range in between these values. In certain embodiments, the basal medium comprises any one of about 2 mM, 4 mM, 6 mM, 8 mM, 9 mM, 9.2 mM, 9.4 mM, 9.6 mM, 9.8 mM, 10 mM, 15 mM, 20 mM, 25 mM, 30 mM, 35 mM, and 40 mM hypotaurine, including any value in between. In certain embodiments, the hypotaurine source in the basal medium is hypotaurine powder.

In certain embodiments, the cell culture medium comprises between any one of about 2.0 mM to about 40 mM, about 5 mM to about 30 mM, about 7 mM to about 20 mM, about 8 mM to about 15 mM, about 9.2 mM to about 9.8 mM, about 9.4 mM to about 9.6 mM, or about 9.5 mM hypotaurine, including any range in between these values. In certain embodiments, the cell culture medium comprises any one of about 2 mM, 4 mM, 6 mM, 8 mM, 9 mM, 9.2 mM, 9.4 mM, 9.6 mM, 9.8 mM, 10 mM, 15 mM, 20 mM, 25 mM, 30 mM, 35 mM, and 40 mM hypotaurine, including any value in between. In certain embodiments, the hypotaurine source in the cell culture medium is hypotaurine powder. In certain embodiments, the cell culture medium comprises basal medium. In certain embodiments, the cell culture medium comprises basal medium and feed medium (such as batch feed medium). In certain embodiments, the cell culture medium comprises feed medium (such as batch feed medium).

In certain embodiments, the basal medium comprises between any one of about 0.5 mM to about 1.5 mM, about 0.75 mM to about 1.25 mM, or about 1.0 mM methionine, including any range in between these values. In certain embodiments, the basal medium comprises any one of about 0 mM, 0.25 mM, 0.5 mM, 0.75 mM, 1.0 mM, 1.25 mM, 1.5 mM, or 1.58 mM methionine, including any value in between. In certain embodiments, the methionine source in the basal medium is any one or combination of the following: methionine powder, L-methionine, DL-methionine, L-methionine hydrochloride solution, N-acetyl-L-methionine, N-acetyl-D,L-Methionine, L-methionine methyl ester hydrochloride, S-(5′-adenosyl)-L-methionine chloride dihydrochloride, and S-(5′-Adenosyl)-L-methionine iodide.

In certain embodiments, the cell culture medium comprises between any one of about 0.5 mM to about 4.0 mM, about 1.5 mM to about 3 mM, or about 2 mM to about 2.5 mM methionine, including any range in between these values. In certain embodiments, the cell culture medium comprises any one of about 0 mM, 0.25 mM, 0.5 mM, 0.75 mM, 1.0 mM, 1.25 mM, 1.5 mM, 1.75 mM, 2.0 mM, 2.25 mM, 2.5 mM, 2.75 mM, 3.0 mM, 3.25 mM, 3.5 mM, 3.75 mM, 4.0 mM, 4.25 mM, or 4.5 mM methionine, including any value in between. In certain embodiments, the methionine source in the cell culture medium is any one or combination of the following: methionine powder, L-methionine, DL-methionine, L-methionine hydrochloride solution, N-acetyl-L-methionine, N-acetyl-D,L-Methionine, L-methionine methyl ester hydrochloride, S-(5′-adenosyl)-L-methionine chloride dihydrochloride, and S-(5′-adenosyl)-L-methionine iodide. In certain embodiments, the cell culture medium comprises basal medium. In certain embodiments, the cell culture medium comprises basal medium and feed medium (such as batch feed medium). In certain embodiments, the cell culture medium comprises feed medium (such as batch feed medium).

In certain embodiments, the basal medium lacks cystine.

In certain embodiments, the basal medium contains between about 1.4 mM to about 3.0 mM cysteine or cystine (such as any one of about 1.4 mM, 1.6 mM, 1.8 mM, 2.0 mM, 2.2 mM, 2.4 mM, 2.6 mM, 2.8 mM, or 3.0 mM cysteine or cystine, including any value in between). In certain embodiments, the cysteine source in the basal medium is any one or combination of the following: L-cysteine and L-cysteine monohydrochloride monohydrate powder. In certain embodiments, the cystine source in the basal medium is cystine disodium salt monohydrate powder.

In certain embodiments, the cell culture medium contains between about 1.4 mM to about 3.0 mM cysteine or cystine (such as any one of about 1.4 mM, 1.6 mM, 1.8 mM, 2.0 mM, 2.2 mM, 2.4 mM, 2.6 mM, 2.8 mM, or 3.0 mM cysteine or cystine, including any value in between). In certain embodiments, the cysteine source in the cell culture medium is any one or combination of the following: L-cysteine and L-cysteine monohydrochloride monohydrate powder. In certain embodiments, the cystine source in the cell culture medium is cystine disodium salt monohydrate powder. In certain embodiments, the cell culture medium comprises basal medium. In certain embodiments, the cell culture medium comprises basal medium and feed medium (such as batch feed medium). In certain embodiments, the cell culture medium comprises feed medium (such as batch feed medium).

In certain embodiments, the basal medium comprises between about 0 mM to about 1.58 mM methionine (such as any one of about 0 mM, 0.25 mM, 0.5 mM, 0.75 mM, 1 mM, 1.25 mM, or 1.58 mM methionine, including any value in between). In certain embodiments, the basal medium comprises about 0 mM to about 3.0 mM cysteine (such as any one of about 0 mM, 0.2 mM, 0.4 mM, 0.6 mM, 0.8 mM, 1.0 mM, 1.2 mM, 1.4 mM, 1.6 mM, 1.8 mM, 2.0 mM, 2.2 mM, 2.4 mM, 2.6 mM, 2.8 mM, or 3.0 mM cysteine, including any value in between). In certain embodiments, the basal medium comprises between about 0 mM to about 1.58 mM methionine (such as any one of about 0 mM, 0.25 mM, 0.5 mM, 0.75 mM, 1 mM, 1.25 mM, or 1.58 mM methionine, including any value in between) and about 0 mM to about 3.0 mM cysteine (such as any one of about 0 mM, 0.2 mM, 0.4 mM, 0.6 mM, 0.8 mM, 1.0 mM, 1.2 mM, 1.4 mM, 1.6 mM, 1.8 mM, 2.0 mM, 2.2 mM, 2.4 mM, 2.6 mM, 2.8 mM, or 3.0 mM cysteine, including any value in between).

In certain embodiments, the cell culture medium comprises between about 0 mM to about 1.58 mM methionine (such as any one of about 0 mM, 0.25 mM, 0.5 mM, 0.75 mM, 1 mM, 1.25 mM, or 1.58 mM methionine, including any value in between). In certain embodiments, the cell culture comprises about 0 mM to about 3.0 mM cysteine (such as any one of about 0 mM, 0.2 mM, 0.4 mM, 0.6 mM, 0.8 mM, 1.0 mM, 1.2 mM, 1.4 mM, 1.6 mM, 1.8 mM, 2.0 mM, 2.2 mM, 2.4 mM, 2.6 mM, 2.8 mM, or 3.0 mM cysteine, including any value in between). In certain embodiments, the cell culture medium comprises between about 0 mM to about 1.58 mM methionine (such as any one of about 0 mM, 0.25 mM, 0.5 mM, 0.75 mM, 1 mM, 1.25 mM, or 1.58 mM methionine, including any value in between) and about 0 mM to about 3.0 mM cysteine (such as any one of about 0 mM, 0.2 mM, 0.4 mM, 0.6 mM, 0.8 mM, 1.0 mM, 1.2 mM, 1.4 mM, 1.6 mM, 1.8 mM, 2.0 mM, 2.2 mM, 2.4 mM, 2.6 mM, 2.8 mM, or 3.0 mM cysteine, including any value in between). In certain embodiments, the cell culture medium comprises basal medium. In certain embodiments, the cell culture medium comprises basal medium and feed medium (such as batch feed medium). In certain embodiments, the cell culture medium comprises feed medium (such as batch feed medium).

In certain embodiments, the method comprises adding a concentrated nutrient mixture (“batch feed”) to the host cell culture in one or more increments. In certain embodiments, the batch feed medium lacks iron (Fe, such as Fe (II) and/or Fe (III)). In certain embodiments, the batch feed lacks one or more of the following: riboflavin (vitamin B2), pyridoxine (vitamin B6), pyridoxal (vitamin B6), folate/folic acid (vitamin B9) and cyanocobalmin (vitamin B12). In certain embodiments the batch feed lacks riboflavin (vitamin B2), pyridoxine (vitamin B6), pyridoxal (vitamin B6), folic acid (vitamin B9) and cyanocobalmin (vitamin B120). In certain embodiments, the batch feed lacks iron (Fe, such as Fe (II) and/or Fe (III) and one or more of the following: riboflavin (vitamin B2), pyridoxine (vitamin B6), pyridoxal (vitamin B6), folic acid (vitamin B9) and cyanocobalmin (vitamin B12). In certain embodiments, the batch feed lacks iron (Fe, such as Fe (II) and/or Fe (III) riboflavin (vitamin B2), pyridoxine (vitamin B6), pyridoxal (vitamin B6), folic acid (vitamin B9) and cyanocobalmin (vitamin B12). In certain embodiments, the batch feed medium lacks cystine. In certain embodiments, the batch feed medium lacks cysteine. In certain embodiments, the batch feed medium lacks methionine. In certain embodiments, the batch feed medium lacks cysteine and methionine. In certain embodiments, the batch feed medium lacks cysteine, cystine, and methionine.

In certain embodiments, the method further comprises supplementing the culture or cell culture fluid of said host cell, the pre-harvest cell culture fluid (PHCCF) of said host cell, or a harvested cell culture fluid (HCCF) of said host cell with a chelating agent and a reducing agent.

In certain embodiments, provided herein is a method for decreasing the level of trisulfide bonds in a polypeptide produced by a host cell, comprising supplementing a culture or cell culture fluid of said host cell, a pre-harvest cell culture fluid (PHCCF) of said host cell, or a harvested cell culture fluid (HCCF) of said host cell with a chelating agent and a reducing agent, whereby the level of trisulfide bonds in the polypeptide is reduced.

In certain embodiments, the chelating agent and the reducing agent are added to the culture at any one of about 4.5 hours, 4.0 hours, 3.5 hours, 3.0 hours, 2.5 hours, 2.0 hours, 1.5 hours, 1.0 hours or 0.5 hours before harvest, including any value in between. In certain embodiments, the chelating agent and the reducing agent are added to the culture at harvest.

In certain embodiments, the chelating agent is added to the culture, cell culture fluid, PHCCF, or HCCF of said host cell prior to the reducing agent. In certain embodiments, the chelating agent is added to the culture, cell culture fluid, PHCCF, or HCCF of said host cell between any one of about 60 minutes, 55 minutes, 50 minutes, 45 minutes, 40 minutes, 35 minutes, or 30 minutes prior to adding the chelating agent, including any value in between.

In certain embodiments, the reducing agent is added to the culture, cell culture fluid, PHCCF, or HCCF of said host cell prior to the chelating agent.

In certain embodiments, the chelating agent and the reducing agent are added to the culture, cell culture fluid, PHCCF, or HCCF of said host cell simultaneously.

In certain embodiments, the chelating agent and the reducing agent are added to the culture, the cell culture fluid, the PHCCF or the HCCF, and the culture, the cell culture fluid, the PHCCF, or the HCCF is maintained at a temperature of any one of about 15° C., 16° C., 17° C., 18° C., 19° C., 20° C., 21° C., 22° C., 23° C., 24° C., 2 5° C., 26° C., 27° C., 28° C., 29° C., 30° C., 31° C., 32° C., 33° C., 34° C., 35° C., 36° C., or 37° C., including any value in between. In certain embodiments, the chelating agent and the reducing agent are added to the culture, the cell culture fluid, the PHCCF, or the HCCF, and the culture, the cell culture fluid, the PHCCF, or the HCCF is maintained at a pH of any one of about 6.5, 6.6, 6.7, 6.8, 6.9, 7.0, 7.1, 7.2, 7.3, 7.4, or 7.5, including any value in between. In certain embodiments, the chelating agent and the reducing agent are added to the culture, the cell culture fluid, the PHCCF, or the HCCF, and the culture, the cell culture fluid, the PHCCF or the HCCF is maintained at % DO (dissolved oxygen) of any one of about 5%, 6%, 7%, 8%, 9%, 10%, 11%, 12%, 13%, 14%, 15%, 16%, 17%, 18%, 19%, 20%, 25%, 26%, 27%, 28%, 29%, or 30%, including any value in between. In certain embodiments, the HCCF is maintained at % DO (dissolved oxygen) of more than about 30%, including any one of about 31%, 35%, 40%, 45%, 50%, 55%, 60%, 65%, 70%, 75%, 80%, 85%, 90%, 95%, 99%, or 100%, including any value in between. In certain embodiments, the chelating agent and the reducing agent are added to the culture, the cell culture fluid, or the PHCCF, and the culture, the cell culture fluid, or the PHCCF is maintained at a temperature of any one of about 15° C., 16° C., 17° C., 18° C., 19° C., 20° C., 21° C., 22° C., 23° C., 24° C., 2 5° C., 26° C., 27° C., 28° C., 29° C., 30° C., 31° C., 32° C., 33° C., 34° C., 35° C., 36° C., or 37° C., including any value in between, at a pH of any one of about 6.5, 6.6, 6.7, 6.8, 6.9, 7.0, 7.1, 7.2, 7.3, 7.4, or 7.5, including any value in between, and at a % DO (dissolved oxygen) of any one of about 5%, 6%, 7%, 8%, 9%, 10%, 11%, 12%, 13%, 14%, 15%, 16%, 17%, 18%, 19%, 20%, 25%, 26%, 27%, 28%, 29%, or 30%, including any value in between. In certain embodiments, the chelating agent and the reducing agent are added to the HCCF, and the HCCF is maintained at a temperature of any one of about 15° C., 16° C., 17° C., 18° C., 19° C., 20° C., 21° C., 22° C., 23° C., 24° C., 2 5° C., 26° C., 27° C., 28° C., 29° C., 30° C., 31° C., 32° C., 33° C., 34° C., 35° C., 36° C., or 37° C., including any value in between, at a pH of any one of about 6.5, 6.6, 6.7, 6.8, 6.9, 7.0, 7.1, 7.2, 7.3, 7.4, or 7.5, including any value in between, and at a % DO (dissolved oxygen) of any one of about 5%, 6%, 7%, 8%, 9%, 10%, 11%, 12%, 13%, 14%, 15%, 16%, 17%, 18%, 19%, 20%, 25%, 26%, 27%, 28%, 29%, 30%, 35%, 40%, 45%, 50%, 55%, 60%, 65%, 70%, 75%, 80%, 85%, 90%, 95%, 99%, and 100% including any value in between.

In certain embodiments, the chelating agent is any one or combination of the following: ethylenediaminetetraacetic acid (EDTA), nitrilotriacetic acid (NTA), ethylenediamine-N,N′-disuccinic acid (EDDS), citrate, oxalate, tartrate, ethylene-bis(oxyethylenenitrilo)tetraacetic acid (EGTA), diethylenetriaminepentaacetic acid (DTPA), 5-sulfosalicylic acid, N,N-dimethyldodecylamine N-oxide, dithiooxamide, ethylenediamine, salicylaldoxime, N-(2′-hydroxyethyl)iminodiacetic acid (HIMDA), oxine quinolinol, and sulphoxine. In certain embodiments, the chelating agent is selected from the group consisting of ethylenediaminetetraacetic acid (EDTA), nitrilotriacetic acid (NTA), ethylenediamine-N,N′-disuccinic acid (EDDS), and citrate. In certain embodiments, the ethylenediaminetetraacetic acid (EDTA), nitrilotriacetic acid (NTA), ethylenediamine-N,N′-disuccinic acid (EDDS), or citrate is added to the culture, the cell culture fluid, the PHCCF, or the HCCF of said host cell to achieve a final concentration of 20 mM.

In certain embodiments, the reducing agent is any one or combination of the following: glutathione (GSH), L-glutathione (L-GSH), cysteine, L-cysteine, tris(2-carboxyethyl)phosphine hydrochloride (TCEP), 2,3-tert-butyl-4-hydroxyanisole, 2,6-di-tert-butyl-4-methylphenol, 3-aminopropane-l-sulfonic acid, adenosylhomocysteine, anserine, B-alanine, B-carotene, butylated hydroxyanisole, butylated hydroxytoluene, carnosine, carvedilol, curcumin, cysteamine, cysteamine hydrochloride, dexamethasone, diallyldisulfide, DL-lanthionine, DL-thiorphan, ethoxyquin, gallic acid, gentisic acid sodium salt hydrate, glutathione disulfide, glutathione reduced ethyl ester, glycine, hydrocortisone, hypotaurine, isethionic acid ammonium salt, L-cysteine-glutathione Disulfide, L-cysteinesulfinic acid monohydrate, lipoic acid, reduced lipoic acid, mercaptopropionyl glycine, methionine, methylenebis(3-thiopropionic acid), oxalic acid, quercitrin hydrate, resveratrol, retinoic acid, S-carboxymethyl-L-cysteine, selenium, selenomethionine, silver diethyldithiocarbamate, taurine, thiolactic acid, tricine, vitamin C, vitamin E, vitamin B1, vitamin B2, vitamin B3, vitamin B4, vitamin B5, vitamin B6, and vitamin B11. In certain embodiments, the reducing agent is selected from the group consisting of: cysteine and L-cysteine. In certain embodiments, the cysteine or L-cysteine is added to the culture, the cell culture fluid, the PHCCF, or the HCCF of said host cell to achieve a final concentration of any one of about 3.0 mM, 3.5 mM, 4.0 mM, 4.5 mM, 5.0 mM, 5.5 mM, or 6 mM, including any value in between.

Any cell culture medium known in the art, suitable for the desired type of cell and/or polypeptide product, may be used in a method described herein. In some embodiments, the cell culture medium is a chemically defined medium. In other embodiments, the cell culture medium is a chemically undefined medium.

Commercially available media may be used, including such as, but not limited to, Ham's F10 (Sigma), Minimal Essential Medium ([MEM], Sigma), RPMI-1640 (Sigma), Dulbecco's Modified Eagle's Medium ([DMEM], Sigma), and any of these media may be modified as described herein. In addition, any of the media described in Ham and Wallace, Meth. Enz., 58:44 (1979), Barnes and Sato, Anal. Biochem., 102:255 (1980), Vijayasankaran et al., Biomacromolecules., 6:605:611 (2005), Patkar et al., J Biotechnology, 93:217-229 (2002), U.S. Pat. Nos. 4,767,704; 4,657,866; 4,927,762; or 4,560,655; WO 90/03430; WO 87/00195; U.S. Pat. No. Re. 30,985; or U.S. Pat. No. 5,122,469, the disclosures of all of which are incorporated herein by reference in their entirety, may be modified as detailed herein.

Any media provided herein may also be supplemented as necessary with hormones and/or other growth factors (such as insulin, transferrin, or epidermal growth factor), ions (such as sodium, chloride, calcium, magnesium, and phosphate), buffers (such as HEPES), nucleosides (such as adenosine and thymidine), and glucose or an equivalent energy source. In certain embodiments, the cell culture medium used in the methods provided herein is a chemically defined cell culture medium. In certain embodiments, the cell culture medium used in the methods provided herein is a chemically undefined cell culture medium. In certain embodiments, the cell culture medium used in the methods provided herein contains proteins derived from a plant or an animal. In certain embodiments, the cell culture medium used in the methods provided herein is free of proteins derived from a plant or an animal. Any other necessary supplements may also be included at appropriate concentrations that would be known to those skilled in the art.

Any cell culture technique known in the art may be used with a method as described herein. Examples of cell culture techniques include, but are not limited to, single cell culturing passaging, extended cell culturing passaging, a seed or inoculum train, concentrated feed supplementation, cell bank generation, perfusion culturing, and fed-batch culturing.

In certain embodiments, the polypeptide is secreted into the cell culture medium. In certain embodiments, the methods provided herein further comprise the step of recovering the polypeptide from the cell culture medium.

In certain embodiments, the methods provided herein further comprise measuring the level of trisulfide bonds in the polypeptide. The presence of trisulfide bonds can be detected using any of a number of methods, including methods described in the Examples and methods known to those of ordinary skill in the art. For example, trisulfide bonds can be detected using peptide mapping and can be detected based on an increase in mass of the intact protein due to an extra sulfur atom (32 Da). In certain embodiments, trisulfide bonds can be detected using mass spectrum, or by high pressure liquid chromatography and mass spectrometry (peptide mapping utilizing a LC-MS system). In certain embodiments, trisulfide bonds can be detected through peptide mapping wherein select peptides derived from the intact molecule, including those containing sulfide bonds, are analyzed by LC-MS. In certain embodiments, trisulfide bonds can also be detected indirectly, e.g. by assessing molecular folding or thermal stability. In certain embodiments, the presence of trisulfide bonds in antibodies can be detected or identified as a result of increased sensitivity to heat treatment, for example as demonstrated by an increased level of fragmentation following sample preparation for non-reducing electrophoresis (see, e.g., US 2012/0264916). In certain embodiments, trisulfide bonds can be detected via hydrophobic interaction liquid chromatography combined with charged aerosol detection (HILIC-CAD), according to the method described in Zhang et al. (2010) Journal of Chromatography A. 1217, 5776-5784. In certain embodiments, the average trisulfide bond level in a polypeptide produced according to any one or combination of methods provided herein is less than about any one of 20%, 19%, 18%, 17%, 16%, 15%, 14%, 13%, 12%, 10%, 9%, 8%, 7%, 6%, 5%, 4%, 3%, 2%, 1%, 0.5%, or 0.1% (mol trisulfide/mol polypeptide).

In a related aspect, provided is a polypeptide produced according to a method herein. Such polypeptides are described in further detail below.

### Methods of Producing and Purifying Polypeptides

The methods provided herein can be used to produce polypeptides, including, e.g., antibodies and bispecific antibodies, in any type of animal cell, such as a recombinant animal cell. The term “animal cells” encompasses invertebrate, non-mammalian vertebrate (e.g., avian, reptile and amphibian) and mammalian cells. Examples of invertebrate cells include the following insect cells: Spodoptera frugiperda (caterpillar), Aedes aegypti (mosquito), Aedes albopictus (mosquito), Drosophila melanogaster (fruit fly), and Bombyx mori (See, e.g., Luckow et al., Bio/Technology, 6:47-55 (1988); Miller et al., in Genetic Engineering, Setlow, J. K. et al., eds., Vol. 8 (Plenum Publishing, 1986), pp. 277-279; and Maeda et al., Nature, 315:592- 594 (1985)).

In certain embodiments, the cells are mammalian cells. Mammalian cell lines available as hosts for expression are well known in the art and include, but are not limited to, immortalized cell lines available from the American Type Culture Collection (ATCC) and any cell lines used in an expression system known in the art can be used to produce polypeptides (such as antibodies or bispecific antibodies) according to the methods provided herein. Examples of mammalian cells include human retinoblasts (PER.C6 (CruCell, Leiden, The Netherlands)); monkey kidney CV1 line transformed by SV40 (COS-7, ATCC CRL 1651(Gluzman et al., 1981, Cell 23:175)); human embryonic kidney line (293, 293 EBNA, MSR 293, or 293 cells subcloned for growth in suspension culture, Graham et al., J. Gen Virol., 36:59 (1977)); baby hamster kidney cells (BHK, ATCC CCL 10); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)) mouse L cells; 3T3 cells (ATCC CCL 163); monkey kidney cells (CVI ATCC CCL 70); African green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; a human hepatoma line (Hep G2); human epidermal A431 cells, human Colo205 cells, other transformed primate cell lines, normal diploid cells, cell strains derived from in vitro culture of primary tissue, primary explants, HL-60, U937, HaK or Jurkat cells. Optionally, mammalian cell lines such as HepG2/3B, KB, NIH 3T3 or S49, for example, can be used for expression of the polypeptide when it is desirable to use the polypeptide in various signal transduction or reporter assays. In certain embodiments, the mammalian cell is a CHO cell or a derivative thereof, such as Veggie CHO and related cell lines which grow in serum-free media (Rasmussen et al., 1998, Cytotechnology 28: 31).

The invention is also applicable to hybridoma cells. The term “hybridoma” refers to a hybrid cell line produced by the fusion of an immortal cell line of immunologic origin and an antibody producing cell. The term encompasses progeny of heterohybrid myeloma fusions, which are the result of a fusion with human cells and a murine myeloma cell line subsequently fused with a plasma cell, commonly known as a trioma cell line. Furthermore, the term is meant to include any immortalized hybrid cell line that produces antibodies such as, for example, quadromas (See, e.g., Milstein et al., Nature, 537:3053 (1983)). The hybrid cell lines can be of any species, including human and mouse.

In certain embodiments, the mammalian cell is a non-hybridoma mammalian cell that has been transformed with exogenous isolated nucleic acid encoding a polypeptide of interest, including in especially preferred embodiments, nucleic acids encoding antibodies (such as bispecific antibodies), antibody fragments, such as ligand-binding fragments, and chimeric antibodies. By “exogenous nucleic acid” or “heterologous nucleic acid” is meant a nucleic acid sequence that is foreign to the cell, or homologous to the cell but in a position within the host cell nucleic acid in which the nucleic acid is ordinarily not found.

An isolated nucleic acid is a nucleic acid molecule that is identified and separated from at least one contaminant nucleic acid molecule with which it is ordinarily associated in the natural source of the polypeptide nucleic acid. An isolated nucleic acid molecule is other than in the form or setting in which it is found in nature. An isolated nucleic acid is preferably a non-chromosomal nucleic acid, i.e. isolated from the chromosomal environment in which it naturally exists. Isolated nucleic acid molecules therefore are distinguished from the nucleic acid molecule as it exists in natural cells. However, an isolated nucleic acid molecule includes a nucleic acid molecule contained in cells that ordinarily express the polypeptide where, for example, the nucleic acid molecule is in a chromosomal location different from that of natural cells.

The polypeptide of interest preferably is recovered from the culture medium as a secreted polypeptide, although it also may be recovered from host cell lysates when directly expressed without a secretory signal. As a first step, the culture medium or lysate is centrifuged to remove particulate cell debris. The polypeptide thereafter is purified from contaminant soluble proteins and polypeptides, with the following procedures being exemplary of suitable purification procedures: by fractionation on immunoaffinity or ion-exchange columns; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; and protein A Sepharose columns to remove contaminants such as IgG. A protease inhibitor such as phenyl methyl sulfonyl fluoride (PMSF) also may be useful to inhibit proteolytic degradation during purification. One skilled in the art will appreciate that purification methods suitable for the polypeptide of interest may require modification to account for changes in the character of the polypeptide upon expression in recombinant cell culture.

### Exemplary Polypeptides

Various polypeptides may be produced according to the methods provided herein. Examples include, but are not limited to, e.g., growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-dotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; Protein A or D; rheumatoid factors; a neurotrophic factor such as bone derived neurotrophic factor (BDNF), neurotrophin-3, -4, -5, or -6 (NT-3, NT-4, NT-5, or NT-6), or a nerve growth factor such as NGF-˜; platelet-derived growth factor (PDGF); fibroblast growth factor such as αFGF and bFGF; epidermal growth factor (EGF); transforming growth factor (TGF) such as TGF-alpha and TGF-beta, including TGF-betal, TGF-beta2, TGF-beta3, TGF-beta4,or TGF-beta5; insulin-like growth factor-I and -II (IGF-I and IGF-II); des(1-3)-IGF-1 (brain IGF-1), insulin-like growth factor binding proteins; CD proteins such as CD3, CD4, CD8. CD19, CD20, CD34, and CD40; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-17; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD 11a, CD 11 b, CD 11c, CD 18, an ICAM, VLA-4 and VCAM; a tumor associated antigen such as HER2, HER3 or HERA receptor; and fragments of any of the above-listed polypeptides.

### Antibody Production and Purification

In some embodiments, polypeptide produced according to a method provided herein is an antibody or a fragment thereof. In some embodiments, the antibody produced by a method described herein is a humanized antibody, a chimeric antibody, a human antibody, a library-derived antibody, or a multispecific antibody (such as a bispecific antibody). In certain embodiments, the antibody fragment produced by a method provided herein is a Fab, a Fab′, an F(ab′)₂, an scFv, an (scFv)₂, a dAb, a complementarity determining region (CDR) fragment, a linear antibody, a single-chain antibody molecule, a minibody, a diabody, and multispecific antibody formed from antibody fragments.

Antibodies may be produced using recombinant methods, for example in the production of an antibody using mammalian cells (e.g., CHO cells). For recombinant production of an antibody, nucleic acid encoding the antibody is isolated and inserted into a replicable vector for further cloning (amplification of the DNA) or for expression. DNA encoding the antibody may be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

An antibody may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (e.g., cleaved by a signal peptidase) by the host cell. In mammalian cell expression, mammalian signal sequences as well as viral secretory leaders, for example, the herpes simplex gD signal, are available.

An antibody may be produced intracellularly or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems may be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

Standard protein purification methods known in the art can be employed to obtain substantially homogeneous preparations of an antibody produced according to a method provided herein. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

Additionally or alternatively, antibodies can be purified using, for example, hydroxylapatite chromatography, hydrophobic interaction chromatography, gel electrophoresis, dialysis, and affinity chromatography, with affinity chromatography being among one of the typically preferred purification steps. In certain aspects, the preparation derived from the cell culture medium as described above is applied onto the Protein A immobilized solid phase to allow specific binding of the multispecific antigen-binding protein of interest to Protein A. The solid phase is then washed to remove contaminants non-specifically bound to the solid phase. The multispecific antigen-binding protein (such as a bispecific antibody) is recovered from the solid phase by elution into a solution containing a chaotropic agent or mild detergent. Exemplary chaotropic agents and mild detergents include, but are not limited to, Guanidine-HCl, urea, lithium perclorate, Arginine, Histidine, SDS (sodium dodecyl sulfate), Tween, Triton, and NP-40, all of which are commercially available.

The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising an antibody (such as an antibody produced according to a method provided herein) and contaminants may be subjected to low pH hydrophobic interaction chromatography using an elution buffer at a pH between about 2.5-4.5, preferably performed at low salt concentrations (e.g., from about 0-0.25M salt). The production of an antibody can alternatively or additionally (to any of the foregoing particular methods) comprise dialyzing a solution comprising a mixture of the polypeptides.

In some embodiments, an antibody described herein is an antigen-binding fragment thereof. Examples of antigen-binding fragment include Fab, Fab′, F(ab′)₂, and Fv fragments; diabodies; linear antibodies; single-chain antibody molecules; and multispecific antibodies formed from antibody fragments. The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known. “Fv” is the minimum antibody fragment which contains a complete antigen-binding site. “Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, in The Pharmacology ofMonoclonal Antibodies, vol. 113, Rosenburg and Moore eds., (Springer-Verlag, New York, 1994), pp. 269-315. Many of the methods for purifying an antibody described above may be suitably adapted for purifying an antigen-binding antibody fragment.

In certain embodiments, the cell cultured in a cell culture medium of the present disclosure is used to produce a bispecific antibody. In certain embodiments, the bispecific antibody is composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation (see WO 94/04690). For further details of generating bispecific antibodies see, for example, Suresh et al., Methods in Enzymology, 121:210 (1986). According to another approach, the interface between a pair of antibody molecules can be engineered to maximize the percentage of heterodimers which are recovered from recombinant cell culture (see W096/27011). The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers. Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (see U.S. Pat. No. 4,676,980), and for treatment of HIV infection (see WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques. Antibodies with more than two valencies are contemplated. For example, trispecific antibodies can be prepared (see Tuft et al. J. Immunol. 147: 60 (1991)).

### Target Molecules

Examples of molecules that may be targeted by an antibody (or multispecific antibody, such as a bispecific antibody) produced according to a method provided herein include, but are not limited to, soluble serum proteins and their receptors and other membrane bound proteins (e.g., adhesins). In another embodiment, a multispecific antigen-binding protein provided herein is capable of binding one, two or more cytokines, cytokine-related proteins, and cytokine receptors selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (αFGF), FGF2 ((βFGF), FGF3 (int-2), FGF4 (HST), FGFS, FGF6 (HST-2), FGF7 (KGF), FGF9, FGF10, FGF11, FGF12, FGF12B, FGF14, FGF16, FGF17, FGF19, FGF20, FGF21, FGF23, IGF1, IGF2, IFNA1, IFNA2, IFNA4, IFNA5, IFNA6, IFNA7, IFN81, IFNG, IFNWI, FEL1, FEL1 (EPSELON), FEL1 (ZETA), IL 1A, IL 1B, IL2, IL3, IL4, IL5, IL6, IL7, IL8, IL9, IL10, IL 11, IL 12A, IL 12B, IL 13, IL 14, IL 15, IL 16, IL 17, IL 17B, IL 18, IL 19, IL20, IL22, IL23, IL24, IL25, IL26, IL27, IL28A, IL28B, IL29, IL30, PDGFA, PDGFB, TGFA, TGFB1, TGFB2, TGFBb3, LTA (TNF-β), LTB, TNF (TNF-α), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL 11RA, IL12RB1, IL12RB2, IL13RA1, IL13RA2, IL15RA, IL17R, IL18R1, IL20RA, IL21R, IL22R, IL1HY1, IL1RAP, IL1RAPL1, IL1RAPL2, IL1RN, IL6ST, IL18BP, IL18RAP, IL22RA2, AIF1, HGF, LEP (leptin), PTN, and THPO.

In certain embodiments, the methods provided herein can be used to produce an antibody (or a multispecific antibody, such as a bispecific antibody) to a chemokine, chemokine receptor, or a chemokine-related protein selected from the group consisting of CCLI (1-309), CCL2 (MCP -1/MCAF), CCL3 (MIP-Iα), CCL4 (MIP-Iβ), CCL5 (RANTES), CCL7 (MCP-3), CCL8 (mcp-2), CCL11 (eotaxin), CCL 13 (MCP-4), CCL 15 (MIP-Iδ), CCL 16 (HCC-4), CCL 17 (TARC), CCL 18 (PARC), CCL 19 (MDP-3b), CCL20 (MIP-3α), CCL21 (SLC/exodus-2), CCL22 (MDC/STC-1), CCL23 (MPIF-1), CCL24 (MPIF-2 /eotaxin-2), CCL25 (TECK), CCL26 (eotaxin-3), CCL27 (CTACK/ILC), CCL28, CXCLI (GROI), CXCL2 (GRO2), CXCL3 (GRO3), CXCL5 (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-Iβ), BLRI (MDR15), CCBP2 (D6/JAB61), CCRI (CKRI/HM145), CCR2 (mcp-IRB IRA), CCR3 (CKR3/CMKBR3), CCR4, CCR5 (CMKBR5/ChemR13), CCR6 (CMKBR6/CKR-L3/STRL22/DRY6), CCR7 (CKR7/EBII), CCR8 (CMKBR8/TER1/CKR- L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Rα), IL8RB (IL8Rβ), LTB4R (GPR16), TCP10, CKLFSF2, CKLFSF3, CKLFSF4, CKLFSF5, CKLFSF6, CKLFSF7, CKLFSF8, BDNF, C5, C5R1, CSF3, GRCC10 (C10), EPO, FY (DARC), GDF5, HDF1, HDF1α, DL8, PRL, RGS3, RGS13, SDF2, SLIT2, TLR2, TLR4, TREM1, TREM2, and VHL.

In another embodiment the antibody or bispecific antibody produced according to a method provided herein is capable of binding one or more targets selected from the following: 0772P (CA125, MUC16) (i.e., ovarian cancer antigen), ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; amyloid beta; ANGPTL; ANGPT2; ANGPTL3; ANGPTL4; ANPEP; APC; APOC1; AR; ASLG659; ASPHD1 (aspartate beta-hydroxylase domain containing 1; LOC253982); AZGP1 (zinc-a-glycoprotein); B7.1; B7.2; BAD; BAFF-R (B cell -activating factor receptor, BLyS receptor 3, BR3; BAG1; BAI1; BCL2; BCL6; BDNF; BLNK; BLRI (MDR15); BMP1; BMP2; BMP3B (GDF10); BMP4; BMP6; BMP8; BMPR1A; BMPR1B (bone morphogenic protein receptor-type IB); BMPR2; BPAG1 (plectin); BRCA1; Brevican; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP1δ); CCL16 (HCC-4); CCL17 (TARC); CCL18 (PARC); CCL19 (MIP-3β); CCL2 (MCP-1); MCAF; CCL20 (MIP-3α); CCL21 (MTP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MTP-Iα); CCL4 (MDP-Iβ); CCL5(RANTES); CCL7 (MCP-3); CCL8 (mcp-2); CCNA1; CCNA2; CCND1; CCNE1; CCNE2; CCR1 (CKRI/HM145); CCR2 (mcp-IRβ/RA);CCR3 (CKR/CMKBR3); CCR4; CCR5 (CMKBR5/ChemR13); CCR6 (CMKBR6/CKR-L3/STRL22/DRY6); CCR7 (CKBR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD22 (B-cell receptor CD22-B isoform); CD24; CD28; CD3; CD37; CD38; CD3E; CD3G; CD3Z; CD4; CD40; CD40L; CD44; CD45RB; CD52; CD69; CD72; CD74; CD79A (CD79α, immunoglobulin-associated alpha, a B cell-specific protein); CD79B; CDS; CD80; CD81; CD83; CD86; CDH1 (E-cadherin); CDH10; CDH12; CDH13; CDH18; CDH19; CDH2O; CDH5; CDH7; CDH8; CDH9; CDK2; CDK3; CDK4; CDK5; CDK6; CDK7; CDK9; CDKN1A (p21/WAF1/Cip1); CDKN1B (p27/Kip1); CDKN1C; CDKN2A (P16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3;CLDN7 (claudin-7); CLL-1 (CLEC12A, MICL, and DCAL2); CLN3; CLU (clusterin); CMKLR1; CMKOR1 (RDC1); CNR1; COL 18A1; COL1A1; COL4A3; COL6A1; complement factor D; CR2; CRP; CRIPTO (CR, CR1, CRGF, CRIPTO, TDGF1, teratocarcinoma-derived growth factor); CSFI (M-CSF); CSF2 (GM-CSF); CSF3 (GCSF); CTLA4; CTNNB1 (b-catenin); CTSB (cathepsin B); CX3CL1 (SCYDI); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR5 (Burkitt's lymphoma receptor 1, a G protein-coupled receptor); CXCR6 (TYMSTR/STRL33/Bonzo); CYB5; CYC1; CYSLTR1; DAB2IP; DES; DKFZp451J0118; DNCLI; DPP4; E16 (LAT1, SLC7A5); E2F1; ECGF1; EDG1; EFNA1; EFNA3; EFNB2; EGF; EGFR; ELAC2; ENG; ENO1; ENO2; ENO3; EPHB4; EphB2R; EPO; ERBB2 (Her-2); EREG; ERK8; ESR1; ESR2; ETBR (Endothelin type B receptor); F3 (TF); FADD; FasL; FASN; FCER1A; FCER2; FCGR3A; FcRH1 (Fc receptor-like protein 1); FcRH2 (IFGP4, IRTA4, SPAP1A (SH2 domain containing phosphatase anchor protein 1a), SPAP1B, SPAP1C); FGF; FGF1 (αFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR; FGFR3; FIGF (VEGFD); FEL1 (EPSILON); FIL1 (ZETA); FLJ12584; FLJ25530; FLRTI (fibronectin); FLT1; FOS; FOSL1 (FRA-1); FY (DARC); GABRP (GABAa); GAGEB1; GAGEC1; GALNAC4S-6ST; GATA3; GDF5; GDNF-Ra1 (GDNF family receptor alpha 1; GFRA1; GDNFR; GDNFRA; RETL1; TRNR1; RET1L; GDNFR-alphal; GFR-ALPHA-1); GEDA; GFI1; GGT1; GM-CSF; GNASI; GNRHI; GPR2 (CCR10); GPR19 (G protein-coupled receptor 19; Mm.4787); GPR31; GPR44; GPR54 (KISS' receptor; KISS1R; GPR54; HOT7T175; AXOR12); GPR81 (FKSG80); GPR172A (G protein-coupled receptor 172A; GPCR41; FLJ11856; D15Ertd747e);GRCCIO (C10); GRP; GSN (Gelsolin); GSTP1; HAVCR2; HDAC4; HDACS; HDAC7A; HDAC9; HGF; HIF1A; HOP1; histamine and histamine receptors; HLA-A; HLA-DOB (Beta subunit of MHC class II molecule (Ia antigen); HLA-DRA; HM74; HMOXI ; HUMCYT2A; ICEBERG; ICOSL; 1D2; IFN-a; IFNA1; IFNA2; IFNA4; IFNA5; IFNA6; IFNA7; IFNB1; IFNgamma; DFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; ILIA; IL1B; ILIF10; IL1F5; IL1F6; IL1F7; IL1F8; IL1F9; IL1HY1; IL1R1; IL1R2; IL1RAP; IL1RAPL1; IL1RAPL2; IL1RL1; IL1RL2, ILIRN; IL2; IL20; IL20Roc; IL21 R; IL22; IL-22c; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); influenza A; influenza B; EL7; EL7R; EL8; IL8RA; DL8RB; IL8RB; DL9; DL9R; DLK; INHA; INHBA; INSL3; INSL4; IRAK1; IRTA2 (Immunoglobulin superfamily receptor translocation associated 2); ERAK2; ITGA1; ITGA2; ITGA3; ITGA6 (a6 integrin); ITGAV; ITGB3; ITGB4 (b4 integrin); α4β7 and αEβ7 integrin heterodimers; JAG1; JAK1; JAK3; JUN; K6HF; KAI1; KDR; KITLG; KLFS (GC Box BP); KLF6; KLKIO; KLK12; KLK13; KLK14; KLK15; KLK3; KLK4; KLKS; KLK6; KLK9; KRT1; KRT19 (Keratin 19); KRT2A; KHTHB6 (hair-specific type H keratin); LAMAS; LEP (leptin); LGRS (leucine-rich repeat-containing G protein-coupled receptor 5; GPR49, GPR67); Lingo-p75; Lingo-Troy; LPS; LTA (TNF-b); LTB; LTB4R (GPR16); LTB4R2; LTBR; LY64 (Lymphocyte antigen 64 (RP105), type I membrane protein of the leucine rich repeat (LRR) family); Ly6E (lymphocyte antigen 6 complex, locus E; Ly67,RIG-E,SCA-2,TSA-1); Ly6G6D (lymphocyte antigen 6 complex, locus G6D; Ly6-D, MEGT1); LY6K (lymphocyte antigen 6 complex, locus K; LY6K; HSJ001348; FLJ35226); MACMARCKS; MAG or OMgp; MAP2K7 (c-Jun); MDK; MDP; MIB1; midkine; MEF; MIP-2; MKI67; (Ki-67); MMP2; MMP9; MPF (MPF, MSLN, SMR, megakaryocyte potentiating factor, mesothelin); MS4A1; MSG783 (RNF124, hypothetical protein FLJ20315);MSMB; MT3 (metallothionectin-111); MTSS1; MUC1 (mucin); MYC; MY088; Napi3b (also known as NaPi2b) (NAPI-3B, NPTIIb, SLC34A2, solute carrier family 34 (sodium phosphate), member 2, type II sodium-dependent phosphate transporter 3b); NCA; NCK2; neurocan; NFKB1; NFKB2; NGFB (NGF); NGFR; NgR-Lingo; NgR- Nogo66 (Nogo); NgR-p75; NgR-Troy; NME1 (NM23A); NOXS; NPPB; NR0B1; NR0B2; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NTSE; NTN4; ODZI; OPRD1; OX40; P2RX7; P2X5 (Purinergic receptor P2X ligand-gated ion channel 5); PAP; PART1; PATE; PAWR; PCA3; PCNA; PD-Ll; PD-L2; PD-1; POGFA; POGFB; PECAM1; PF4 (CXCL4); PGF; PGR; phosphacan; PIAS2; PIK3CG; PLAU (uPA); PLG; PLXDC1; PMEL17 (silver homolog; SILV; D12S53E; PMEL17; SI; SIL); PPBP (CXCL7); PPID; PRI; PRKCQ; PRKDI; PRL; PROC; PROK2; PSAP; PSCA hlg (2700050C12Rik, C530008O16Rik, RIKEN cDNA 2700050C12, RIKEN cDNA 2700050C12 gene); PTAFR; PTEN; PTGS2 (COX-2); PTN; RAC2 (p21 Rac2); RARE; RET (ret proto-oncogene; MEN2A; HSCR1; MEN2B; MTC1; PTC; CDHF12; Hs.168114; RET51; RET-ELE1); RGSI; RGS13; RGS3; RNF110 (ZNF144); ROBO2; S100A2; SCGB1D2 (lipophilin B); SCGB2A1 (mammaglobin2); SCGB2A2 (mammaglobin 1); SCYEI (endothelial Monocyte-activating cytokine); SDF2; Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (™) and short cytoplasmic domain, (semaphorin) 5B); SERPINA1; SERPINA3; SERP1NB5 (maspin); SERPINE1(PAI-1); SERPDMF1; SHBG; SLA2; SLC2A2; SLC33A1; SLC43A1; SLIT2; SPPI; SPRR1B (Sprl); ST6GAL1; STABI; STAT6; STEAP (six transmembrane epithelial antigen of prostate); STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein); TB4R2; TBX21; TCPIO; TOGFI; TEK; TENB2 (putative transmembrane proteoglycan); TGFA; TGFBI; TGFB1II; TGFB2; TGFB3; TGFBI; TGFBRI; TGFBR2; TGFBR3; THIL; THBSI (thrombospondin-1); THBS2; THBS4; THPO; TIE (Tie-1); TMP3; tissue factor; TLR1; TLR2; TLR3; TLR4; TLRS; TLR6; TLR7; TLR8; TLR9; TLR10; TMEFF1 (transmembrane protein with EGF-like and two follistatin-like domains 1; Tomoregulin-1); TMEM46 (shisa homolog 2); TNF; TNF-α; TNFAEP2 (B94); TNFAIP3; TNFRSFIIA; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (AP03L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF5 (CD30 ligand); TNFSF9 (4-1 BB ligand); TOLLIP; Toll-like receptors; TOP2A (topoisomerase Ea); TP53; TPM1; TPM2; TRADD; TMEM118 (ring finger protein, transmembrane 2; RNFT2; FLJ14627); TRAF1; TRAF2; TRAF3; TRAF4; TRAFS; TRAF6; TREM1; TREM2; TrpM4 (BR22450, F1120041, TRPM4, TRPM4B, transient receptor potential cation channel, subfamily M, member 4); TRPC6; TSLP; TWEAK; Tyrosinase (TYR; OCAIA; OCA1A; tyrosinase; SHEP3);VEGF; VEGFB; VEGFC; versican; VHL C5; VLA-4; XCL1 (lymphotactin); XCL2 (SCM-lb); XCRI(GPRS/CCXCRI); YY1; and ZFPM2.

In certain embodiments, target molecules for antibodies (or bispecific antibodies) produced according to the methods provided herein include CD proteins such as CD3, CD4, CDS, CD16, CD19, CD20, CD21 (CR2 (Complement receptor 2) or C3DR (C3d/Epstein Barr virus receptor) or Hs.73792); CD33; CD34; CD64; CD72 (B-cell differentiation antigen CD72, Lyb-2); CD79b (CD79B, CD79β, IGb (immunoglobulin-associated beta), B29); CD200 members of the ErbB receptor family such as the EGF receptor, HER2, HER3, or HER4 receptor; cell adhesion molecules such as LFA-1, Macl, p150.95, VLA-4, ICAM-1, VCAM, alpha4/beta7 integrin, and alphav/beta3 integrin including either alpha or beta subunits thereof (e.g., anti-CD11a, anti-CD18, or anti-CD11b antibodies); growth factors such as VEGF-A, VEGF-C; tissue factor (TF); alpha interferon (alphaIFN); TNFalpha, an interleukin, such as IL-1 beta, IL-3, IL-4, IL-5, IL-6, IL-8, IL-9, IL-13, IL 17 AF, IL-1S, IL-13R alphal, IL13R alpha2, IL-4R, IL-5R, IL-9R, IgE; blood group antigens; flk2/flt3 receptor; obesity (OB) receptor; mpl receptor; CTLA-4; RANKL, RANK, RSV F protein, protein C etc.

In certain embodiments, the methods provided herein can be used to produce an antibody (or a multispecific antibody, such as a bispecific antibody) that specifically binds to complement protein C5 (e.g., an anti-C5 agonist antibody that specifically binds to human C5). In some embodiments, the anti-C5 antibody comprises 1, 2, 3, 4, 5, or 6 HVRs selected from (a) an HVR-H1 comprising the amino acid sequence of SSYYMA (SEQ ID NO:1); (b) HVR-H2 comprising the amino acid sequence of AIFTGSGAEYKAEWAKG (SEQ ID NO:26); (c) HVR-H3 comprising the amino acid sequence of DAGYDYPTHAMHY (SEQ ID NO: 27); (d) HVR-L1 comprising the amino acid sequence of RASQGISSSLA (SEQ ID NO: 28); (e) HVR-L2 comprising the amino acid sequence of GASETES (SEQ ID NO: 29); and (f) HVR-L3 comprising the amino acid sequence of QNTKVGSSYGNT (SEQ ID NO: 30). For example, in some embodiments, the anti-C5 antibody comprises a heavy chain variable domain (VH) sequence comprising one, two or three HVRs selected from: (a) an HVR-H1 comprising the amino acid sequence of (SSYYMA (SEQ ID NO: 1); (b) HVR-H2 comprising the amino acid sequence of AIFTGSGAEYKAEWAKG (SEQ ID NO: 26); (c) HVR-H3 comprising the amino acid sequence of DAGYDYPTHAMHY (SEQ ID NO: 27); and/or a light chain variable domain (VL) sequence comprising one, two or three HVRs selected from (d) HVR-L1 comprising the amino acid sequence of RASQGISSSLA (SEQ ID NO: 28); (e) HVR-L2 comprising the amino acid sequence of GASETES (SEQ ID NO: 29); and (f) HVR-L3 comprising the amino acid sequence of QNTKVGSSYGNT (SEQ ID NO: 30). The HVR-H1, HVR-H2, HVR-H3, HVR-L1, HVR-L2, and HVR-L3 sequences above are disclosed in US 2016/0176954 as SEQ ID NO: 117, SEQ ID NO: 118, SEQ ID NO: 121, SEQ ID NO: 122, SEQ ID NO: 123, and SEQ ID NO: 125, respectively. (See Tables 7 and 8 in US 2016/0176954.)

In certain embodiments, the anti-C5 antibody comprises the VH and VL sequences in
QVQLVESGGG LVQGRSLRL SCAASGFTVH SSYYMAWVRQ APGKGLEWVG AIFTGSGAEY KAEWAKGRVT ISKDTSKNQV VLTMTNMDPV DTATYYCASD AGYDYPTHAM HYWGQGTLVT vss (SEQ ID NO: 31)
and
DIQMTQSPSS LSASVGDRVT ITCRASQGIS SSLAWYQQKP GKAPKLLIYG ASETESGVPS RFSGSGSGTD FTLTISSLQP EDFATYYCQN TKVGSSYGNT FGGGTKVEIK (SEQEDNO:32), respectively, including post-translational modifications of those sequences. The VH and VL sequences above are disclosed in US 2016/0176954 as SEQ ID NO: 106 and SEQ ID NO: 111, respectively. (See Tables 7 and 8 in US 2016/0176954.) In some embodiments, the anti-C5 antibody is 305L015 (see US 2016/0176954).

In certain embodiments, the methods provided herein can be used to produce an antibody (or a multispecific antibody, such as a bispecific antibody) that specifically binds to OX40 (e.g., an anti-OX40 agonist antibody that specifically binds to human OX40). In some embodiments, the anti-OX40 antibody comprises 1, 2, 3, 4, 5, or 6 HVRs selected from (a) an HVR-H1 comprising the amino acid sequence of DSYMS (SEQ ID NO: 2); (b) HVR-H2 comprising the amino acid sequence of DMYPDNGDSSYNQKFRE (SEQ ID NO: 3); (c) HVR-H3 comprising the amino acid sequence of APRWYFSV (SEQ ID NO: 4); (d) HVR-L1 comprising the amino acid sequence of RASQDISNYLN (SEQ ID NO: 5); (e) HVR-L2 comprising the amino acid sequence of YTSRLRS (SEQ ID NO: 6); and (f) HVR-L3 comprising the amino acid sequence of QQGHTLPPT (SEQ ID NO: 7). For example, in some embodiments, the anti-OX40 antibody comprises a heavy chain variable domain (VH) sequence comprising one, two or three HVRs selected from: (a) an HVR-H1 comprising the amino acid sequence of DSYMS (SEQ ID NO: 2); (b) HVR-H2 comprising the amino acid sequence of DMYPDNGDSSYNQKFRE (SEQ ID NO: 3); and (c) HVR-H3 comprising the amino acid sequence of APRWYFSV (SEQ ID NO: 4) and/or a light chain variable domain (VL) sequence comprising one, two or three HVRs selected from (a) HVR-L1 comprising the amino acid sequence of RASQDISNYLN (SEQ ID NO: 5); (b) HVR-L2 comprising the amino acid sequence of YTSRLRS (SEQ ID NO: 6); and (c) HVR-L3 comprising the amino acid sequence of QQGHTLPPT (SEQ ID NO: 7). In certain embodiments, the anti-OX40antibody comprises the VH and VL sequences in
EVQLVQSGAE VKKPGASVKV SCKASGYTFT DSYMSWVRQA PGQGLEWIGD MYPDNGDSSY NQKFRERVTI TRDTSTSTAY LELSSLRSED TAVYYCVLAP RWYFSVWGQG TLVTVSS (SEQ ID NO: 8)
and
DIQMTQSPSS LSASVGDRVT ITCRASQDIS NYLNWYQQKP GKAPKLLIYY TSRLRSGVPS RFSGSGSGTD FTLTISSLQP EDFATYYCQQ GHTLPPTFGQ GTKVEIK (SEQ ID NO: 9), respectively, including post-translational modifications of those sequences.

In some embodiments, the anti-OX40 antibody comprises 1, 2, 3, 4, 5, or 6 HVRs selected from (a) an HVR-H1 comprising the amino acid sequence of NYLIE (SEQ ID NO: 10); (b) HVR-H2 comprising the amino acid sequence of VINPGSGDTYYSEKFKG (SEQ ID NO: 11; (c) HVR-H3 comprising the amino acid sequence of DRLDY (SEQ ID NO: 12); (d) HVR-L1 comprising the amino acid sequence of HASQDISSYIV (SEQ ID NO: 13); (e) HVR-L2 comprising the amino acid sequence of HGTNLED (SEQ ID NO: 14); and (f) HVR-L3 comprising the amino acid sequence of VHYAQFPYT (SEQ ID NO: 15). For example, in some embodiments, the anti-OX40 antibody comprises a heavy chain variable domain (VH) sequence comprising one, two or three HVRs selected from: (a) an HVR-H1 comprising the amino acid sequence of NYLIE (SEQ ID NO: 10); (b) HVR-H2 comprising the amino acid sequence of VINPGSGDTYYSEKFKG (SEQ ID NO: 11); and (c) HVR-H3 comprising the amino acid sequence of DRLDY (SEQ ID NO: 12) and/or a light chain variable domain (VL) sequence comprising one, two or three HVRs selected from (a) HVR-L1 comprising the amino acid sequence of HASQDISSYIV (SEQ ID NO: 13); (b) HVR-L2 comprising the amino acid sequence of HGTNLED (SEQ ID NO: 14); and (c) HVR-L3 comprising the amino acid sequence of VHYAQFPYT (SEQ ID NO: 15). In certain embodiments, the anti-OX40 antibody comprises the VH and VL sequences in
EVQLVQSGAE VKKPGASVKV SCKASGYAFT NYLIEWVRQA PGQGLEWIGV INPGSGDTYY SEKFKGRVTI TRDTSTSTAY LELSSLRSED TAVYYCARDR LDYWGQGTLV TVSS (SEC ID NO: 16)
and
DIQMTQSPSS LSASVGDRVT ITCHASQDIS SYIVWYQQKP GKAPKLLIYH GTNLEDGVPS RFSGSGSGTD FTLTISSLQP EDFATYYCVH YAQFPYTFGQ GTKVEIK (SE( )ID NO: 17), respectively, including post-translational modifications of those sequences.

Further details regarding anti-OX40 antibodies are provided in WO 2015/153513, which is incorporated herein by reference in its entirety.

In certain embodiments, the methods provided herein can be used to produce an antibody (or a multispecific antibody, such as a bispecific antibody) that specifically binds to influenza virus B hemagglutinin, i.e., “fluB” (e.g., an antibody that binds hemagglutinin from the Yamagata lineage of influenza B viruses, binds hemagglutinin from the Victoria lineage of influenza B viruses, binds hemagglutinin from ancestral lineages of influenza B virus, or binds hemagglutinin from the Yamagata lineage, the Victoria lineage, and ancestral lineages of influenza B virus, in vitro and/or in vivo). Further details regarding anti-FluB antibodies are described in WO 2015/148806, which is incorporated herein by reference in its entirety.

In certain embodiments, an antibody (or bispecific antibody) produced according to a method provided herein binds low density lipoprotein receptor-related protein (LRP)-1 or LRP-8 or transferrin receptor, and at least one target selected from the group consisting of beta-secretase (BACE1 or BACE2), alpha-secretase, gamma-secretase, tau-secretase, amyloid precursor protein (APP), death receptor 6 (DR6), amyloid beta peptide, alpha-synuclein, Parkin, Huntingtin, p75 NTR, CD40 and caspase-6.

In certain embodiments, the antibody produced according to a method provided herein is a human IgG2 antibody against CD40. In certain embodiments, the anti-CD40 antibody is RG7876.

In certain embodiments, the polypeptide produced according to a method provided herein is a targeted immunocytokine. In certain embodiments, the targeted immunocytokine is a CEA-IL2v immuocytokine. In certain embodiments, the CEA-IL2v immuocytokine is RG7813. In certain embodiments, the targeted immunocytokine is a FAP-IL2v immuocytokine. In certain embodiments, the FAP-IL2v immunocytokine is RG7461.

In certain embodiments, a multispecific antibody (such as a bispecific antibody) produced according to a method provided herein binds CEA and at least one additional target molecule. In certain embodiments, a multispecific antibody (such as a bispecific antibody) produced according to a method provided herein binds a tumor targeted cytokine and at least one additional target molecule. In certain embodiments, a multispecific antibody (such as a bispecific antibody) produced according to a method provided herein is fused to IL2v (i.e., an interleukin 2 variant) and binds an IL1-based immunocytokine and at least one additional target molecule. In certain embodiments, a multispecific antibody (such as a bispecific antibody) produced according to a method provided herein is a T-cell bispecific antibody (i.e., a bispecific T-cell engager or BiTE).

In certain embodiments, a multispecific antibody (such as a bispecific antibody) produced according to a method provided herein binds to at least two target molecules selected from: IL-1 alpha and IL- 1 beta, IL-12 and IL-1S; IL-13 and IL-9; IL-13 and IL-4; IL-13 and IL-5; IL-5 and IL-4; IL-13 and IL-lbeta; IL-13 and IL- 25; IL-13 and TARC; IL-13 and MDC; IL-13 and MEF; IL-13 and TGF-˜; IL-13 and LHR agonist; IL-12 and TWEAK, IL-13 and CL25; IL-13 and SPRR2a; IL-13 and SPRR2b; IL-13 and ADAMS, IL-13 and PED2, IL17A and IL17F, CEA and CD3, CD3 and CD19, CD138 and CD20; CD138 and CD40; CD19 and CD20; CD20 and CD3; CD3S and CD13S; CD3S and CD20; CD3S and CD40; CD40 and CD20; CD-S and IL-6; CD20 and BR3, TNF alpha and TGF-beta, TNF alpha and IL-1 beta; TNF alpha and IL-2, TNF alpha and IL-3, TNF alpha and IL-4, TNF alpha and IL-5, TNF alpha and IL6, TNF alpha and IL8, TNF alpha and IL-9, TNF alpha and IL-10, TNF alpha and IL-11, TNF alpha and IL-12, TNF alpha and IL-13, TNF alpha and IL-14, TNF alpha and IL-15, TNF alpha and IL-16, TNF alpha and IL-17, TNF alpha and IL-18, TNF alpha and IL-19, TNF alpha and IL-20, TNF alpha and IL-23, TNF alpha and IFN alpha, TNF alpha and CD4, TNF alpha and VEGF, TNF alpha and MIF, TNF alpha and ICAM-1, TNF alpha and PGE4, TNF alpha and PEG2, TNF alpha and RANK ligand, TNF alpha and Te38, TNF alpha and BAFF,TNF alpha and CD22, TNF alpha and CTLA-4, TNF alpha and GP130, TNF a and IL-12p40, VEGF and Angiopoietin, VEGF and HER2, VEGF-A and HER2, VEGF-A and PDGF, HER1 and HER2, VEGFA and ANG2,VEGF-A and VEGF-C, VEGF-C and VEGF-D, HER2 and DRS,VEGF and IL-8, VEGF and MET, VEGFR and MET receptor, EGFR and MET, VEGFR and EGFR, HER2 and CD64, HER2 and CD3, HER2 and CD16, HER2 and HER3; EGFR (HER1) and HER2, EGFR and HER3, EGFR and HER4, IL-14 and IL-13, IL-13 and CD40L, IL4 and CD40L, TNFR1 and IL-1 R, TNFR1 and IL-6R and TNFR1 and IL-18R, EpCAM and CD3, MAPG and CD28, EGFR and CD64, CSPGs and RGM A; CTLA-4 and BTN02; IGF1 and IGF2; IGF1/2 and Erb2B; MAG and RGM A; NgR and RGM A; NogoA and RGM A; OMGp and RGM A; POL-1 and CTLA-4; and RGM A and RGM B.

In certain embodiments, the multispecific antibody (such as a bispecific antibody) is an anti-CEA/anti-CD3 bispecific antibody. In certain embodiments, the anti-CEA/anti-CD3 bispecific antibody is RG7802. In certain embodiments, the anti-CEA/anti-CD3 bispecific antibody comprises the amino acid sequences set forth in SEQ ID NOs 18-21 are provided below:

(SEQ ID NO: 18)
DIQMTQSPSS LSASVGDRVT ITCKASAAVG TYVAWYQQKP
GKAPKLLIYS ASYRKRGVPS RFSGSGSGTD FTLTISSLQP
EDFATYYCHQ YYTYPLFTFG QGTKLEIKRT VAAPSVFIFP
PSDEQLKSGT ASVVCLLNNF YPREANVQWN VDNALQSGNS
QESVTEQDSK DSTYSLSSTL TLSKADYEKH KVYACEVTHQ
GLSSPVTKSF NRGEC
(SEQ ID NO: 19)
QAVVTQEPSL TVSPGGTVTL TCGSSTGAVT TSNYANWVQE
KPGQAFRGLI GGTNNRAPGT PARFSGSLLG GKAALTLSGA
QPEDEAEYYC ALWYSNLWVF GGGTKLTVLS SASTKGPSVF
PLAPSSKSTS GGTAALGCLV KDYFPEPVTV SWNSGALTSG
VHTFPAVLQS SGLYSLSSVV TVPSSSLGTQ TYICNVNHKP
SNTKVDKKVE PKSC
(SEQ ID NO: 20)
QVQLVQSGAE VKKPGASVKV SCKASGYTFT EFGMNWVRQA
PGQGLEWMGW INTKTGEATY VEEFKGRVTF TTDTSTSTAY
MELRSLRSDD TAVYYCARWD FAYYVEAMDY WGQGTTVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKKVE PKSCDGGGGS GGGGSEVQLL
ESGGGLVQPG GSLRLSCAAS GFTFSTYAMN WVRQAPGKGL
EWVSRIRSKY NNYATYYADS VKGRFTISRD DSKNTLYLQM
NSLRAEDTAV YYCVRHGNFG NSYVSWFAYW GQGTLVTVSS
ASVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGECDKT HTCPPCPAPE
AAGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALGAPIE KTISKLKGQP REPQVYTLPP
CRDELTKNQV SLWCLVKGFY PSDIAVEWES NGQPENNYKT
TPPVLDSDGS FFLYSKLTVD KSRWQQGNVF SCSVMHEALH
NHYTQKSLSL SPGK
(SEQ ID NO: 21)
QVQLVQSGAE VKKPGASVKV SCKASGYTFT EFGMNWVRQA
PGQGLEWMGW INTKTGEATY VEEFKGRVTF TTDTSTSTAY
MELRSLRSDD TAVYYCARWD FAYYVEAMDY WGQGTTVTVS
SASTKGPSVF PLAPSSKSTS GGTAALGCLV KDYFPEPVTV
SWNSGAITSG VHTFPAVLQS SGLYSLSSVV TVPSSSLGTQ
TYICNVNHKP SNTKVDKKVE PKSCDKTHTC PPCPAPEAAG
GPSVELEPPK PHDTLMISRT PEVTCVVVDV SHEDPEVKFN
WYVDGVEVHN AKTKPREEQY NSTYRVVSVL TVLHQDWLNG
KEYKCKVSNK ALGAPIEKTI SKAKGQPREP QVCTLPPSRD
ELTKNQVSLS CAVNGFYPSD IAVEWESNGQ PENNYKTTPP
VLDSDGSFFL VSKLTVDKSR WQQGNVESCS VMHEALHNHY
TQKSLSLSPG K

Further details regarding anti-CEA/anti-CD3 bispecific antibodies are provided in WO 2014/121712, which is incorporated herein by reference in its entirety.

In certain embodiments, the multispecific antibody (such as a bispecific antibody) is an anti-VEGF/anti-angiopoietin bispecific antibody. In certain embodiments, the anti-VEGF/anti-angiopoietin bispecific antibody bispecific antibody is a Crossmab. In certain embodiments, the anti-VEGF/anti-angiopoietin bispecific antibody is RG7716. In certain embodiments, the anti-CEA/anti-CD3 bispecific antibody comprises the amino acid sequences set forth in SEQ ID NOs 22-25 are provided below:

(SEQ ID NO: 22)
EVQLVESGGG LVQPGGSLRL SCAASGYDFT HYGMNWVRQA
PGKGLEWVGW INTYTGEPTY AADFKRRFTF SLDTSKSTAY
LQMNSLRAED TAVYYCAKYP YYYGTSHWYF DVWGQGTLVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKK VEPKSCDKTH TCPPCPAPEA
AGGPSVFLFP PKPKDTLMAS RTPEVTCVVV DVSHEDPEVK
FNWYVDGVEV HNAKTKPREE QYNSTYRVVS VLTVLAQDWL
NGKEYKCKVS NKALGAPIEK TISKAKGQPR EPQVYTLPPC
RDELTKNQVS LWCLVKGFYP SDIAVEWESN GQPENNYKTT
PPVLDSDGSF FLYSKLTVDK SRWQQGNVFS CSVMHEALHN
AYTQKSLSLS PGK
(SEQ ID NO: 23)
QVQLVQSGAE VKKPGASVKV SCKASGYTFT GYYMHWVRQA
PGQGLEWMGW INPNSGGTNY AQKFQGRVTM TRDTSISTAY
MELSRLRSDD TAVYYCARSP NPYYYDSSGY YYPGAFDIWG
QGTMVTVSSA SVAAPSVFIF PPSDEQLKSG TASVVCLLNN
FYPREAKVQW KVDNALQSGN SQESVTEQDS KDSTYSLSST
LTLSKADYEK HKVYACEVTH QGLSSPVTKS FNRGECDKTH
TCPPCPAPEA AGGPSVFLFP PKPKDTLMAS RTPEVTCVVV
DVSHEDPEVK FNWYVDGVEV HNAKTKPREE QYNSTYRVVS
VLTVLAQDWL NGKEYKCKVS NKALGAPIEK TISKAKGQPR
EPQVCTLPPS RDELTKNQVS LSCAVKGFYP SDIAVEWESN
GQPENNYKTT PPVLDSDGSF FLVSKLTVDK SRWQQGNVES
CSVMHEALHN AYTQKSLSLS PGK
(SEQ ID NO: 24)
DIQLTQSPSS LSASVGDRVT ITCSASQDIS NYLNWYQQKP
GKAPNVLIYF TSSLHSGVPS RFSGSGSGTD FTLTISSLQP
EDFATYYCQQ YSTVPWTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC
(SEQ ID NO: 25)
SYVLTQPPSV SVAPGQTARI TCGGNNIGSK SVHWYQQKPG
QAPVLVVYDD SDRPSGIPER FSGSNSGNTA TLTISRVEAG
DEADYYCQVW DSSSDHWVFG GGTKLTVLSS ASTKGPSVFP
LAPSSKSTSG GTAALGCLVK DYFPEPVTVS WNSGALTSGV
HTFPAVLQSS GLYSLSSVVT VPSSSLGTQT YICNVNHKPS
NTKVDKKVEP KSC

In certain embodiments, the multispecific antibody (such as a bispecific antibody) is an anti-Ang2/anti-VEGF bispecific antibody. In certain embodiments, the anti-Ang2/anti-VEGF bispecific antibody is RG7221. In certain embodiments, the anti-Ang2/anti-VEGF bispecific antibody is CAS Number 1448221-05-3.

Soluble antigens or fragments thereof, optionally conjugated to other molecules, can be used as immunogens for generating antibodies. For transmembrane molecules, such as receptors, fragments of these (e.g., the extracellular domain of a receptor) can be used as the immunogen. Alternatively, cells expressing the transmembrane molecule can be used as the immunogen. Such cells can be derived from a natural source (e.g., cancer cell lines) or may be cells which have been transformed by recombinant techniques to express the transmembrane molecule. Other antigens and forms thereof useful for preparing antibodies will be apparent to those in the art.

In certain embodiments, the polypeptide (e.g., antibodies) produced herein can be further conjugated to a chemical molecule such as a dye or cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). An immunoconjugate comprising an antibody or bispecific antibody produced using a method described herein may contain the cytotoxic agent conjugated to a constant region of only one of the heavy chains or only one of the light chains.

### C. Pharmaceutical Compositions and Formulations

The polypeptides (e.g., antibodies or bispecific antibodies) produced according to the methods provided herein can be formulated with suitable carriers or excipients so that they are suitable for administration. Suitable formulations of the polypeptides (e.g., antibodies or bispecific antibodies) produced according to the methods provided herein are obtained by mixing polypeptides (e.g., antibodies or bispecific antibodies) having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Exemplary antibody formulations are described in W098/56418, expressly incorporated herein by reference. Lyophilized formulations adapted for subcutaneous administration are described in W097 /04801. Such lyophilized formulations may be reconstituted with a suitable diluent to a high protein concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent. Such molecules are suitably present in combination in amounts that are effective for the purpose intended. The effective amount of such other agents depends on the amount of polypeptide (e.g., antibody or bispecific antibody) present in the formulation, the type of disease or disorder or treatment, and other factors discussed above. These are generally used in the same dosages and with administration routes as described herein or about from 1 to 99% of the heretofore employed dosages. The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Sustained-release preparations may be prepared. Suitable examples of sustained release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and. ethyl-L-glutamate, non-degradable ethylene-vinyl, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

Optionally, but preferably, the formulation contains a pharmaceutically acceptable salt, preferably sodium chloride, and preferably at about physiological concentrations. Optionally, the formulations can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations can include a pharmaceutically acceptable surfactant at a concentration of 0.005 to 0.02%.

Sustained-release preparations may be prepared. Suitable examples of sustained-release preparations include semipermeable matrices of solid hydrophobic polymers containing a polypeptide (e.g., antibody or bispecific antibody) produced according to a method provided herein, which matrices are in the form of shaped articles, e.g., films, or microcapsule. Examples of sustained-release matrices include polyesters, hydrogels (for example, poly(2-hydroxyethylmethacrylate), or poly(vinylalcohol)), polylactides (U.S. Pat. No. 3,773,919), copolymers of L-glutamic acid and ethyi-L-glutamate, non-degradable ethylene- vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated polypeptide(s) (e.g., antibodies or bispecific antibodies) produced according to the methods provided herein remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

The polypeptides (e.g., antibodies or bispecific antibodies) produced according to the methods provided herein are administered to a human subject, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Local administration may be particularly desired if extensive side effects or toxicity is associated with antagonism to the target molecule recognized by the proteins. An ex vivo strategy can also be used for therapeutic applications. Ex vivo strategies involve transfecting or transducing cells obtained from the subject with a polynucleotide encoding a protein provided herein. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells.

In one example, the polypeptide (e.g., antibody or bispecific antibody) produced according to a method provided herein is administered locally, e.g., by direct injections, when the disorder or location of the tumor permits, and the injections can be repeated periodically. The polypeptide (e.g., antibody or bispecific antibody) can also be delivered systemically to the subject or directly to the tumor cells, e.g., to a tumor or a tumor bed following surgical excision of the tumor, in order to prevent or reduce local recurrence or metastasis.

### D. Articles of Manufacture and Kits

Also provided are articles of manufacture containing one or more polypeptides (e.g., antibodies or bispecific antibodies) produced according to a method provided herein, and materials useful for the treatment or diagnosis of a disorder (for example, an autoimmune disease or cancer). In certain embodiments, the article of manufacture comprises a container and a label or package insert on or associated with the container. Suitable containers include, for example, bottles, vials, syringes, etc. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). At least one active agent in the composition is a polypeptide (e.g., antibody or bispecific antibody) produced according to a method provided herein. The label or package insert indicates that the composition is used for treating the particular condition. The label or package insert will further comprise instructions for administering the composition comprising a polypeptide (e.g., antibody or bispecific antibody) produced according to a method provided herein to the subject. Articles of manufacture and kits comprising combinatorial therapies described herein are also contemplated.

“Package insert” refers to instructions customarily included in commercial packages of therapeutic products that contain information about the indications, usage, dosage, administration, contra indications and/or warnings concerning the use of such therapeutic products. In certain embodiments, the package insert indicates that the composition is used for treating breast cancer, colorectal cancer, lung cancer, renal cell carcinoma, glioma, or ovarian cancer.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials considered from a commercial and user standpoint, including other buffers, diluents, filters, needles, and syringes.

Kits are also provided that are useful for various purposes, e.g., for purification or immunoprecipitation of two or more target antigens from cells. For isolation and purification of two or more target antigens, the kit can contain polypeptide (e.g., antibody or bispecific antibody) produced according to a method provided herein coupled to beads (e.g., sepharose beads). Kits can be provided which contain a polypeptide (e.g., an antibody or bispecific antibody) produced according to a method provided herein for detection and quantitation of the antigen in vitro, e.g., in an ELISA or a Western blot. As with the article of manufacture, the kit comprises a container and a label or package insert on or associated with the container. The container holds a composition comprising at least one polypeptide (e.g., antibody or bispecific antibody) produced according to a method provided herein. Additional containers may be included that contain, e.g., diluents and buffers or control antibodies. The label or package insert may provide a description of the composition as well as instructions for the intended in vitro or diagnostic use.

### EXAMPLES

The present disclosure will be more fully understood by reference to the following examples. They should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Effects of Cysteine, Cystine, Iron (Fe), and B Vitamins on Trisulfide Formation in a Recombinant Polypeptide in a Cell-Free System

A first series of experiments was conducted in a cell-free system to identify the components in a cell culture medium that contribute to trisulfide formation in an extracellularly secreted recombinant polypeptide. In particular, the experiments investigated the effect of cysteine, cystine, trace elements such as iron, and B vitamins on trisulfide bond levels in such polypeptide.

### A. Non-Cellular Effect of Cysteine, Cystine and Iron

Briefly, anti-FluB, an exemplary polypeptide containing 11% trisulfide, was incubated in Medium 1 that had been supplemented with (a) 6 mM L-cysteine (Cys), (b) 3 mM cystine (Cys-Cys), (c) 6 mM L-cysteine (Cys) and 35 μM Fe (iron), or (d) 3 mM cystine (Cys-Cys) and 35 μM Fe (iron). The incubations, supplemented as described above, were repeated in Medium 2, i.e., a medium that has a different composition than Medium 1. Further details regarding anti-FluB are described in WO 2015/148806, which is incorporated herein by reference in its entirety.

Medium 1 and Medium 2 differ in the number, types, and concentrations of nutrients and components they contain. Specifically, the concentrations of vitamin B2 and vitamin B6 in Medium 1 are different from the concentrations of vitamin B2 and vitamin B6 in Medium 2.

The final concentration of anti-FluB in the media was 1.5 g/L. The incubations were performed in an incubator with a temperature set point of 37° C., CO₂ set point of 5%. The incubation mixtures were held in capped tube spin bioreactors and were shaken at 225 rpm. Half the replicates were held in tube spins with vented caps and the other half of the replicates were held in tube spins with non-vented caps. The temperature, the CO₂, and the agitation of the shaken tube spin reactors were within relevant ranges for concentration of antibody in CHO antibody production cultures and temperatures used for CHO (or other mammalian) cell culture.

Samples from each of the eight incubations were taken at 0 h, 6 h, 24 h, and 72 h, and % trisulfide in anti-FluB at each time point was determined via hydrophobic interaction liquid chromatography combined with charged aerosol detection (HILIC-CAD), according to the method described in Zhang et al. (2010) Journal of Chromatography A. 1217, 5776-5784 and Cornell et al. (in press).

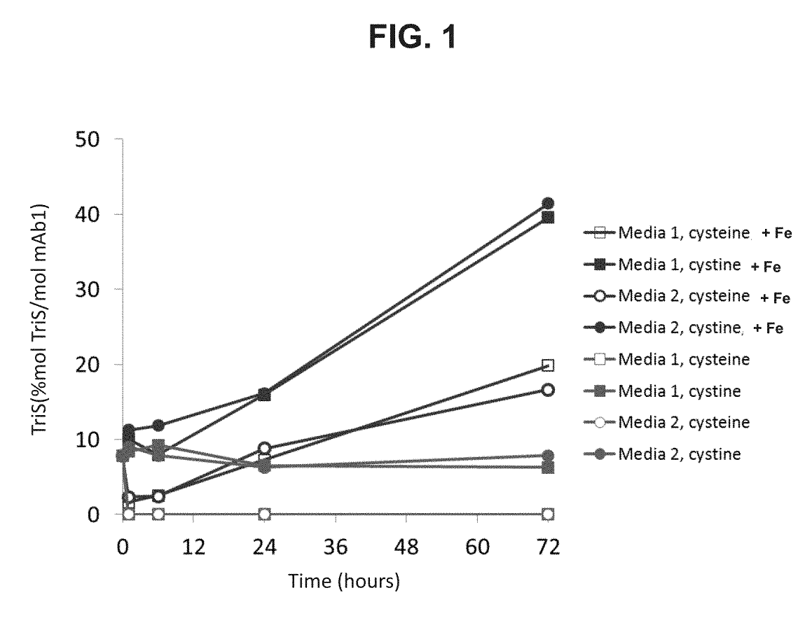

As shown in FIG. 1, incubation of anti-FluB in Medium 1+Cys or Medium 2+Cys rapidly decreased the trisulfide bond level from 11% to nearly 0%, and such effect was sustained for 72 hours. Trisulfide bond levels in anti-FluB were not significantly affected over the course of a 72 hour incubation in Medium 1+Cys-Cys or Medium 2+Cys-Cys. Trisulfide bond levels rapidly decreased when anti-FluB was incubated in Medium 1+Cys+Fe or Medium 2+Cys+Fe, and such effect was sustained for about 6 hours. After 6 hours, however, trisulfide bond levels in anti-FluB increased to about 15%, i.e., slightly higher than the initial trisulfide bond level. This observation is consistent with the amount of time required for Cys to convert to Cys-Cys in a cell-free system with Fe present, at which point the composition acts as one that contains Cys-Cys and Fe (see below). Incubation of anti-FluB in Medium 1+Cys-Cys+Fe or Medium 2+Cys-Cys+Fe significantly increased trisulfide bond levels to about 40% over the course of the 72 hour incubation. Similar results were observed with anti-FluB containing 45% trisulfide (data not shown). Gas exchange had no effect on trisulfide formation (data not shown).

Collectively, the results in FIG. 1 demonstrate that: 1) trisulfide bond levels are reduced when Cys is present, but that trisulfide bond levels can increase if Cys is allowed to convert to Cys-Cys; 2) Fe is required for trisulfide formation in extracellular antibody pools, and that trisulfide formation increases significantly in the presence of both Fe and cystine (Cys-Cys); and 3) the effect of Fe and cystine (Cys-Cys) on trisulfide formation does not appear to be dependent on the cell culture medium given the similarity in results observed in both Medium 1 and Medium 2.

The results shown in FIG. 1 also demonstrate that polysulfides such as cystine (Cys-Cys) may act as a sulfur pool for sulfur transfer to generate trisulfide bonds in antibodies. H₂S was detectable in the headspace when anti-FluB was incubated in Medium 1+Cys without Fe or in Medium 1+Cys+Fe (data not shown). Higher levels of H₂S were detected when anti-FluB was incubated in Medium 1+Cys+Fe (data not shown). H₂S(g) was not detectable in the headspace when anti-FluB was incubated in Medium 1+Cys-Cys+Fe or in Medium 1+Cys-Cys without Fe (data not shown). Without intending to be bound by one particular theory, such results suggest that the presence of Cys or Cys+Fe did not lead to the formation of H₂S (g) and therefore contributed to trisulfide formation even when H₂S (g) levels in the headspace were undetectable.

### B. Non-cellular Effect of Iron and B Vitamins

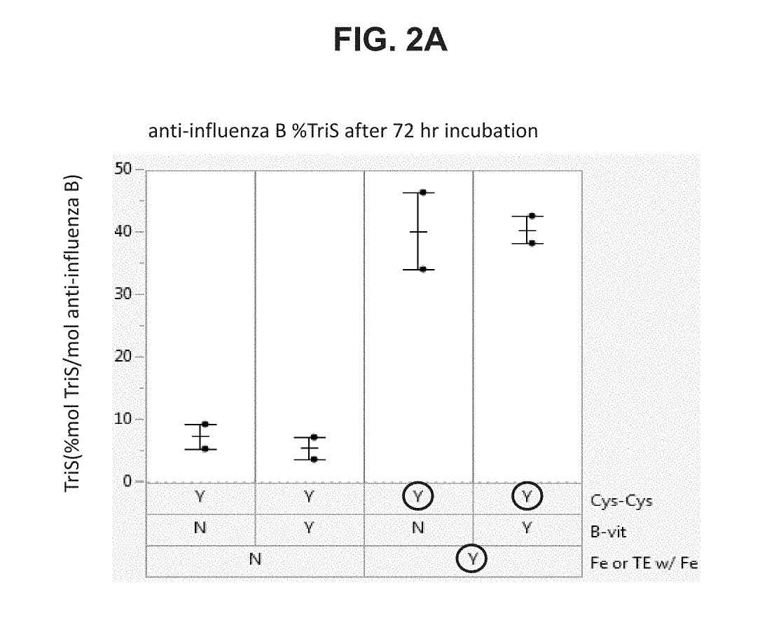

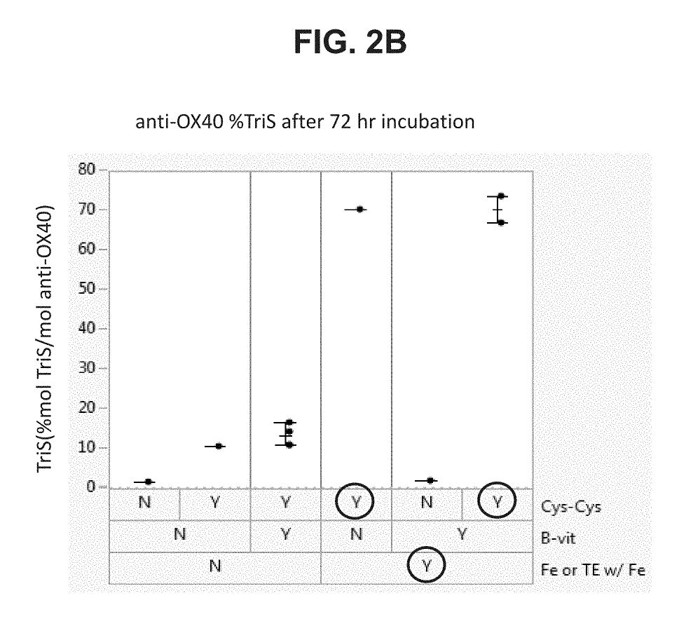

In a further set of experiments, anti-FluB was incubated for 72 hours in Medium 1 that was supplemented with one or more of the following components: (a) 3 mM cystine (Cys-Cys), (b) 35 μM Fe, and (c) B vitamins (1.84 μM riboflavin (vitamin B2), 24.9 μM pyridoxine (vitamin B6), 22.5 μM folic acid (vitamin B9), and 2.25 μM cyanocobalamin (vitamin B12)). As shown in FIG. 2A, trisulfide bond levels were not significantly affected when anti-FluB was incubated Medium 1 supplemented with Cys-Cys or with Cys-Cys+B vitamins. Trisulfide bond levels increased significantly, i.e. , from 11% to about 40%, when anti-FluB was incubated in Medium 1 supplemented with Fe +Cys-Cys or with Fe+Cys-Cys+B vitamins, although the trisulfide bond levels were approximately the same in the presence or absence of the B-vitamins when in a cell-free system.

The experiments described above were repeated using an anti-OX40 antibody, i.e., an exemplary polypeptide containing 1% trisulfide. The anti-OX40 antibody used in the present Examples comprises a heavy chain variable domain set forth in SEQ ID NO: 8 and a light chain variable domain set forth in SEQ ID NO: 9. SEQ ID NOs: 8 and 9 are provided below. Further details regarding anti-OX40 antibodies are provided in WO 2015/153513, which is incorporated herein by reference in its entirety.

SEQ ID NO: 8:
EVQLVQSGAE VKKPGASVKV SCKASGYTFT DSYMSWVRQA
PGQGLEWIGD MYPDNGDSSY NQKFRERVTI TRDTSTSTAY
LELSSLRSED TAVYYCVLAP RWYFSVWGQG TLVTVSS
SEQ ID NO: 9:
DIQMTQSPSS LSASVGDRVT ITCRASQDIS NYLNWYQQKP
GKAPKLLIYY TSRLRSGVPS RFSGSGSGTD FTLTISSLQP
EDFATYYCQQ GHTLPPTFGQ GTKVEIK

Incubation of anti-OX40 Ab in Medium 1 lacking Cys-Cys, Fe, and B vitamins had no effect on trisulfide bond levels. See FIG. 2B. Trisulfide bond levels also remained unchanged when anti-OX40 Ab was incubated in Medium 1 containing Fe +B vitamins. Trisulfide bond levels increased from 1% to about 10-15% when anti-OX40 Ab was incubated in Medium 1 supplemented with Cys-Cys or with both Cys-Cys and B vitamins. Trisulfide bond levels increased significantly, i.e., from 1% to about 75%, when anti-OX40 Ab was incubated in Medium 1 supplemented with Fe +Cys-Cys or with Fe +Cys-Cys+B vitamins. Again, the presence of B vitamins did not significantly impact trisulfide bond levels.

Taken together, the results of FIGS. 1 and 2A and 2B demonstrate that: 1) the presence of Fe and cystine (Cys-Cys) in the medium contributes to the formation of trisulfide bonds in polypeptides in a cell-free system, and 2) B vitamins (B2, B6, B9, and B12) do not significantly impact trisulfide bond levels when the antibody is incubated in a cell-free system (in contrast to the effect observed in a cell-culture system as noted below).

### Example 2

### Cell Culture Medium Components Affecting Trisulfide Formation in a Polypeptide Produced by a Mammalian Cell

Another series of experiments was conducted to assess the effects of cysteine (Cys), cystine (Cys-Cys), iron and B vitamins on trisulfide bond levels, but this time in a cell culture process rather than in a cell-free process to address what effect these compounds have in a cell culture environment.

### A. Effect of Cysteine and Cystine in Cell Culture

One set of cell culture experiments were performed to assess the effects of added cysteine (Cys) or added cystine (Cys-Cys) on trisulfide formation during culture. Briefly, CHO cells producing anti-OX40 Ab were cultured in 2 liter bioreactors over a 14 day run according to one of the four protocols shown in Table 1 below:

TABLE 1

| | | | | Theoretical |
|---|---|---|---|---|
| | | | | Calculation of Total |
| | Cysteine or Cys-Cys Concentrations | | | Cys or Cys-Cys |
| | | Addition to | | Added to Culture |
| | Basal Medium‡ | Culture from | Batch Feed | from Stock |
| | (1 liter) | Stock Solution | Strategy | Solution* |
| Protocol 1 | 3 mM Cys | 10 mL of 450 mM | 200 mL Batch Feed | 3.72 mM Cys |
| (Cys day 3) | | Cys on | Medium added on | |
| | | Day 3 | Day 3 | |
| | 0 mM Cys-Cys | 0 mM Cys-Cys | | |
| Protocol 2 | 0 mM Cys | 0 mM Cys | 200 mL Batch Feed | 1.86 mM Cys-Cys |
| (Cys-Cys day 3) | 1.5 mM Cys-Cys | 10 mL of 225 mM | Medium added on | |
| | | Cys-Cys on | Day 3 | |
| | | Day 3 | | |
| Protocol 3 | 3 mM Cys | 5 mL of 450 mM | 100 mL Batch Feed | 5.13 mM Cys |
| (Cys day 3, 6, 9) | | Cys on Day 3, 6, | Medium added on | |
| | | and 9 | Days 3, 6, and 9 | |
| | 0 mM Cys-Cys | 0 mM Cys-Cys | | |
| Protocol 4 | 0 mM Cys | 0 mM Cys | 100 mL Batch Feed | 2.565 mM Cys |
| (Cys-Cys day 3, | 1.5 mM Cys-Cys | 5 mL of 225 mM | Medium added on | |
| 6, 9) | | Cys-Cys on Day | Days 3, 6, and 9 | |
| | | 3, 6, and 9 | | |

‡at the start of production culture
*cellular consumption and generation not taken into consideration during calculation.

To initiate the growth phase of production cell cultures, CHO cells were inoculated at approximately 1.0×10⁶ cells/mL in 2-L stirred bioreactors (Applikon, Foster City, CA) containing 1L of basal medium. The cells were cultured in fed-batch mode with batch feed medium additions of either 100 mL per liter of cell culture fluid at days 3, 6, and 9 (i.e., for Protocols 3 and 4); or 200 mL per liter of cell culture fluid on day 3 (i.e., for Protocols 1 and 2). The batch feed medium did not contain Cys or Cys-Cys. As noted in Table 1, Cys or Cys-Cys was supplied to the production cultures in the basal media and via supplementation from a stock solution (i.e., 10 ml of 450 mM Cys or 10 mls of 225 mM Cys-Cys) on the same days as the batch feed medium was supplied. Cysteine or cystine was supplied in an amount such that the total cysteine monomer potential for all production runs was kept equivalent (i.e., 2× cysteine (Cys) concentration vs. lx cystine (Cys-Cys) for either a 1×20% batch feed strategy or a 3×10% batch feed strategy.

The concentration of glucose was analyzed every day and if the glucose concentration fell below 3 g/L, it was replenished from a 500 g/L stock solution of glucose for prevention of glucose depletion. Reactors were equipped with calibrated dissolved oxygen, pH, and temperature probes. Dissolved oxygen was controlled on-line through sparging with air and/or oxygen. pH was controlled through addition of CO₂ or Na₂CO₃ and antifoam was added to the cultures as needed. The cell cultures were maintained at pH 7.0 and a temperature of 37° C. from days 0 through 3, and then at 33° C. after day 3. The cell cultures were agitated at 275 rpm and the dissolved oxygen level was at 30% of air saturation. Samples were taken daily for offline measurements. Offline osmolality, pH, and metabolite, viable cell density (VCC), cell viability were measured daily using a BioProfile FLEX Analyzer (Nova Biomedical, Waltham, MA)packed cell volume (PCV) after centrifugation of cell suspension for 10 min at ˜700 x g were also measured. In addition, supernatant samples were taken daily from day 6 to day 14 to determine product concentration using a protein A based HPLC method. Supernatant samples were taken on days 0, 3, 4, 6, 8, 10, 12, and 14 to determine extracellular amino acid concentrations using an amino acid derivatization method followed by a RP-HPLC method.

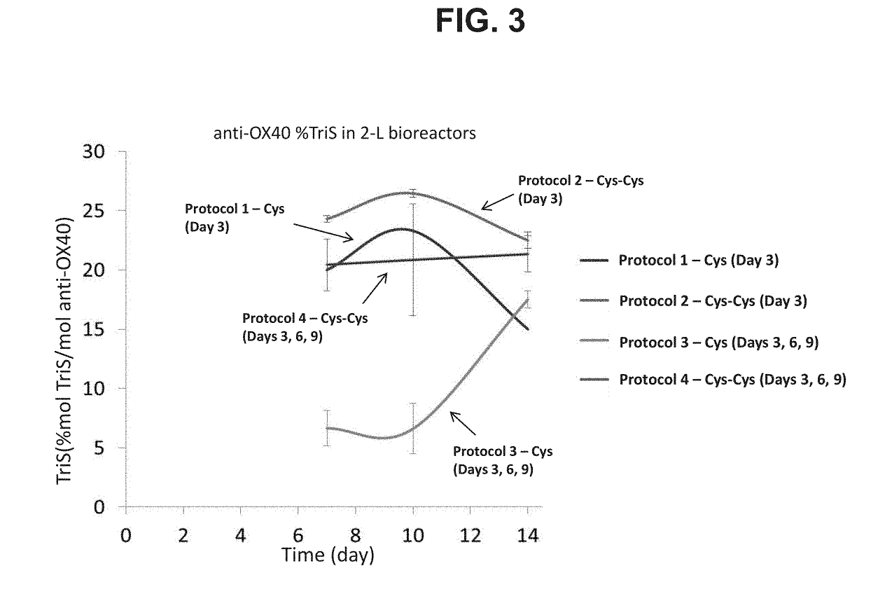

Samples were taken from each culture on Days 7, 10 and 14, and % trisulfide in anti-OX40 Ab at each time point was determined via HILIC-CAD, as described above. As shown in FIG. 3, % trisulfide was highest in anti-OX40 Ab produced by cells cultured according to Protocols 2 and 4. % trisulfide in anti-OX40 Ab produced by cells cultured according to Protocol 1 increased steadily between Day 7-Day 10 and subsequently decreased to about 15% at harvest on Day 14. % trisulfide in anti-OX40 Ab produced by cells cultured according to Protocol 3 remained low from Day 7 to Day 10 and increased to about 17.5% at harvest on Day 14. Without intending to be bound by one particular theory, the results shown in FIG. 3 suggest that in a cell culture production process using the Cys form leads to lower trisulfide bond levels when Cys is added (non-cellular mechanism). The rise in trisulfide bond levels at the end of the culture is likely due to no cysteine in the reduced form being left to reduce the trisulfides, with the result that the non-cellular mechanism drives the process toward trisulfide formation late in the run after Cys feeding. As such, the results suggest that providing cysteine early in the cell culture run can lead to lower trisulfide bond levels in the harvested polypeptide.

### B. Optimizing Cysteine/Cystine Supply to Control Trisulfides

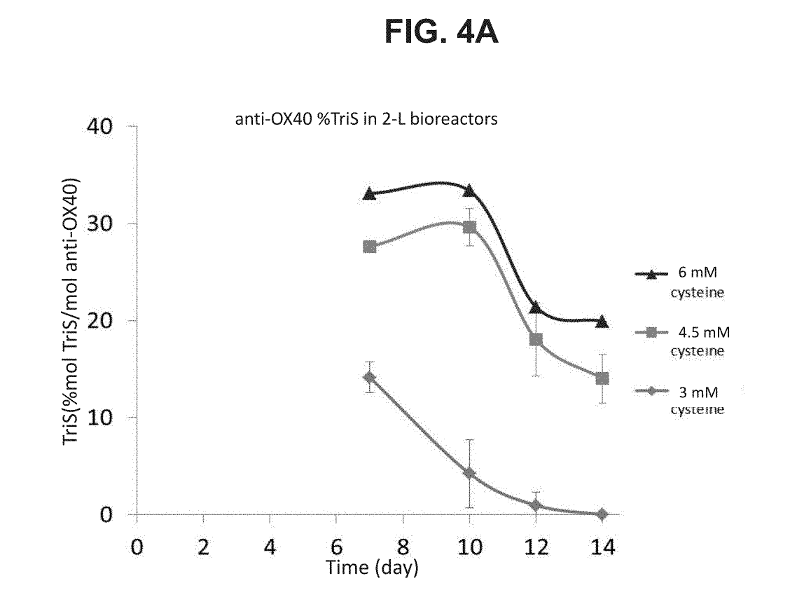

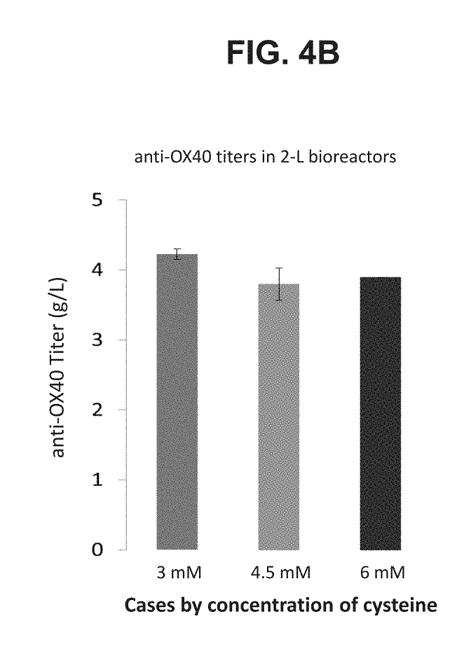

Additional experiments were performed to assess the impact of cysteine concentration when supplied only in the basal medium of the production cell culture run on trisulfide formation. CHO cells producing anti-OX40 Ab were cultured in 2 liter bioreactors over a 14 day production cell culture run. The basal medium was supplemented with (a) 6 mM cysteine, (b) 4.5 mM cysteine, or (c) 3 mM cysteine. In these experiments, the cell cultures were supplied with batch feed medium lacking cysteine. Samples were taken from each culture on Days 7, 10, 12, and 14, and % trisulfide in anti-OX40 Ab at each time point was determined via HILIC-CAD, as described above. FIG. 4A demonstrates that trisulfide bond levels in anti-OX40 Ab correlated with the initial concentrations of cysteine at the start of the production cultures. % trisulfide in anti-OX40 Ab produced by cells cultured in basal medium containing 3 mM cysteine decreased steadily from Day 7 to Day 14, with 0% trisulfide at harvest on Day 14. See FIG. 4A. Anti-OX40 Ab yields from each culture were comparable. See FIG. 4B. Taken together, the results in FIGS. 4A and 4B indicate that the cysteine concentration in a basal medium can be optimized to achieve a condition at the time of harvest under which the % trisulfide in the polypeptide is significantly reduced (or even eliminated) without affecting polypeptide yield. Without intending to be bound by one particular theory, such results suggest that cysteine, when provided at lower concentrations early in the cell culture run, is consumed by the cells, thus preventing the formation of extracellular cystine that leads to trisulfide bond formation in a secreted polypeptide.

### C. Iron and B Vitamin Levels Impact Trisulfide Bond Levels

Further experiments were conducted to assess the effects of Fe concentration and/or B vitamins (e.g., riboflavin, pyridoxine, folic acid, and cyanocobalamin) concentration on trisulfide formation in polypeptide during culture. CHO cells producing anti-OX40 Ab were cultured in 2 liter bioreactors over a 14 day production cell culture run according to one of the four protocols shown in Table 2 below:

TABLE 2

| | Cysteine, Cys-Cys, Fe, and B vitamin | | |
|---|---|---|---|
| | Concentrations | | |
| | | Addition to Production | |
| | | Culture from Batch | |
| | Basal Medium‡ | Feed | Batch Feed Strategy |
| Protocol A | 6 mM Cys | 0 mM Cys | 200 mL Batch Feed |
| (6 mM Cys, High Fe, | 0 mM Cys-Cys | 0 mM Cys-Cys | Medium added on Day 3 |
| High B vitamins) | 35 μM Fe | 0 μM Fe | |
| | B vitamins* | B vitamins** | |
| Protocol B | 6 mM Cys | 0 mM Cys | 200 mL Batch Feed |
| (6 mM Cys, Low Fe, Low | 0 mM Cys-Cys | 0 mM Cys-Cys | Medium added on Day 3 |
| B vitamins) | 20 μM Fe | 0 μM Fe | |
| | B vitamins* | 0 μM B vitamins | |
| Protocol C | 3 mM Cys | 0 mM Cys | 200 mL, Batch Feed |
| (3 mM Cys, High Fe, | 0 mM Cys-Cys | 0 mM Cys-Cys | Medium added on Day 3 |
| High B vitamins) | 35 μM Fe | 0 μM Fe | |
| | B vitamins* | B vitamins** | |

‡at the start of production culture
*B vitamins in basal medium = 1.84 μM vitamin B2, 24.9 μM vitamin B6, 22.5 μM vitamin B9, and 2.25 μM vitamin B12
**B vitamins in batch feed medium = 12.5 μM vitamin B2, 250 μM vitamin B6, 150 μM vitamin B9, and 10 μM vitamin B12

To initiate the growth phase of production cell cultures, CHO cells were inoculated at approximately 1.0×10⁶ cells/mL in 2-L stirred bioreactors (Applikon, Foster City, Calif.) containing 1L of basal medium. Dissolved oxygen, pH, temperature, agitation conditions were the same as described above. Glucose concentration, osmolality, pH, metabolite concentrations, viable cell density (VCC), cell viability, and packed cell volume after were measured as described above. In addition, supernatant samples were taken daily from day 6 to day 14 to determine product concentration using a protein A based HPLC method. Supernatant samples were taken on days 0, 3, 4, 6, 8, 10, 12, and 14 to determine extracellular amino acid concentrations using an amino acid derivatization method followed by a RP-HPLC method.

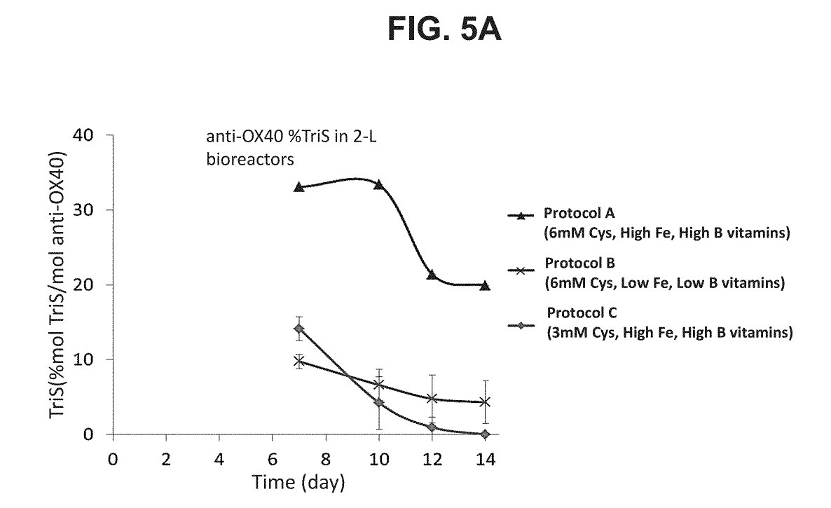

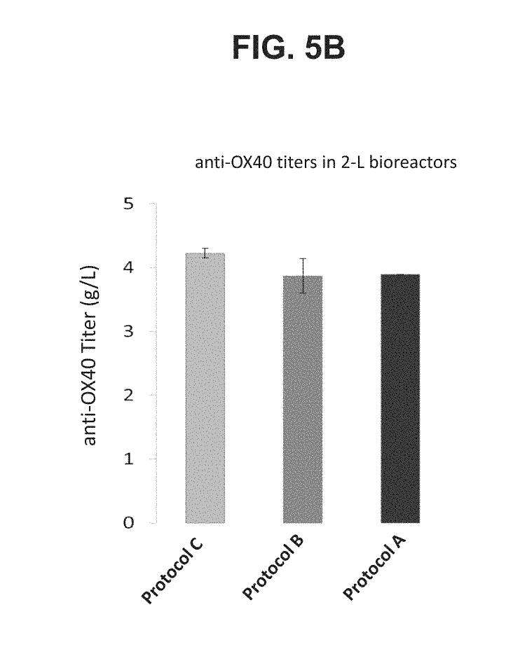

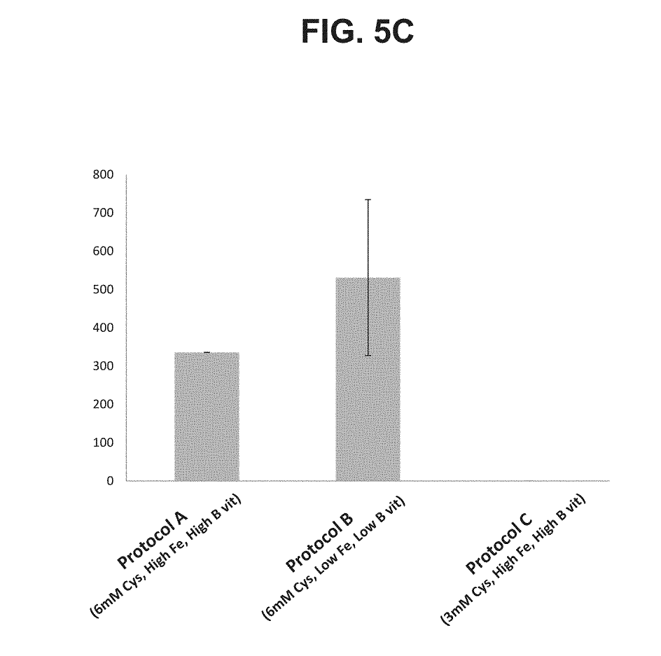

Samples were taken from each culture on Days 7, 10, 12, and 14, and % trisulfide in anti-OX40 Ab at each time point was determined via HILIC-CAD, as described above. As shown in FIG. SA, % trisulfide was highest in anti-OX40 Ab produced by cells cultured according to Protocol A. % trisulfide in anti-OX40 Ab produced by cells cultured according to Protocol C decreased steadily from Day 7 to Day 14, with 0% trisulfide at harvest on Day 14. Notably, trisulfide bond levels in anti-OX40 Ab produced by cells cultured according to Protocol B decreased from about 10% at Day 7 to about 5% on Day 14. The anti-OX40 Ab yields from each culture were comparable. See FIG. 5B.

FIG. 5C shows the residual concentrations of cystine (Cys-Cys) in the medium at the end of each cell culture. At Day 14, high levels of Cys-Cys were measured in the media of Protocols A and B, whereas no Cys-Cys was detected in the medium of Protocol C. Such results are consistent with the fact that the low concentration of Cys in basal medium used in Protocol C was entirely consumed by cells during culture, and that the high concentrations of Cys provided in the basal media in Protocols A and B were not entirely consumed by the cells during culture, thus leading to the oxidation of the remaining Cys to Cys-Cys. Notably, the high residual Cys-Cys at Day 14 in Protocol B did not lead to increased trisulfide formation in anti-OX40 Ab. Collectively, these results demonstrate that by controlling B vitamin and Fe concentrations that trisulfide bond levels can be reduced, even in the presence of high levels of Cys, a condition that over time can result in high trisulfide bond levels as Cys is converted to Cys-Cys. By controlling B vitamin and Fe concentration, trisulfide bond levels can be reduced to levels similar to those obtained by optimizing Cys concentrations in the basal medium such that there is minimal residual Cys to be converted to Cys-Cys.

### D. Relative Effect of B Vitamins and Iron in Cell Culture

To determine the relative contribution of B vitamins and Fe on trisulfide formation, CHO cells producing anti-OX40 Ab were cultured in 2 liter bioreactors containing 1 liter of basal medium over a 14 day production cell culture under the conditions described above, and run according to one of the four protocols shown in Table 3 below:

TABLE 3

| | Cysteine, Fe, and B vitamin | | |
|---|---|---|---|
| | Concentrations*** | | |
| | | Addition to Production | |
| | | Culture from the Batch | |
| | Basal Medium‡ | Feed | Batch Feed Strategy |
| Protocol D | 7.5 mM Cys | 0 mM Cys | 200 mL Batch Feed |
| (Low Fe, Low B vit) | 20 μM Fe | 0 μM Fe | Medium added on Day 3 |
| | B vitamins* | 0 μM B vitamins | |
| Protocol E | 7.5 mM Cys | 0 mM Cys | 200 mL Batch Feed |
| (Low Fe, High B vit) | 20 μM Fe | 0 μM Fe | Medium added on Day 3 |
| | B vitamins* | B vitamins** | |
| Protocol F | 7.5 mM Cys | 0 mM Cys | 200 mL Batch Feed |
| (High Fe, Low B vit) | 50 μM Fe | 0 μM Fe | Medium added on Day 3 |
| | B vitamins* | 0 μM B vitamins | |
| Protocol G | 7.5 mM Cys | 0 mM Cys | 200 mL Batch Feed |
| (High Fe, High B vit) | 50 μM Fe | 0 μM Fe | Medium added on Day 3 |
| | B vitamins* | B vitamins** | |

‡at the start of production culture
*B vitamins in basal medium = 1.84 μM vitamin B2, 24.9 μM vitamin B6, 22.5 μM vitamin B9, and 2.25 μM vitamin B12
**B vitamins in batch feed medium = 12.5 μM vitamin B2, 250 μM vitamin B6, 150 μM vitamin B9, and 10 μM vitamin B12
***No rystine (Cys-Cys) was provided in the basal medium or during batch feed.

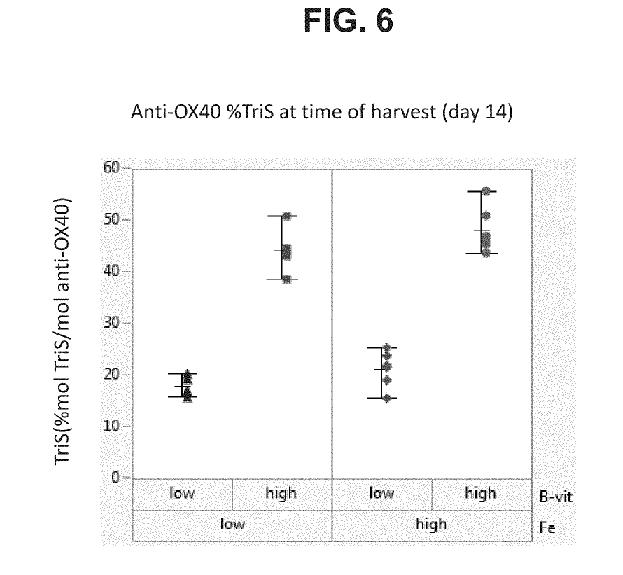

Samples of anti-OX40 Ab were taken at Days 10, 12, and at harvest on Day 14, and % trisulfide in anti-OX40 Ab for each sample was determined via HILIC-CAD, as described above. As shown in FIG. 6, % trisulfide was lowest in harvested anti-OX40 Ab produced by cells cultured according to Protocols D (i.e., low Fe, low B Vitamins) and F (i.e., high Fe and low B vitamins). Anti-OX40 Ab produced according to Protocol D had about 17-20% trisulfide, and anti-OX40 Ab produced according to Protocol F had about 17-25% trisulfide. % trisulfide in anti-OX40 Ab produced by cells cultured according to Protocol G (i.e., high Fe, high B vitamins) was about 45%-55%. By contrast to the results presented in FIGS. 2A and 2B, % trisulfide in anti-OX40 Ab produced by cells cultured according to Protocol E (i.e., low Fe, high B vitamins) was about 35%-50%. Similar results were seen in samples taken on Days 10 and 12 (data not shown). Taken together with the results shown in FIGS. 2A and 2B, which show that the B vitamins have no non-cellular effect, the results shown in FIG. 6 indicate that B vitamins make a significant contribution to the formation of trisulfide bonds and do so through a cell-related mechanism.

### Example 3

### Effect of Incubating Pre- and Post-Harvest Cell Culture Fluid of a Recombinantly Expressed Polypeptide with a Reducing Agent and a Chelating Agent on Trisulfide Formation in the Polypeptide

Further experiments were performed to identify strategies to mitigate trisulfide bond formation in harvested polypeptides. A harvested cell culture fluid (HCCF) of anti-OX40 Ab was incubated under one of the conditions outlined in Table 4 below. Each condition was controlled for temperature, pH, and dissolved oxygen (DO). Under conditions 2 and 3, the HCCF was incubated with EDTA (i.e., an exemplary metal chelating agent) for 30 minutes before cysteine (i.e., an exemplary reducing agent) was added. The mixtures were each held for 4.5h to simulate the duration of a typical cell culture harvest. Samples were transferred to a 15° C. water bath and held up to 4d (96h) to simulate chilled hold time prior to downstream purification.

TABLE 4

| | | pH | | | |
|---|---|---|---|---|---|
| | Temperature | Set | DO‡ | Reducing | Chelating |
| | Set point | point | Set point | Agent | Agent |
| Condition 1 | 33° C. | 7.0 | 30% | 6 mM Cys | — |
| (+Cys) | | | | | |
| Condition 2 | 33° C. | 7.0 | 30% | 6 mM Cys | 20 mM |
| (+Cys +EDTA) | | | | | EDTA |
| Condition 3 | 20° C. | 7.0 | 30% | 6 mM Cys | 20 mM |
| (+Cys +EDTA) | | | | | EDTA |

‡DO = dissolved oxygen; the controller acts to maintain DO levels at or above the indicated set point.

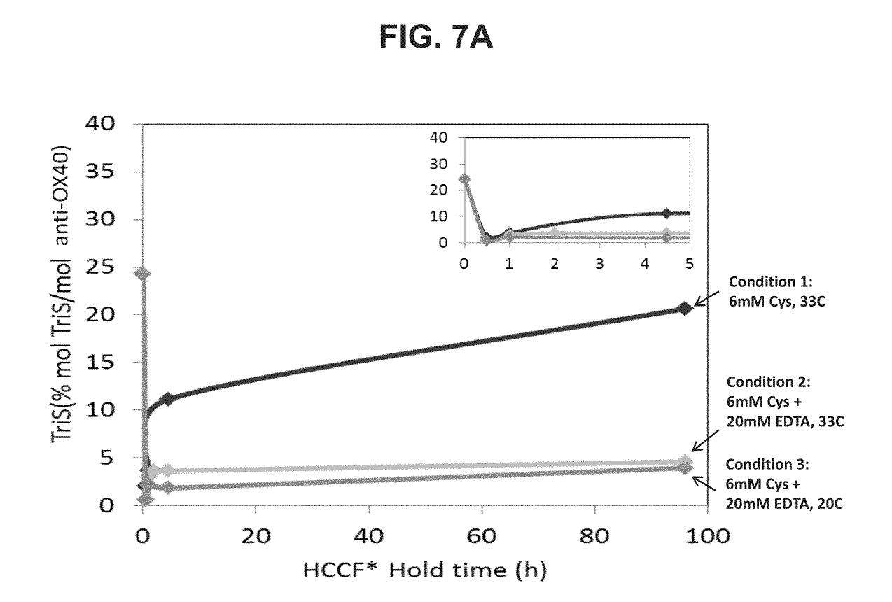

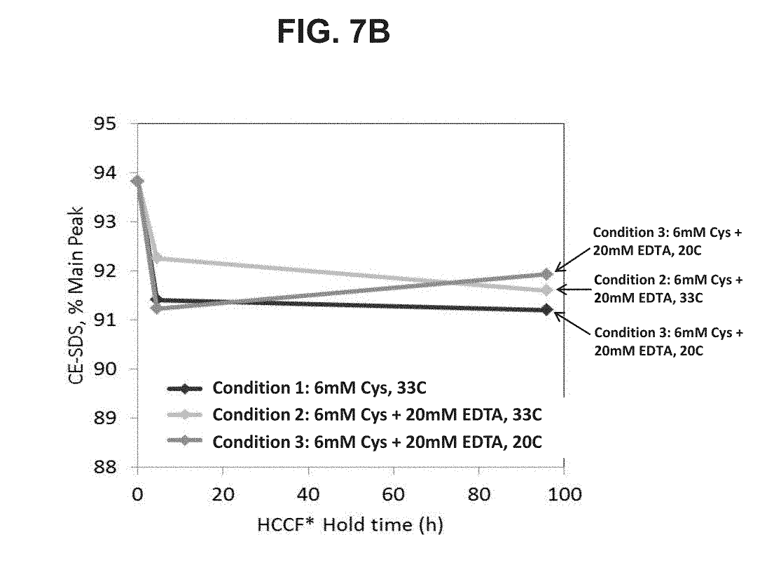

As shown in FIG. 7A, addition of cysteine (Cys) to the HCCF in Condition 1 decreased % trisulfide in anti-OX40 Ab from 24% to 2% within the first 30 minutes of the incubation, measured relative to the time of Cys addition. Trisulfide bond levels then rose to about 11% after 4.5 hours at 33° C., and increased steadily to about 21% after holding at 15° C. for 4 days. Under Condition 2, the addition of Cys to HCCF that had been incubated with EDTA decreased % trisulfide in anti-OX40 Ab from 24% to <1% within the first 30 minutes of the incubation at 33° C. Trisulfide bond levels rose to about 5% at the end of the 4 day hold. Under Condition 3, addition of Cys to the HCCF that had been incubated with EDTA also decreased % trisulfide in anti-OX40 Ab from 24% to <1% within the first 30 minutes of the incubation at 20° C. Trisulfide bond levels rose to about 2% after 4.5 hours at 20° C., and rose further to about 4% after the 4 day hold at 15° C. The addition of Cys and EDTA to the HCCF resulted in a slight decrease in main peak by CE-SDS, potentially indicating a small amount of protein reduction with the addition of reducing agent. See FIG. 7B.

Similar studies were also performed with cell culture fluid (CCF) prior to harvest. CCF was incubated with or without chelator for about 45min followed by supplementation with or without reducing agent under conditions controlled for temperature, pH, and dissolved oxygen (DO) as outlined in Table 5. This mixture was held for 4.5h, then centrifuged and filtered to remove cells. After cell removal, samples were held up to 4 days (96h) at 15° C.

TABLE 5

| | Temperature | pH Set | DO‡ | Reducing | Chelating |
|---|---|---|---|---|---|
| | Set point | point | Set point | Agent | Agent |
| Condition A | 33° C. | 7.0 | 30% | — | — |
| Condition B | 33° C. | 7.0 | 30% | 6 mM Cys | — |
| Condition C | 33° C. | 7.0 | 30% | 6 mM Cys | 20 mM |
| | | | | | EDTA |
| Condition D | 33° C. | 7.0 | 30% | 6 mM Cys | 20 mM |
| | | | | | NTA |
| Condition E | 33° C. | 7.0 | 30% | 6 mM Cys | 20 mM |
| | | | | | EDDS |
| Condition F | 33° C. | 7.0 | 30% | 6 mM Cys | 20 mM |
| | | | | | citrate |

‡DO = dissolved oxygen; the controller acts to maintain DO levels at or above the indicated set point.

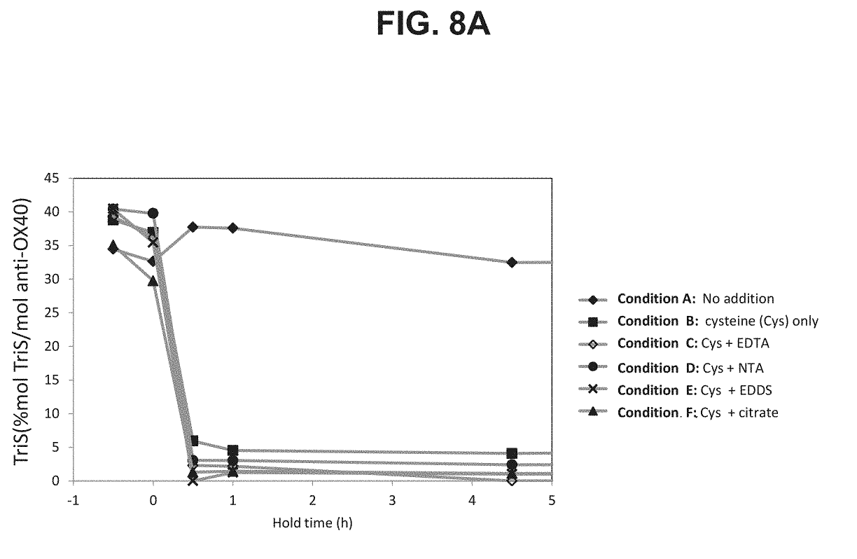

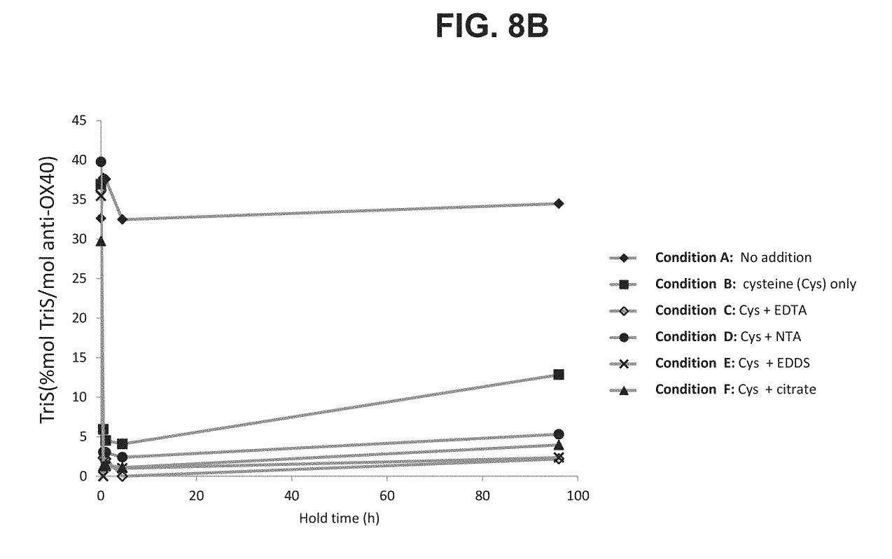

As shown in FIGS. 8A and 8B, trisulfide bond levels in the CCF that was not supplemented with a reducing agent or a chelating agent (i.e., Condition A) remained high (about 35%). Addition of Cys alone to the CCF (i.e., Condition B) decreased % trisulfide in anti-OX40 Ab from 37% to 6% within the first 30 minutes of the incubation. Trisulfide bond levels then rose to about 4% after 4.5 hours at 33° C., and increased steadily to about 13% post-harvest at the end of the 4d hold at 15° C. Addition of Cys and any one of EDTA, NTS, EDDS, or citrate to the CCF (i.e., Conditions C, D, E, and F) also decreased % trisulfide in anti-OX40 Ab from about 30-40% to 3% or lower within the first 30 minutes of the incubation. Trisulfide bond levels then stayed low, i.e., at or below 5%, through the 4.5 hour incubation at 33° C. and after the post-harvest 4 day hold at 15° C.

Taken together, the results shown in FIGS. 7 and 8 demonstrate that the addition of a reducing agent decreases % trisulfide in a polypeptide in HCCF or CCF, and that the addition of a chelating agent is required to maintain low trisulfide bond level. Moreover, the decrease in trisulfide bond levels is not accompanied by significant protein reduction.

### Example 4

### Effect of Hypotaurine on Trisulfide Formation During Polypeptide Production

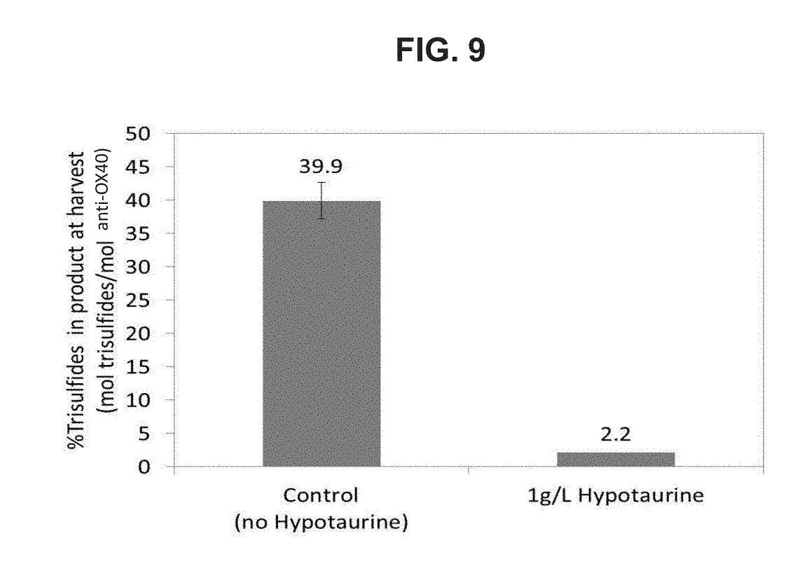

To test the effect of hypotaurine on trisulfide formation in a recombinant polypeptide during production, CHO cell cultures producing an antibody product were executed in a process known to generate polypeptides having high trisulfide bond levels (i.e., 25%-45% trisulfide). Cells were sourced from a single inoculum train and used to inoculate four replicate production cultures. Three cultures were performed under control conditions without hypotaurine. The remaining culture included 1 g/L hypotaurine in the basal medium. All other media/solution additions and process parameters were the same for all four cultures. Cell growth did not appear significantly impacted; however, viability was better maintained at the end of culture for the condition including hypotaurine (data not shown). Trisulfide bond levels in the final harvested product were significantly lower for the culture including hypotaurine: 2.2% versus 39.9 ±2.7% for the control conditions. See FIG. 9.

### Example 5

### Effect of Amino Acids that Play a Key Role in Sulfur Metabolism on Trisulfide Formation During Polypeptide Production

To assess the effects of methionine and cysteine on trisulfide formation in a recombinant polypeptide during production, CHO cells producing BsAb1 were cultured via automated robotic cell culture system in shaken 24 deep-well plates at 37° C. and 7% CO₂ (inoculum =6×10⁶ viable cells/ml) over a 14 day production cell culture run according to one of the protocols shown in Table 6 below (RTE =trace element solution, Cys quantities are first: medium and second: feed). The cultures were fed 10 mM methionine at days 3, 6, and 9 at 10% culture volume. 36 conditions were tested in total.

TABLE 6*

| | Pattern | Ser | Met | Cys | RTE |
|---|---|---|---|---|---|
| 1 | +−−+ . . . | 1 | −1 | v1.0 (3 mM, 15 mM) | 1.2 |
| 2 | ++−− . . . | 1 | 1 | v1.0 (3 mM, 15 mM) | 1.0 |
| 3 | +−++ . . . | 1 | −1 | v1.3 (7.5 mM, 0 mM) | 1.2 |
| 4 | −−−−+ . . . | −1 | −1 | v1.0 (3 mM, 15 mM) | 1.2 |
| 5 | −−−− . . . | −1 | −1 | v1.0 (3 mM, 15 mM) | 1.0 |
| 6 | −−+− . . . | −1 | −1 | v1.3 (7.5 mM, 0 mM) | 1.0 |
| 7 | −++− . . . | −1 | 1 | v1.3 (7.5 mM, 0 mM) | 1.0 |
| 8 | +−−− . . . | 1 | −1 | v1.0 (3 mM, 15 mM) | 1.0 |
| 9 | ++−− . . . | 1 | 1 | v1.0 (3 mM, 15 mM) | 1.0 |
| 10 | +−+− . . . | 1 | −1 | v1.3 (7.5 mM, 0 mM) | 1.0 |
| 11 | −+−+ . . . | −1 | 1 | v1.0 (3 mM, 15 mM) | 1.2 |
| 12 | ++++ . . . | 1 | 1 | v1.3 (7.5 mM, 0 mM) | 1.2 |
| 13 | −+−− . . . | −1 | 1 | v1.0 (3 mM, 15 mM) | 1.0 |
| 14 | −−+− . . . | −1 | −1 | v1.3 (7.5 mM, 0 mM) | 1.0 |
| 15 | −+++ . . . | −1 | 1 | v1.3 (7.5 mM, 0 mM) | 1.2 |
| 16 | −+−− . . . | −1 | 1 | v1.0 (3 mM, 15 mM) | 1.0 |
| 17 | −+−+ . . . | −1 | 1 | v1.0 (3 mM, 15 mM) | 1.2 |
| 18 | ++−− . . . | 1 | 1 | v1.0 (3 mM, 15 mM) | 1.0 |
| 19 | +++− . . . | 1 | 1 | v1.3 (7.5 mM, 0 mM) | 1.0 |
| 20 | ++−+ . . . | 1 | 1 | v1.0 (3 mM, 15 mM) | 1.2 |
| 21 | −−−+ . . . | −1 | −1 | v1.0 (3 mM, 15 mM) | 1.2 |
| 22 | +++− . . . | 1 | 1 | v1.3 (7.5 mM, 0 mM) | 1.0 |
| 23 | +−−+ . . . | 1 | −1 | v1.0 (3 mM, 15 mM) | 1.2 |
| 24 | −−++ . . . | −1 | −1 | v1.3 (7.5 mM, 0 mM) | 1.2 |
| 25 | −−++ . . . | −1 | −1 | v1.3 (7.5 mM, 0 mM) | 1.2 |
| 26 | −+++ . . . | −1 | 1 | v1.3 (7.5 mM, 0 mM) | 1.2 |
| 27 | +−−− . . . | 1 | −1 | v1.0 (3 mM, 15 mM) | 1.0 |
| 28 | ++−− . . . | 1 | 1 | v1.0 (3 mM, 15 mM) | 1.0 |
| 29 | ++++ . . . | 1 | 1 | v1.3 (7.5 mM, 0 mM) | 1.2 |
| 30 | −−−− . . . | −1 | −1 | v1.0 (3 mM, 15 mM) | 1.0 |
| 31 | +−++ . . . | 1 | −1 | v1.3 (7.5 mM, 0 mM) | 1.2 |
| 32 | ++−− . . . | 1 | 1 | v1.0 (3 mM, 15 mM) | 1.0 |
| 33 | ++−+ . . . | 1 | 1 | v1.0 (3 mM, 15 mM) | 1.2 |
| 34 | +−+− . . . | 1 | −1 | v1.3 (7.5 mM, 0 mM) | 1.0 |
| 35 | −++− . . . | −1 | 1 | v1.3 (7.5 mM, 0 mM) | 1.0 |
| 36 | ++−− . . . | 1 | 1 | v1.0 (3 mM, 15 mM) | 1.0 |

The concentration of Ser given as “−1” = 1: 7.64 mM, and the concentration of Ser given as “1” = 4.5 mM;
The concentration of Met given as “−1” = 1.58 mM, and the concentration of Met given as “1” = 2.25 mM;
Cys concentrations are given as (medium, feed), where medium concentration is 3 mM or 7.5 mM, and where feed concentration is 15 mM or 0 mM;
RTE = trace element solution

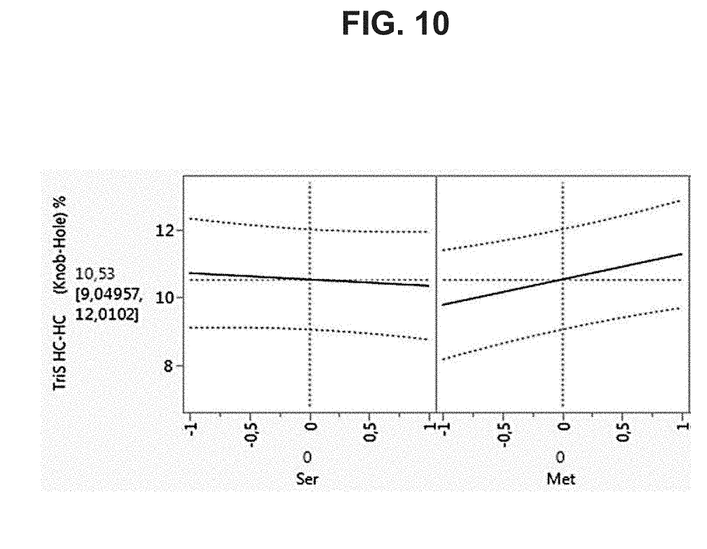

Sorted parameter estimates of the impact of methionine and serine on trisulfide bond level reduction based on robotic results were calculated with Software JMP. FIG. 10 provides a prediction profiler showing a significant impact of lowering methionine concentration on trisulfide reduction. (Calculation based on robotics data). Serine concentrations were not found to have an effect of trisulfide reduction in BsAb1.

Next, CHO cells producing BsAb1 were cultured in 2 liter bioreactors under the conditions described above according to one of the two protocols shown below in Table 7 below:

TABLE 7

| | | | | Feed |
|---|---|---|---|---|
| | Basal Medium Concentrations | | | Concentration |
| | Cysteine | Serine | Methionine | Cysteine |
| | (mM) | (mM) | (mM) | (mM) |
| Protocol H | 3 | 4.5 | 2.25 | 15 |
| Protocol I | 0 | 7.64 | 0 | 0 |

For pH control, a carbonate buffer system within the medium, CO₂ gassing, and 1 M NaHCO₃ solution were used. pO₂ was controlled using a three step aeration rate, stirrer speed, and oxygen gassing cascade.

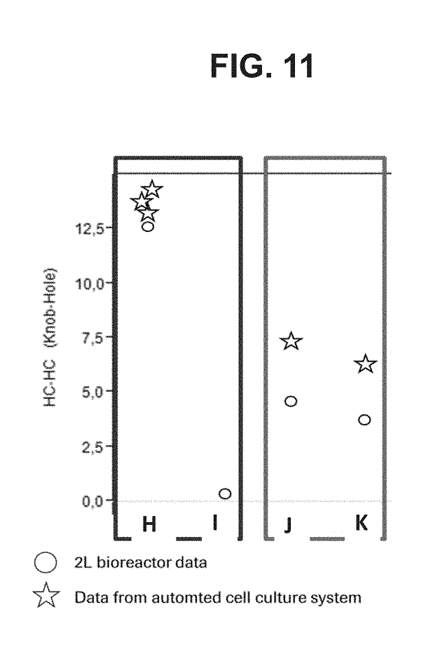

In protocol I, the sulfur-containing amino acids cysteine and methionine were omitted from the basal medium, cysteine was omitted from the feed medium, and the serine concentration was increased to compensate for the lack of cysteine. As shown in FIG. 11, omitting sulfur containing amino acids led to 96% decrease in the level of trisulfide (i.e., from 12.5% to 0.5%) in the knob-and-hold region in BsAb1 at harvest. Such results were consistent with those observed in both the automated cell culture system.

Next, CHO cells expressing BsAb1 were cultured as described above according to one of the two protocols provided in Table 8 below:

TABLE 8*

| | Basal Medium Concentrations | | |
|---|---|---|---|
| | Cysteine | Serine | Methionine |
| | (mM) | (mM) | (mM) |
| Protocol J | 7.5 | 4.5 | 2.25 |
| Protocol K | 7.5 | 7.64 | 1.58 |

*The medium in which the cells were grown in Protocols J and K was different from the medium used in Protocols H and I.

As shown in FIG. 10, decreasing the methionine concentration in the basal medium led to a 17.4% decrease in the level of trisulfide (i.e., from 4.6% to 3.8%) the knob-and-hole region in BsAb1 at harvest. Such results were consistent with those observed in the automated cell culture system.

### Example 6

### Relative Effect of B Vitamin Levels on Trisulfide Formation in a Polypeptide Produced by a Mammalian Cell

To assess the relative contribution of B vitamins on trisulfide formation on a polypeptide produced by a second mammalian cell line, CHO cells producing an antibody were cultured in 2 liter bioreactors containing 1.2 liter of basal medium over a 14 day production cell culture run according to one of the two protocols shown in Table 9 below:

TABLE 9‡

| | B Vitamin Concentration in | |
|---|---|---|
| | Batch Feed Medium* | Batch Feed Strategy |
| Protocol L | Vitamin B2: 12.5 μM | 100 mL/L Batch Feed |
| | Vitamin B6: 250 μM | Medium added on Days 3, 6, |
| | Vitamin B9: 150 μM | and 9, respectively |
| | Vitamin B12: 10 μM | |
| | (No Cys or Cys-Cys) | |
| Protocol M | Vitamin B2: 0 μM | 100 mL/L Batch Feed |
| | Vitamin B6: 0 μM | Medium added on Days 3, 6, |
| | Vitamin B9: 0 μM | and 9, respectively |
| | Vitamin B12: 0 μM | |
| | (No Cys or Cys-Cys) | |

‡B vitamins in basal medium = 1.84 μM vitamin B2, 24.9 μM vitamin B6, 22.5 μM vitamin B9, and 2.25 μM vitamin B12. The basal medium also contained 6 mM Cys.

To initiate the growth phase of production cell cultures, CHO cells were inoculated at approximately 1.0×10⁶ cells/mL in 2-L stirred bioreactors (Sartorius, Goettingen, Germany) containing 1.2L of basal medium. The cells were cultured in fed-batch mode with batch feed medium additions of either 100 mL per liter of cell culture fluid at days 3, 6, and 9. The batch feed medium did not contain Cys or Cys-Cys. 6 mM Cys was supplied to the production cultures in the basal media.

The concentration of glucose was analyzed every day and if the glucose concentration fell below 3 g/L, it was replenished from a 500 g/L stock solution of glucose for prevention of glucose depletion. Reactors were equipped with calibrated dissolved oxygen, pH, and temperature probes. Dissolved oxygen was controlled on-line through sparging with air and/or oxygen. pH was controlled through addition of CO₂ or Na₂CO₃. The cell cultures were maintained at pH 7.0 and a temperature of 37° C. The cell cultures were agitated at 233 rpm and the dissolved oxygen level was at 30% of air saturation. Samples were taken from Day 14 and % trisulfide in the antibody was determined

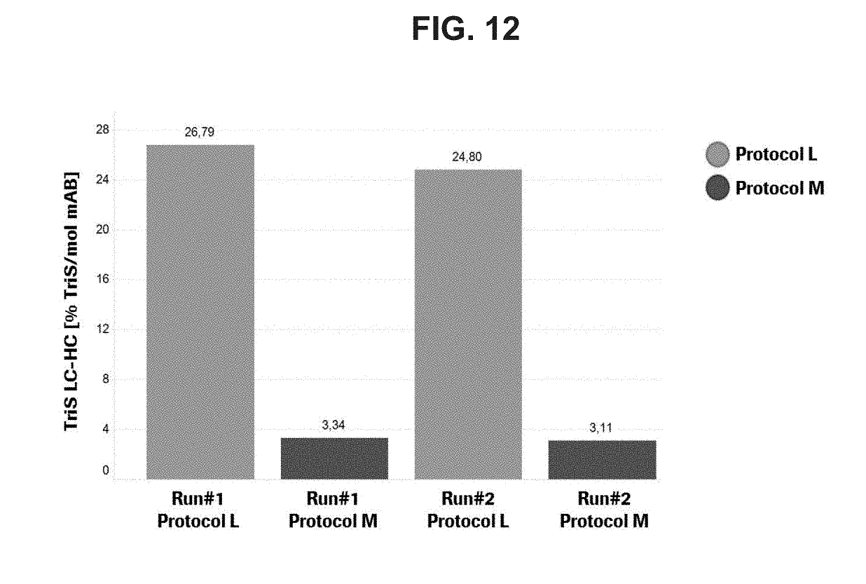

As shown in FIG. 12, decreasing the B vitamin concentration in the culture by omitting B vitamins from the batch feed medium leads to a significant reduction of trisulfide concentration by 87.5% (i.e. from 26.79% to 3.34%). The results are consistent for two runs with each setup (biological duplicates).

The preceding Examples are offered for illustrative purposes only, and are not intended to limit the scope of the present invention in any way. Various modifications of the invention in addition to those shown and described herein will become apparent to those skilled in the art from the foregoing description and fall within the scope of the appended claims.

1. A method for decreasing trisulfide bond levels in a polypeptide comprising:
(a) contacting a host cell comprising a nucleic acid encoding the polypeptide with a basal medium, wherein the basal medium comprises one or more of the following components:
i) between about 2 μM to about 35 μM iron,
ii) between about 0.11 μM to about 2μM riboflavin (vitamin B2),
iii) between about 4.5μM to about 80 μM pyridoxine or pyridoxal (vitamin B6),
iv) between about 3.4 μM to about 23 μM folic acid (vitamin B9),
v) between about 0.2 μM to about 2.5 μM cyanocobalamin (vitamin B12),
vi) between about 9 mM and about 10 mM hypotaurine; and
vii) between about 0 and about 1.58 mM methionine;
(b) culturing the host cell to produce the polypeptide; and
(c) harvesting the polypeptide produced by the host cell.

2. A method for producing a polypeptide, comprising:
(a) contacting a host cell comprising a nucleic acid encoding the polypeptide with a basal medium, wherein the basal medium comprises one or more of the following components:
i) between about 2 μM to about 35 μM iron,
ii) between about 0.11 μM to about 2μM riboflavin (vitamin B2),
iii) between about 4.5μM to about 80 μM pyridoxine or pyridoxal (vitamin B6),
iv) between about 3.4 μM to about 23 μM folic acid (vitamin B9),
v) between about 0.2 μM to about 2.5 μM cyanocobalamin (vitamin B12),
vi) between about 9 mM and about 10 mM hypotaurine; and
vii) between about 0 and about 1.58 mM methionine;
(b) culturing the host cell to produce the polypeptide; and
(c) harvesting the polypeptide produced by the host cell.

3. The method of claim 1 or claim 2, whereby the harvested polypeptide has a trisulfide bond level less than a polypeptide produced under identical conditions, except that the concentration of the one or more components differs from the concentration specified in (a).

4. The method of claim 1, wherein the basal medium lacks cystine.

5. The method of claim 1, wherein the basal medium comprises between about 1.4 mM to 3 mM cysteine or cystine.

6. The method of claim 1, wherein the basal medium comprises between about 0 mM to about 1.58 mM methionine and between about 0 mM to about 3 mM cysteine.

7. The method of claim 1, wherein the basal medium comprises about 6 mM cysteine.

8. A method for decreasing trisulfide bond levels in a polypeptide comprising:
(a) culturing a host cell comprising a nucleic acid encoding the polypeptide in a cell culture medium, wherein the cell culture medium comprises one or more of the following components:
i) between about 2 μM to about 35 μM iron,
ii) between about 0.11 μM to about 2μM riboflavin (vitamin B2),
iii) between about 4.5μM to about 80 μM pyridoxine or pyridoxal (vitamin B6),
iv) between about 3.4 μM to about 23 μM folate/folic acid (vitamin B9),
v) between about 0.2 μM to about 2.5 μM cyanocobalamin (vitamin B12),
vi) between about 9 mM and about 10 mM hypotaurine; and
vii) between about 0 and about 4.5 mM methionine;
(b) producing the polypeptide;
(c) and harvesting the polypeptide produced by the host cell.

9. The method of claim 8, wherein the concentration of one or more of the components in the cell culture medium is the cumulative concentration of one or more additions after inoculation.

10. The method of claim 8, wherein the polypeptide is selected from the group consisting of: a CEA-IL2v immunocytokine, a FAP-IL2v immunocytokine, an anti-CEA/anti-CD3 bispecific antibody, an anti-VEGF/anti-angiopoietin bispecific antibody, an anti-Ang2/anti-VEGF bispecific antibody, an anti-05 antibody, and an anti-CD40 antibody.

11. The method of claim 8, wherein the polypeptide is selected from the group consisting of: a CEA-IL2v immunocytokine, a FAP-IL2v immunocytokine, an anti-CEA/anti-CD3 bispecific antibody, an anti-VEGF/anti-angiopoietin bispecific antibody, an anti-Ang2/anti-VEGF bispecific antibody, an anti-05 antibody, and an anti-CD40 antibody.

12. The method of claim 1, wherein the method further comprises at least one feed, and wherein the feed medium lacks one or more of the following: iron, riboflavin, pyridoxine, pyridoxal, folic acid, and cyanocobalamin.

13. The method of claim 12, wherein the feed is a batch feed.

14. The method of claim 13, wherein the batch feed medium lacks cystine.

15. The method of claim 13, wherein the batch feed medium lacks cysteine.

16. The method of claim 13, wherein the batch feed medium lacks methionine.

17. The method of claim 1, wherein the iron is ferric iron (Fe³⁺) or ferrous iron (Fe²⁺).

18. The method of claim 1, wherein the method further comprises:
(I) supplementing the culture of said host cell with a chelating agent and a reducing agent prior to harvest;
(II) supplementing a pre-harvest cell culture fluid (PHCCF) of said host cell with a chelating agent and a reducing agent; or
(III) supplementing a harvested cell culture fluid (HCCF) of said host cell with a chelating agent and a reducing agent following harvest.

19. A method for decreasing level of trisulfide bonds in a polypeptide produced by a host cell comprising:
(i) supplementing a culture of said host cell with a reducing agent and a chelating agent prior to harvest;
(ii) supplementing a pre-harvest cell culture fluid (PHCCF) of said host cell with a chelating agent and a reducing agent; or
(iii) supplementing a harvested cell culture fluid (HCCF) of said host cell with a reducing agent and a chelating agent.

20. The method of claim 18, wherein the culture, the PHCCF, or the HCCF of said host cell is supplemented with the chelating agent prior to being supplemented with the reducing agent.

21. The method of claim 20, wherein the culture, the PHCCF, or the HCCF of said host cell is supplemented with the chelating agent between about 60 minutes to about 30 minutes prior to being supplemented with the reducing agent.

22. The method of claim 18, wherein the chelating agent and the reducing agent are maintained in the culture, the PHCCF, or the HCCF of said host cell for about 30 minutes to about 4 days.

23. The method of claim 18, wherein the culture, the PHCCF, or the HCCF of said host cell is maintained at a temperature between about 15° C. and about 37° C.

24. The method of claim 18, wherein the culture, the PHCCF, or the HCCF of said host cell is maintained pH between about 6.5 to about 7.5.

25. The method of claim 18, wherein the amount of dissolved oxygen (DO) in the culture, the PHCCF, or the HCCF of said host cell is at least about 15%.

26. The method of claim 18, wherein the culture, the PHCCF, or the HCCF of said host cell is maintained at a temperature between about 15° C. and about 37° C. and at a pH between about 6.5 to about 7.5, and wherein the amount of dissolved oxygen (DO) in the culture or HCCF of said host cell is at least about 15%.

27. The method of claim 18, wherein the reducing agent is selected from the group consisting of: glutathione (GSH), L-glutathione (L-GSH), cysteine, L-cysteine, tris(2-carboxyethyl)phosphine hydrochloride (TCEP), 2,3-tert-butyl-4-hydroxyanisole, 2,6-di-tert-butyl-4-methylphenol, 3-aminopropane-l-sulfonic acid, adenosylhomocysteine, anserine, B-alanine, B-carotene, butylated hydroxyanisole, butylated hydroxytoluene, carnosine, carvedilol, curcumin, cysteamine, cysteamine hydrochloride, dexamethasone, diallyldisulfide, DL-lanthionine, DL-thiorphan, ethoxyquin, gallic acid, gentisic acid sodium salt hydrate, glutathione disulfide, glutathione reduced ethyl ester, glycine, hydrocortisone, hypotaurine, isethionic acid ammonium salt, L-cysteine-glutathione Disulfide, L-cysteinesulfinic acid monohydrate, lipoic acid, reduced lipoic acid, mercaptopropionyl glycine, methionine, methylenebis(3-thiopropionic acid), oxalic acid, quercitrin hydrate, resveratrol, retinoic acid, S-carboxymethyl-L-cysteine, selenium, selenomethionine, silver diethyldithiocarbamate, taurine, thiolactic acid, tricine, vitamin C, vitamin E, vitamin B1, vitamin B2, vitamin B3, vitamin B4, vitamin B5, vitamin B6, and vitamin B11.

28. The method of claim 27, wherein the reducing agent is selected from the group consisting of: cysteine and L-cysteine.

29. The method of claim 28, wherein the reducing agent is L-cysteine, and wherein the L-cysteine is added to the culture or HCCF of said host cell to achieve a final concentration between about 3 mM and about 6 mM.

30. The method of claim 18, wherein the chelating agent is selected from the group consisting of: ethylenediaminetetraacetic acid (EDTA), nitrilotriacetic acid (NTA), ethylenediamine-N,N′-disuccinic acid (EDDS), citrate, oxalate, tartrate, ethylene-bis(oxyethylenenitrilo)tetraacetic acid (EGTA), diethylenetriaminepentaacetic acid (DTPA), 5-sulfosalicylic acid, N,N-dimethyldodecylamine N-oxide, dithiooxamide, ethylenediamine, salicylaldoxime, N-(2′-hydroxyethyl)iminodiacetic acid (HIMDA), oxine quinolinol, and sulphoxine.

31. The method of claim 30, wherein the chelating agent is selected from the group consisting of: ethylenediaminetetraacetic acid (EDTA), nitrilotriacetic acid (NTA), ethylenediamine-N,N′-disuccinic acid (EDDS), and citrate.

32. The method of claim 31, wherein the chelating agent is added to the culture or the HCCF of said host cell to achieve a final concentration of 20 mM.

33. The method of claim 1, wherein the polypeptide is secreted into the cell culture medium.

34. The method of claim 1, further comprising a step of purifying the harvested polypeptide.

35. The method of claim 1, wherein the host cell is a recombinant host cell.

36. The method of claim 1, wherein the host cell is a mammalian cell.

37. The method of claim 36, wherein the mammalian cell is a CHO cell.

38. The method of claim 1, wherein the method further comprises measuring the level of trisulfide bonds in the polypeptide.

39. The method of claim 1, wherein the average % trisulfide bonds in the polypeptide is less than about 20%, less than about 10% less than about 5%, less than about 1%, less than about 0.5%, or less than about 0.1%.

40. The method of claim 1, wherein the polypeptide is an antibody or fragment thereof.

41. The method of claim 40, wherein the polypeptide is an antibody fragment, and wherein the antibody fragment is selected from the group consisting of: a Fab, a Fab′, an F(ab′)₂, an scFv, an (scFv)₂, a dAb, a complementarity determining region (CDR) fragment, a linear antibody, a single-chain antibody molecule, a minibody , a diabody, and multispecific antibody formed from antibody fragments.

42. The method of claim 40, wherein the antibody or fragment thereof binds to an antigen selected from the group consisting of: BMPR1B, E16, STEAP1, 0772P, MPF, Napi3b, Sema 5b, PSCA hlg, ETBR, MSG783, STEAP2, TrpM4, CRIPTO, CD21, CD79b, FcRH2, HER2, NCA, MDP, IL20Rα, Brevican, EphB2R, ASLG659, PSCA, GEDA, BAFF-R, CD22, CD79a, CXCR5, HLA-DOB, P2X5, CD72, LY64, FcRH1, IRTA2, TENB2, PMEL17, TMEFF1, GDNF-Ra1, Ly6E, TMEM46, Ly6G6D, LGR5, RET, LY6K, GPR19, GPR54, ASPHD1, Tyrosinase, TMEM118, GPR172A, CD33, CLL-1, C5, OX40, α4β7 and αEβ7 integrin heterodimers, IL-13, CD-20, FGFR, influenza A, influenza B, amyloid beta, HER3, complement factor D, IL-22c, PD-L1, PD-L2, PD-1, VEGF, Angiopoietin 2, CD3, FAP, CEA, and IL-6.

43. The method of claim 40, wherein the polypeptide is an antibody, and wherein the antibody is a bispecific antibody.

44. The method of claim 43, wherein the bispecific antibody is an anti-VEGF/anti-angiopoietin bispecific antibody, an anti-CEA/anti-CD3 bispecific antibody, or an anti-Ang2/anti-VEGF bispecific antibody.

45. The method of claim 1, wherein the polypeptide is an immunocytokine.

46. The method of claim 45, wherein the immunocytokine is CEA-IL2v or FAP-IL2v.

47. Use of between about 0 and about 4.5 μM methionine in a cell culture medium for decreasing trisulfide bond levels in a polypeptide selected from the group consisting of: a CEA-IL2v immuocytokine, a FAP-IL2v immunocytokine, an anti-CEA/anti-CD3 bispecific antibody, an anti-VEGF/anti-angiopoietin bispecific antibody, an anti-Ang2/anti-VEGF bispecific antibody, an anti-C5 antibody, and an anti-CD40 antibody.

48. A polypeptide produced according to claim 1.

49. The polypeptide of claim 48, wherein the average % trisulfide in the polypeptide is less than about 20%, less than about 10% less than about 5%, less than about 1%, less than about 0.5%, or less than about 0.1%.
